(19) United States
(12) Patent Application Publication
(10) Pub. No.: US 2023/0383324 A1
(43) Pub. Date: Nov. 30, 2023

(54) Methods for purifying bacterial polysaccharides

(71) Applicant: Pfizer Inc., New York, NY (US)

(72) Inventors: Elizabeth BARANYI, Newburgh, NY (US); Wei CHEN, Cliffside Park, NJ (US); Zecheng CHEN, New City, NY (US); Justin Keith MORAN, West Nyack, NY (US); Yonghui YUAN, Blauvelt, NY (US)

(21) Appl. No.: 18/249,411

(22) PCT Filed: Oct. 19, 2021
(86) PCT No.: PCT/IB2021/059619

§ 371 Date: Apr. 18, 2023

Related U.S. Application Data
(60) Provisional application No. 63/104,180, filed on Oct. 22, 2020.

(51) Int. Cl. C12P 19/04

(52) U.S. Cl. CPC C12P 19/04; C12R 2001/46

(57) ABSTRACT

The present invention relates to methods for purifying bacterial polysaccharides, in particular for removing impurities from cellular lysates of bacteria producing polysaccharides.

### FIELD OF THE INVENTION

The present invention relates to methods for purifying S. pneumoniae serotype 3 polysaccharide, in particular for removing impurities from cellular lysates.

### BACKGROUND OF THE INVENTION

Bacterial polysaccharides, in particular capsular polysaccharides, are important immunogens found on the surface of bacteria involved in various bacterial diseases. This has led to them being an important component in the design of vaccines. They have proved useful in eliciting immune responses especially when linked to carrier proteins.

In the preparation of multivalent conjugate pneumococcal vaccines directed to the prevention of invasive diseases caused by the organism Streptococcus pneumoniae (also known as pneumococcus), selected Streptococcus pneumoniae serotypes are grown to supply polysaccharides needed to produce the vaccine. The capsular polysaccharide is then purified. After conjugation with a carrier protein, the polysaccharide is included in the final vaccine product and confers immunity in the vaccine's target population to the selected Streptococcus pneumoniae serotypes.

The polysaccharide needs to be purified as the starting material also contains large quantities of cellular debris including DNA, RNA, proteins, and residual media components. The high burden of contaminant has been particularly problematic within runs for certain serotypes. Some serotypes, in particular Streptococcus pneumoniae serotype 3, produce large and viscous polysaccharide chains (e.g., for Type 3, chains of glucose/glucuronic acid of 2-3 million Daltons). Its viscosity has made it difficult to handle, and the purification process has been insufficient or laborious and may have resulted in loss of polysaccharide, thereby reducing yield.

Thus, there is a need for a simplified purification process of Streptococcus pneumoniae Type 3 polysaccharides to eliminate inefficiencies of the current purification processes and to produce substantially purified bacterial saccharides suitable for incorporation into vaccines.

The present invention provides a Pneumococcus Type 3 polysaccharide purification process which is simple, scalable and cost effective.

### SUMMARY OF THE INVENTION

The present invention provides a method for purifying Streptococcus pneumoniae serotype 3 polysaccharide from a solution comprising said polysaccharide together with contaminants, wherein said method comprises a base treatment step.

The starting material maybe a bacterial culture of Streptococcus pneumoniae serotype 3, in particular Streptococcus pneumoniae serotype 3 cells in suspension in their original culture medium or a wet cell paste. The solution may then be treated with a lytic agent, in particular a detergent such that the polysaccharide is released. The solution can then be treated by a base to achieve a pH above 8.0, preferably a pH above 10.0. The base may be NaOH, KOH, LiOH, NaHCO₃, Na2C03, KzC03, KCN, Et3N, NH3, HzN2H2, NaH, NaOMe, NaOEt or KOtBu. In an embodiment the base treatment step is performed at a temperature between about 4° C. and about 30° C.

Following base treatment, the suspension can be clarified by decantation, sedimentation, filtration or centrifugation and the Streptococcus pneumoniae serotype 3 polysaccharide containing solution may be further clarified by filtration or depth filtration.

The Streptococcus pneumoniae serotype 3 polysaccharide containing solution can then be further clarified by Ultrafiltration and/or Diafiltration and/or further treated by a flocculation step.

After flocculation, the suspension can be clarified by decantation, sedimentation, filtration or centrifugation.

Following the flocculation step and/or the decantation, sedimentation, filtration or centrifugation step, the pH of the polysaccharide containing solution may be adjusted to a pH above 5.0, preferably to a pH between 5.0 and 9.0 and the solution may be further clarified by an activated carbon filtration step.

The Streptococcus pneumoniae serotype 3 polysaccharide containing solution may be further clarified by Ultrafiltration and/or Diafiltration to obtain a purified Streptococcus pneumoniae serotype 3 polysaccharide solution

The purified solution of polysaccharide may be sized to a target molecular weight and/or the purified solution of polysaccharide may be sterilely filtered.

### DETAILED DESCRIPTION OF THE DRAWINGS

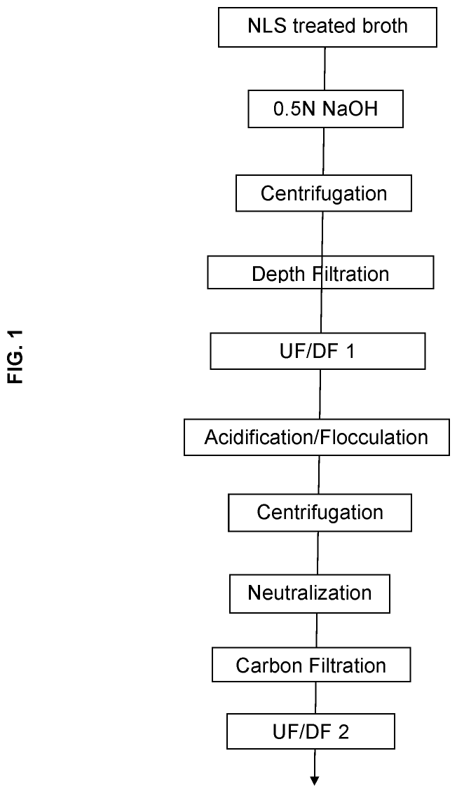

FIG. 1 Process Flow Diagram for Purification of polysaccharide

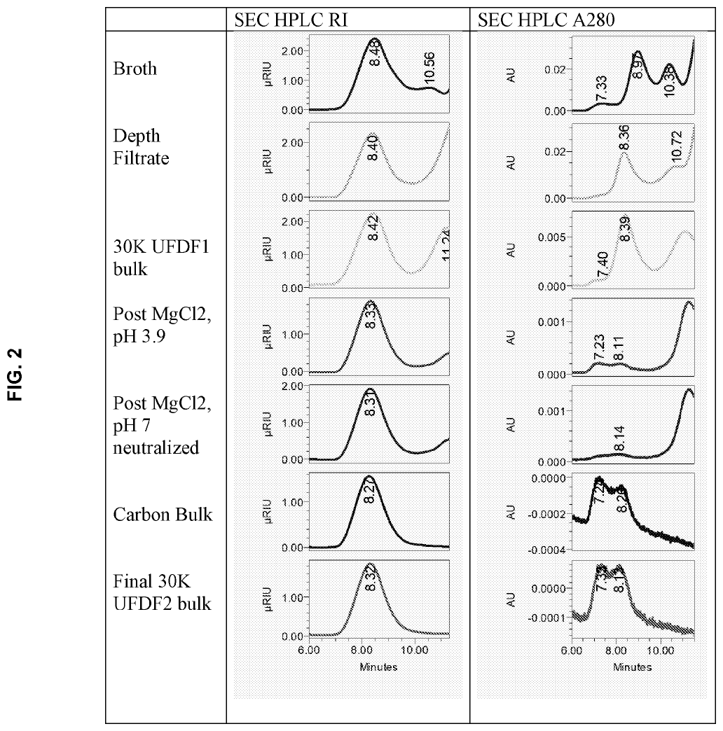

FIG. 2 Comparison of the SEC-HPLC Chromatograms after Each Purification Step of a process of the invention Monitored by Refractive Index (RI) and UV 280 nm

### DETAILED DESCRIPTION OF THE INVENTION

When applying processes of the prior art to purify Streptococcus pneumoniae serotype 3 polysaccharides from lysed broth the inventors noted difficulties during clarification. Very fine particles were observed when using processes comprising a step of adjustment to acidic pH (see e.g. WO2008118752A2) and these particles couldn't be removed from the polysaccharide solution by either centrifugation, depth filtration, or even 0.22 μm filtration. These fine particles were carried over through the next steps which resulted in a final hazy drug substance intermediate solution with higher than expected level of protein and nucleic acid impurities.

Other processes known in the art may be very long tedious processes.

The current invention provides for:
(I) Much shorter, efficient, scalable and cost-effective purification processes.
(II) Higher polysaccharide yield. Polysaccharide yield was higher than prior art processes, which lowers the overall production cost.
(III) Higher product quality. The protein/polysaccharide ratio can be about 0.3% and has similar nucleic acid and C-poly levels compared with that from the prior art processes.

The present invention applies base treatment (e.g. sodium hydroxide) to treat the fermentation broth to disrupt the association between protein and polysaccharide, and therefore allows protein to be removed in the subsequent downstream purification. This provides three distinct advantages: (i) it improves the separation efficiency, hence, clearer centrate solution for downstream clarification (e.g. via depth filtration); (ii) lipid like impurity which may be attached to the polysaccharide is removed with this step; and (iii) it removes a substantial amount of protein and nucleic acid impurities because base treatment (e.g. sodium hydroxide) can degrade endotoxin, protein and nucleic acids and these impurities are reduced.

In summary, the present invention provides a purification process which is simple, scalable and cost effective. The purified polysaccharides have very low impurity levels, and structures conform to the known reference standard by NMR spectra.

1. Purification Process of Streptococcus pneumoniae Serotype 3 Polysaccharides

1.1 Starting Material

The methods of the invention can be used to purify Streptococcus pneumoniae serotype 3 polysaccharide from a solution comprising said polysaccharide together with contaminants. In an embodiment, the contaminants are cell debris. In an embodiment, the contaminants are proteins and nucleic acids. In an embodiment, the contaminants are proteins, C-polysaccharide and nucleic acids.

1.1.1 Bacterial Cells

The source of bacterial polysaccharide to be purified according to this invention is Streptococcus pneumoniae serotype 3 bacterial cells.

A polysaccharide desired for purification may be associated with a cellular component, such as a cell wall. Association with the cell wall means that the polysaccharide is a component of the cell wall itself, and/or is attached to the cell wall, either directly or indirectly via intermediary molecules, or is a transient coating of the cell wall (for example, certain bacterial strains exude capsular polysaccharides, also known in the art as ‘exopolysaccharides’).

Bacterial strains used to purify Streptococcus pneumoniae serotype 3 polysaccharides that are used in the present invention may be obtained from established culture collections or clinical specimens.

1.1.2 Streptococcus pneumoniae Serotype 3 Bacterial Cells Growth

Typically, the Streptococcus pneumoniae serotype 3 polysaccharides is produced by growing the bacteria in a medium (e.g. a solid or preferably a liquid medium). The polysaccharide is then prepared by treating the bacterial cells.

Therefore, in an embodiment, the starting material for methods of the present invention is a bacterial culture and preferably a liquid bacterial culture (e.g. a fermentation broth). In an embodiment, the starting material for methods of the present invention is a liquid bacterial culture.

The bacterial culture is typically obtained by batch culture, fed batch culture or continuous culture (see e.g. WO 2007/052168 or WO 2009/081276). During continuous culture, fresh medium is added to a culture at a fixed rate and cells and medium are removed at a rate that maintains a constant culture volume.

The population of the organism is often scaled up from a seed vial to seed bottles and passaged through one or more seed fermentors of increasing volume until production scale fermentation volumes are reached.

1.1.3 Pre-Treatment of the Streptococcus pneumoniae Serotype 3 Bacterial Cells in Order to Obtain the Starting Material

Generally, a small amount of polysaccharide is released into the culture medium during bacterial growth, and so the starting material may thus be the supernatant from a centrifuged Streptococcus pneumoniae serotype 3 bacterial culture.

Typically, however, the starting material will be prepared by treating the bacteria themselves, such that the polysaccharide is released.

Optionally, after cell growth, the bacterial cells are deactivated. A suitable method for deactivation is for example treatment with phenol:ethanol, e.g. as described in Fattom et al. (1990) Infect Immun. 58(7):2367-74. In the below embodiments, the bacterial cells may be previously deactivated or not deactivated.

Polysaccharides can be released from bacteria by various methods, including chemical, physical or enzymatic treatment (see e.g.; WO2010151544, WO 2011/051917 or WO2007084856).

In an embodiment, the bacterial cells (deactivated or not deactivated) are treated in suspension in their original culture medium. The process may therefore start with the cells in suspension in their original culture medium.

In another embodiment the bacterial cells are centrifuged prior to release of capsular polysaccharide. The process may therefore start with the cells in the form of a wet cell paste. Alternatively, the cells are treated in a dried form. Typically, however, after centrifugation the bacterial cells are resuspended in an aqueous medium that is suitable for the next step in the process, e.g. in a buffer or in distilled water. The cells may be washed with this medium prior to re-suspension.

In an embodiment, the bacterial cells (e.g. in suspension in their original culture medium, in the form of a wet cell paste, in a dried form or resuspended in an aqueous medium after centrifugation) are treated with a lytic agent. In an embodiment, the bacterial cells in suspension in their original culture medium are treated with a lytic agent. In an embodiment, the bacterial cells resuspended in an aqueous medium after centrifugation are treated with a lytic agent. A “lytic agent” is any agent that aids in cell wall breakdown.

In an embodiment, the lytic agent is a detergent. As used herein, the term “detergent” refers to any anionic or cationic detergent capable of inducing lysis of bacterial cells. Representative examples of such detergents for use within the methods of the present invention include deoxycholate sodium (DOC), N-lauryl sarcosine (NLS), chenodeoxycholic acid sodium, and saponins (see WO 2008/118752 pages 13 lines 14 to page 14 line 10). In one embodiment of the present invention, the lytic agent used for lysing bacterial cells is DOC. In one embodiment of the present invention, the lytic agent used for lysing bacterial cells is NLS.

In an embodiment, the lytic agent is a non-animal derived lytic agent. In one embodiment, the non-animal derived lytic agent is selected from the group consisting of decanesulfonic acid, tert-octylphenoxy 5 poly(oxyethylene)ethanols (e.g. Igepal® CA-630, CAS #: 9002-93-1, available from Sigma Aldrich, St. Louis, MO), octylphenol ethylene oxide condensates (e.g. Triton® X-100, available from Sigma Aldrich, St. Louis, MO), N-lauryl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate, and cholate. In one embodiment, the non-animal derived lytic agent is decanesulfonic acid, tert-octylphenoxy 5 poly(oxyethylene)ethanols (e.g. Igepal® CA-630, CAS #: 9002-93-1, available from Sigma Aldrich, St. Louis, MO), octylphenol ethylene oxide condensates (e.g. Triton® X-100, available from Sigma Aldrich, St. Louis, MO), N-lauryl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate or cholate. In an embodiment, the non-animal derived lytic agent is NLS.

In an embodiment, the bacterial cells (e.g. in suspension in their original culture medium, in the form of a wet cell paste, in a dried form or resuspended in an aqueous medium after centrifugation) are enzymatically treated such that the polysaccharide is released. In an embodiment, the bacterial cells are treated by an enzyme selected from the group consisting of lysostaphin, mutanolysin β-N-acetylglucosaminidase and a combination of mutanolysin and β-N-acetylglucosaminidase. In an embodiment, the bacterial cells are treated by lysostaphin, mutanolysin β-N-acetylglucosaminidase or a combination of mutanolysin and β-N-acetylglucosaminidase. These act on the bacterial peptidoglycan to release the capsular saccharide for use with the invention but also lead to release of the group-specific carbohydrate antigen. In an embodiment, the bacterial cells are treated by a type II phosphodiesterase (PDE2). Optionally, after polysaccharide release, the enzyme(s) is/are deactivated. A suitable method for deactivation is for example heat treatment or acidic treatment.

In an embodiment, the bacterial cells (e.g. in suspension in their original culture medium, in the form of a wet cell paste, in a dried form or resuspended in an aqueous medium after centrifugation) are autoclaved such that the polysaccharide is released.

In a further embodiment, the bacterial cells (e.g. in suspension in their original culture medium or resuspended in an aqueous medium after centrifugation) are chemically treated such that the polysaccharide is released. In such an embodiment, the chemical treatment can be for example hydrolysis using base or acid (see e.g. WO2007084856).

In an embodiment, the bacterial cells chemical treatment is base extraction (e.g., using sodium hydroxide). Base extraction can cleave the phosphodiester linkage between the capsular saccharide and the peptidoglycan backbone. In an embodiment, the base is selected from the group consisting of NaOH, KOH, LiOH, NaHCO₃, Na2C03, KzC03, KCN, Et3N, NH3, HzN2H2, NaH, NaOMe, NaOEt and KOtBu. In an embodiment, the base comprises at least one of NaOH, KOH, LiOH, NaHC03, Na2C03, KzC03, KCN, Et3N, NH3, HzN2H2, NaH, NaOMe, NaOEt or KOtBu. After base treatment, the reaction mixture may be neutralised. This may be achieved by the addition of an acid. In an embodiment, after base treatment, the reaction mixture is neutralised by an acid selected from the group consisting of HCl, H₃PO₄, citric acid, acetic acid, nitrous acid, and sulfuric acid. In an embodiment, after base treatment, the reaction mixture is neutralised by HCl, H₃PO₄, citric acid, acetic acid, nitrous acid or sulfuric acid.

In an embodiment, the bacterial cells chemical treatment is acid treatment (e.g., sulfuric acid). In an embodiment, the acid is selected from the group consisting of HCl, H₃PO₄, citric acid, acetic acid, nitrous acid, and sulfuric acid. In an embodiment, the acid comprises at least one of HCl, H₃PO₄, citric acid, acetic acid, nitrous acid or sulfuric acid. Following acid treatment, the reaction mixture may be neutralised. This may be achieved by the addition of a base. In an embodiment, after acid treatment, the reaction mixture is neutralised by a base selected from the group consisting of NaOH, KOH, LiOH, NaHCO₃, Na2C03, KzC03, KCN, Et3N, NH3, HzN2H2, NaH, NaOMe, NaOEt and KOtBu. In an embodiment, after acid treatment, the reaction mixture is neutralised by NaOH, KOH, LiOH, NaHCO₃, Na2C03, KzC03, KCN, Et3N, NH3, HzN2H2, NaH, NaOMe, NaOEt KOtBu.

1.2 Base Treatment

The methods of the invention comprise a base treatment step. The inventors have surprisingly found that the process results in a purified polysaccharide with lower contamination. The inventor's process is simple and efficient.

In an embodiment of the present invention, the solution obtained by any of the method of section 1.1 is treated by a base to achieve a pH above 8.0. In an embodiment of the present invention, the solution obtained by any of the method of section 1.1 is treated by a base to achieve a pH above 8.5. In an embodiment of the present invention, the solution obtained by any of the method of section 1.1 is treated by a base to achieve a pH above 9.0. In an embodiment of the present invention, the solution obtained by any of the method of section 1.1 is treated by a base to achieve a pH above 9.5. In an embodiment of the present invention, the solution obtained by any of the method of section 1.1 is treated by a base to achieve a pH above 10.0. In an embodiment of the present invention, the solution obtained by any of the method of section 1.1 is treated by a base to achieve a pH above 10.5. In an embodiment of the present invention, the solution obtained by any of the method of section 1.1 is treated by a base to achieve a pH above 11.0. In an embodiment of the present invention, the solution obtained by any of the method of section 1.1 is treated by a base to achieve a pH above 11.5. In an embodiment of the present invention, the solution obtained by any of the method of section 1.1 is treated by a base to achieve a pH above 12.0. In an embodiment of the present invention, the solution obtained by any of the method of section 1.1 is treated by a base to achieve a pH above 12.5. In an embodiment of the present invention, the solution obtained by any of the method of section 1.1 is treated by a base to achieve a pH above 13.0. In an embodiment of the present invention, the solution obtained by any of the method of section 1.1 is treated by a base to achieve a pH above 13.5. In an embodiment of the present invention, the solution obtained by any of the method of section 1.1 is treated by a base to achieve a pH above 14.0. In a particular embodiment of the present invention, the solution obtained by any of the method of section 1.1 is treated by a base to achieve a pH between 8.0 and 14.0. In a particular embodiment of the present invention, the solution obtained by any of the method of section 1.1 is treated by a base to achieve a pH between 9.0 and 14.0. In a particular embodiment of the present invention, the solution obtained by any of the method of section 1.1 is treated by a base to achieve a pH between 10.0 and 14.0. In a particular embodiment of the present invention, the solution obtained by any of the method of section 1.1 is treated by a base to achieve a pH between 11.0 and 14.0. In a particular embodiment of the present invention, the solution obtained by any of the method of section 1.1 is treated by a base to achieve a pH between 12.0 and 14.0. In a particular embodiment of the present invention, the solution obtained by any of the method of section 1.1 is treated by a base to achieve a pH between 13.0 and 14.0. In a particular embodiment of the present invention, the solution obtained by any of the method of section 1.1 is treated by a base to achieve a pH between 13.0 and 13.5. In a particular embodiment of the present invention, the solution obtained by any of the method of section 1.1 is treated by a base to achieve a pH between 12.0 and 13.5. In a particular embodiment of the present invention, the solution obtained by any of the method of section 1.1 is treated by a base to achieve a pH between 11.0 and 13.5. In a particular embodiment of the present invention, the solution obtained by any of the method of section 1.1 is treated by a base to achieve a pH between 10.0 and 13.5. In a particular embodiment of the present invention, the solution obtained by any of the method of section 1.1 is treated by a base to achieve a pH between 9.0 and 13.5.

In an embodiment of the present invention, the solution obtained by any of the method of section 1.1 is treated by a base to achieve a pH of about 8.0, about 8.5, about 9.0, about 9.5, about 10.0, about 10.5, about 11.0, about 11.5, about 12.0, about 12.5, about 13.0, about 13.5 or about 14.0.

In an embodiment of the present invention, the solution obtained by any of the method of section 1.1 is treated by a base to achieve a pH of about 13.0. In an embodiment of the present invention, the solution obtained by any of the method of section 1.1 is treated by a base to achieve a pH of about 13.5. In an embodiment of the present invention, the solution obtained by any of the method of section 1.1 is treated by a base to achieve a pH of about 12.5. In an embodiment of the present invention, the solution obtained by any of the method of section 1.1 is treated by a base to achieve a pH of about 12.0.

Any number within any of the above ranges is contemplated as an embodiment of the disclosure.

In an embodiment, the base is selected from the group consisting of NaOH, KOH, LiOH, NaHC03, Na2C03, KzC03, KCN, Et3N, NH3, HzN2H2, NaH, NaOMe, NaOEt and KOtBu.

In an embodiment, the base comprises at least one of NaOH, KOH, LiOH, NaHC03, Na2C03, KzC03, KCN, Et3N, NH3, HzN2H2, NaH, NaOMe, NaOEt or KOtBu.

In an embodiment, the base is KOH. In an embodiment, the base is LiOH. In an embodiment, the base is NaHCO₃. In an embodiment, the base is Na2C03.

In a preferred embodiment, the base is NaOH.

In some embodiments of the present invention, following the addition of the base, the solution is hold for some time prior to downstream processing.

In some embodiments of the present invention, the base treated solution is hold between a few seconds (e.g. 2 to 10 seconds) to about 1 day. Preferably the holding time is at least about 2, at least about 5, at least about 10, at least about 15, at least about 20, at least about 30, at least about 40, at least about 50, at least about 60, at least about 90, at least about 120 or at least about 160 minutes. Preferably the holding time is less than a week, however the holding time maybe longer.

Therefore, in certain embodiments, the holding time is between about 1 minute and 1 week. In certain embodiments, the holding time is between about 5 minute and 1 week. In certain embodiments, the holding time is between about 15 minute and 1 week. In certain embodiments, the holding time is between about 30 minute and 1 week. In certain embodiments, the holding time is between about 1 hour and 1 week. In certain embodiments, the holding time is between about 2 hours and 1 week. In certain embodiments, the holding time is between about 4 hours and 1 week. In certain embodiments, the holding time is between about 6 hours and 1 week. In certain embodiments, the holding time is between about 8 hours and 1 week. In certain embodiments, the holding time is between about 10 hours and 1 week. In certain embodiments, the holding time is between about 12 hours and 1 week. In certain embodiments, the holding time is between about 24 hours and 1 week. In certain embodiments, the holding time is between about 2 days and 1 week. In certain embodiments, the holding time is between about 4 days and 1 week. In certain embodiments, the holding time is between about 5 days and 1 week.

In some embodiments of the present invention, the holding time is between a few seconds (e.g. 1 to 10 seconds) and about one month. In some embodiments the holding time is between about 2 seconds and about two weeks. In some embodiments of the present invention, the holding time is between about 1 minute and about one week. In some embodiments the holding time is between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore, in certain embodiments, the holding time is between about 1 minute and one day. In certain embodiments, the holding time is between about 5 minute and one day. In certain embodiments, the holding time is between about 15 minute and one day. In certain embodiments, the holding time is between about 30 minute and one day. In certain embodiments, the holding time is between about 1 hour and one day. In certain embodiments, the holding time is between about 2 hours and one day. In certain embodiments, the holding time is between about 4 hours and one day. In certain embodiments, the holding time is between about 6 hours and one day. In certain embodiments, the holding time is between about 8 hours and one day. In certain embodiments, the holding time is between about 10 hours and one day. In certain embodiments, the holding time is between about 12 hours and one day.

In certain embodiments the holding time is between about 15 minutes and about 3 hours. In certain embodiments the holding time is between about 30 minutes and about 120 minutes.

Any number within any of the above ranges is contemplated as an embodiment of the disclosure.

In certain embodiments the holding time is about 2 seconds, about 10 seconds, about seconds, about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes, about 160 minutes, about 170 minutes, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, about 48 hours, about 3 days, about 4 days, about 5 days, about 6 days, about 7 days, about 8 days, about 9 days, about 10 days, about 11 days, about 12 days, about 13 days, about 14 days or about 15 days.

The holding time may be about 1 hour. The holding time may be about 2 hours. The holding time may be about 3 hours. The holding time may be about 4 hours. The holding time may be about 5 hours. The holding time may be about 6 hours. The holding time may be about 7 hours. The holding time may be about 8 hours. The holding time may be about 9 hours. The holding time may be about 10 hours. The holding time may be about 11 hours. The holding time may be about 12 hours. The holding time may be about 13 hours. The holding time may be about 14 hours. The holding time may be about 15 hours. The holding time may be about 16 hours. The holding time may be about 17 hours. The holding time may be about 18 hours. The holding time may be about 19 hours. The holding time may be about 20 hours. The holding time may be about 21 hours. The holding time may be about 22 hours. The holding time may be about 23 hours. The holding time may be about 24 hours.

Any number within any of the above ranges is contemplated as an embodiment of the disclosure.

In an embodiment, the optional holding step is conducted without agitation. In an embodiment, the optional holding step is conducted under agitation. In another embodiment, the optional holding step is conducted under gentle agitation. In another embodiment, the optional holding step is conducted under vigorous agitation.

In an embodiment of the present invention, the base treatment step is performed at a temperature between about 4° C. and about 30° C. In an embodiment, base treatment step is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C. In an embodiment, base treatment step is performed at a temperature of about 4° C. In an embodiment, base treatment step is performed at a temperature of about 5° C. In an embodiment, base treatment step is performed at a temperature of about 10° C. In an embodiment, base treatment step is performed at a temperature of about 15° C. In an embodiment, base treatment step is performed at a temperature of about 20° C. In an embodiment, base treatment step is performed at a temperature of about 25° C. In an embodiment, base treatment step is performed at a temperature of about 30° C. In a preferred embodiment, base treatment step is performed at a temperature of about 20° C.

1.3 Solid/Liquid Separation

The base treated material can be separated from the polysaccharide of interest by any suitable solid/liquid separation method.

Therefore, in an embodiment of the present invention, after base treatment, the suspension (as obtained at section 1.2 above) is clarified by decantation, sedimentation, filtration or centrifugation. In an embodiment the polysaccharide-containing solution is then collected for storage and/or additional processing.

In an embodiment of the present invention, after base treatment, the suspension (as obtained at section 1.2 above) is clarified by decantation. Decanters are used to separate liquids where there is a sufficient difference in density between the liquids for the floc to settle. In an operating decanter there will be three distinct zones: clear heavy liquid, separating dispersed liquid (the dispersion zone), and clear light liquid. To produce a clean solution, a small amount of solution must generally be left in the container. Decanters can be designed for continuous operation.

In an embodiment of the present invention, after base treatment, the suspension (as obtained at section 1.2 above) is clarified by sedimentation (settling). Sedimentation is the separation of suspended solid particles from a liquid mixture by gravity settling into a clear fluid and a slurry of higher solids content. Sedimentation can be done in a thickener, in a clarifier or in a classifier. Since thickening and clarification are relatively cheap processes when used for the treatment of large volumes of liquid, they can be used for pre-concentration of feeds to filtering.

In an embodiment of the present invention, after base treatment, the suspension (as obtained at section 1.2 above) is clarified by centrifugation. In an embodiment said centrifugation is continuous centrifugation. In an embodiment said centrifugation is bucket centrifugation. In an embodiment the polysaccharide-containing supernatant is then collected for storage and/or additional processing.

In some embodiments the suspension is centrifuged at about 1,000 g about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g. In some embodiments the suspension is centrifuged at about 8,000 g, about 9,000 g, about 10,000 g, about 11,000 g, about 12,000 g, about 13,000 g, about 14,000 g, about 15,000 g, about 16,000 g, about g, about 18,000 g, about 19,000 g, about 20,000 g or about 25,000 g. In some embodiments the suspension is centrifuged at about 1,000 g. In some embodiments the suspension is centrifuged at about 2,000 g. In some embodiments the suspension is centrifuged at about 5,000 g. In some embodiments the suspension is centrifuged at about 7,000 g. In some embodiments the suspension is centrifuged at about 8,000 g. In some embodiments the suspension is centrifuged at about 9,000 g. In some embodiments the suspension is centrifuged at about 10,000 g. In some embodiments the suspension is centrifuged at about 11,000 g. In some embodiments the suspension is centrifuged at about 12,000 g. In some embodiments the suspension is centrifuged at about 13,000 g. In some embodiments the suspension is centrifuged at about 14,000 g. In some embodiments the suspension is centrifuged at about 15,000 g. In some embodiments the suspension is centrifuged at about 20,000 g. In some embodiments the suspension is centrifuged at about 25,000 g. In some embodiments the suspension is centrifuged at about 30,000 g. In some embodiments the suspension is centrifuged at about 35,000 g. In some embodiments the suspension is centrifuged at about 40,000 g. In some embodiments the suspension is centrifuged at about 50,000 g. In some embodiments the suspension is centrifuged at about 75,000 g. In some embodiments the suspension is centrifuged at about 100,000 g. In some embodiments the suspension is centrifuged at about 120,000 g. In some embodiments the suspension is centrifuged at about 140,000 g. In some embodiments the suspension is centrifuged at about 160,000 g.

In some embodiments the suspension is centrifuged between about 5,000 g and about 25,000 g. In some embodiments the suspension is centrifuged between about 8,000 g and about 20,000 g. In some embodiments the suspension is centrifuged between about 10,000 g and about 15,000 g. In some embodiments the suspension is centrifuged between about 10,000 g and about 12,000 g.

Any number within any of the above ranges is contemplated as an embodiment of the disclosure.

In some embodiments the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes. Preferably the centrifugation time is less than 24 hours.

Therefore in certain embodiments, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360 minutes and about 380 minutes. In certain embodiments, the suspension is centrifuged during between about 5 and about 380 minutes. In certain embodiments, the suspension is centrifuged during between about and about 380 minutes. In certain embodiments, the suspension is centrifuged during between about 20 and about 380 minutes. In certain embodiments, the suspension is centrifuged during between about 30 and about 380 minutes. In certain embodiments, the suspension is centrifuged during between about 60 and about 380 minutes. In certain embodiments, the suspension is centrifuged during between about 90 and about 380 minutes. In certain embodiments, the suspension is centrifuged during between about 120 and about 380 minutes. In certain embodiments, the suspension is centrifuged during between about 150 and about 380 minutes. In certain embodiments, the suspension is centrifuged during between about 180 and about 380 minutes. In certain embodiments, the suspension is centrifuged during between about 210 and about 380 minutes. In certain embodiments, the suspension is centrifuged during between about 240 and about 380 minutes. In certain embodiments, the suspension is centrifuged during between about 270 and about 380 minutes. In certain embodiments, the suspension is centrifuged during between about 300 and about 380 minutes. In certain embodiments, the suspension is centrifuged during between about 330 and about 380 minutes. In certain embodiments, the suspension is centrifuged during between about 360 and about 380 minutes.

Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes. In certain embodiments the suspension is centrifuged during between about 5 minutes and about 3 hours. In certain the suspension is centrifuged during between about 5 minutes and about 120 minutes.

In certain embodiments, the suspension is centrifuged during between about 5 and about 160 minutes. In certain embodiments, the suspension is centrifuged during between about 10 and about 160 minutes. In certain embodiments, the suspension is centrifuged during between about 20 and about 160 minutes. In certain embodiments, the suspension is centrifuged during between about 30 and about 160 minutes. In certain embodiments, the suspension is centrifuged during between about 60 and about 160 minutes. In certain embodiments, the suspension is centrifuged during between about 90 and about 160 minutes. In certain embodiments, the suspension is centrifuged during between about 120 and about 160 minutes. In certain embodiments, the suspension is centrifuged during between about 150 and about 160 minutes. The suspension may be centrifuged during about 1 minute. The suspension may be centrifuged during about 5 minutes. The suspension may be centrifuged during about 10 minutes. The suspension may be centrifuged during about 15 minutes. The suspension may be centrifuged during about 20 minutes. The suspension may be centrifuged during about 25 minutes. The suspension may be centrifuged during about 30 minutes. The suspension may be centrifuged during about 40 minutes. The suspension may be centrifuged during about 50 minutes. The suspension may be centrifuged during about 1 hour. The suspension may be centrifuged during about 2 hours. The suspension may be centrifuged during about 3 hours. The suspension may be centrifuged during about 4 hours. The suspension may be centrifuged during about 5 hours. The suspension may be centrifuged during about 10 hours. The suspension may be centrifuged during about 15 hours. The suspension may be centrifuged during about hours. The suspension may be centrifuged during about 24 hours.

The suspension may be centrifuged during between about 2 minutes and about 60 minutes. The suspension may be centrifuged during between about 5 minutes and about 60 minutes. The suspension may be centrifuged during between about 10 minutes and about 60 minutes. The suspension may be centrifuged during between about 15 minutes and about 60 minutes. The suspension may be centrifuged during between about 20 minutes and about 60 minutes. The suspension may be centrifuged during between about 30 minutes and about 60 minutes. The suspension may be centrifuged during between about 45 minutes and about 60 minutes.

Any number within any of the above ranges is contemplated as an embodiment of the disclosure.

In an embodiment of the present invention, centrifugation is continuous centrifugation. In said embodiment, the feed rate can be of between of 50-5000 ml/min. In an embodiment, the feed rate can be of between of 100-4000 ml/min. In an embodiment, the feed rate can be of between of 150-3000 ml/min. In an embodiment, the feed rate can be of between of 200-2500 ml/min. In an embodiment, the feed rate can be of between of 250-2000 ml/min. In an embodiment, the feed rate can be of between of 300-1500 ml/min. In an embodiment, the feed rate can be of between of 300-1000 ml/min. In an embodiment, the feed rate can be of between of 200-1000 ml/min. In an embodiment, the feed rate can be of between of 200-1500 ml/min. In an embodiment, the feed rate can be of between of 400-1500 ml/min. In an embodiment, the feed rate can be of between of 500-1500 ml/min. In an embodiment, the feed rate can be of between of 500-1000 ml/min. In an embodiment, the feed rate can be of between of 500-2000 ml/min. In an embodiment, the feed rate can be of between of 500-2500 ml/min. In an embodiment, the feed rate can be of between of 1000-2500 ml/min.

In an embodiment, the feed rate can be of about 10 ml/min. In an embodiment, the feed rate can be of about 25 ml/min. In an embodiment, the feed rate can be of about 50 ml/min. In an embodiment, the feed rate can be of about 75 ml/min. In an embodiment, the feed rate can be of about 100 ml/min. In an embodiment, the feed rate can be of about 200 ml/min. In an embodiment, the feed rate can be of about 300 ml/min. In an embodiment, the feed rate can be of about 400 ml/min. In an embodiment, the feed rate can be of about 500 ml/min. In an embodiment, the feed rate can be of about 700 ml/min. In an embodiment, the feed rate can be of about 1000 ml/min. In an embodiment, the feed rate can be of about 1500 ml/min. In an embodiment, the feed rate can be of about 2000 ml/min. In an embodiment, the feed rate can be of about 2500 ml/min. In an embodiment, the feed rate can be of about 3000 ml/min. In an embodiment, the feed rate can be of about 3500 ml/min. In an embodiment, the feed rate can be of about 4000 ml/min. In an embodiment, the feed rate can be of about 5000 ml/min.

The solid/liquid separation methods described above can be used in a standalone format or in combination of two in any order, or in combination of three in any order.

1.4 Filtration (e.g. Depth Filtration)

Once the solution has been treated by the solid/liquid separation step of section 1.3 above, the Streptococcus pneumoniae serotype 3 polysaccharide containing solution (e.g. the supernatant) can optionally be further clarified.

In an embodiment, the solution is filtrated, thereby producing a further clarified solution. In an embodiment, the filtration is applied directly to the solution obtained by any of the method of section 1.3 above.

In an embodiment, the solution is treated by a filtration step selected from the group consisting of depth filtration, filtration through activated carbon and size filtration. In an embodiment, the solution is treated by depth filtration, filtration through activated carbon or size filtration. In an embodiment, the solution is treated by a depth filtration step.

Depth filters use a porous filtration medium to retain particles throughout the medium, rather than just on the surface of the medium. Due to the tortuous and channel-like nature of the filtration medium, the particles are retained throughout the medium within its structure, as opposed to on the surface.

In an embodiment, the solution is treated by a depth filtration step wherein the depth filter design is selected from the group consisting of cassettes, cartridges, deep bed (e.g. sand filter) and lenticular filters. In an embodiment, the solution is treated by a depth filtration step wherein the depth filter design is cassettes, cartridges, deep bed (e.g. sand filter) or lenticular filters.

In an embodiment, the solution is treated by a depth filtration step wherein the depth filter has a nominal retention range of between about 0.01-100 micron. In an embodiment, the solution is treated by a depth filtration step wherein the depth filter has a nominal retention range of between about 0.05-100 micron. In an embodiment, the solution is treated by a depth filtration step wherein the depth filter has a nominal retention range of between about 0.1-100 micron. In an embodiment, the solution is treated by a depth filtration step wherein the depth filter has a nominal retention range of between about 0.2-100 micron. In an embodiment, the solution is treated by a depth filtration step wherein the depth filter has a nominal retention range of between about 0.5-100 micron. In an embodiment, the solution is treated by a depth filtration step wherein the depth filter has a nominal retention range of between about 0.8-100 micron. In an embodiment, the solution is treated by a depth filtration step wherein the depth filter has a nominal retention range of between about 1-100 micron. In an embodiment, the solution is treated by a depth filtration step wherein the depth filter has a nominal retention range of between about 1.25-100 micron. In an embodiment, the solution is treated by a depth filtration step wherein the depth filter has a nominal retention range of between about 1.5-100 micron. In an embodiment, the solution is treated by a depth filtration step wherein the depth filter has a nominal retention range of between about 2-100 micron. In an embodiment, the solution is treated by a depth filtration step wherein the depth filter has a nominal retention range of between about 3-100 micron. In an embodiment, the solution is treated by a depth filtration step wherein the depth filter has a nominal retention range of between about 5-100 micron. In an embodiment, the solution is treated by a depth filtration step wherein the depth filter has a nominal retention range of between about 8-100 micron. In an embodiment, the solution is treated by a depth filtration step wherein the depth filter has a nominal retention range of between about 10-100 micron. In an embodiment, the solution is treated by a depth filtration step wherein the depth filter has a nominal retention range of between about 15-100 micron. In an embodiment, the solution is treated by a depth filtration step wherein the depth filter has a nominal retention range of between about 20-100 micron. In an embodiment, the solution is treated by a depth filtration step wherein the depth filter has a nominal retention range of between about 30-100 micron. In an embodiment, the solution is treated by a depth filtration step wherein the depth filter has a nominal retention range of between about 40-100 micron. In an embodiment, the solution is treated by a depth filtration step wherein the depth filter has a nominal retention range of between about 50-100 micron. In an embodiment, the solution is treated by a depth filtration step wherein the depth filter has a nominal retention range of between about 75-100 micron.

In an embodiment, the solution is treated by a depth filtration step wherein the depth filter has a nominal retention range of between about 0.01-75 micron. In an embodiment, the solution is treated by a depth filtration step wherein the depth filter has a nominal retention range of between about 0.05-75 micron. In an embodiment, the solution is treated by a depth filtration step wherein the depth filter has a nominal retention range of between about 0.1-75 micron. In an embodiment, the solution is treated by a depth filtration step wherein the depth filter has a nominal retention range of between about 0.2-75 micron. In an embodiment, the solution is treated by a depth filtration step wherein the depth filter has a nominal retention range of between about 0.5-75 micron. In an embodiment, the solution is treated by a depth filtration step wherein the depth filter has a nominal retention range of between about 0.8-75 micron. In an embodiment, the solution is treated by a depth filtration step wherein the depth filter has a nominal retention range of between about 1-75 micron. In an embodiment, the solution is treated by a depth filtration step wherein the depth filter has a nominal retention range of between about 1.25-75 micron. In an embodiment, the solution is treated by a depth filtration step wherein the depth filter has a nominal retention range of between about 1.5-75 micron. In an embodiment, the solution is treated by a depth filtration step wherein the depth filter has a nominal retention range of between about 2-75 micron. In an embodiment, the solution is treated by a depth filtration step wherein the depth filter has a nominal retention range of between about 3-75 micron. In an embodiment, the solution is treated by a depth filtration step wherein the depth filter has a nominal retention range of between about 5-75 micron. In an embodiment, the solution is treated by a depth filtration step wherein the depth filter has a nominal retention range of between about 8-75 micron. In an embodiment, the solution is treated by a depth filtration step wherein the depth filter has a nominal retention range of between about 10-75 micron. In an embodiment, the solution is treated by a depth filtration step wherein the depth filter has a nominal retention range of between about 15-75 micron. In an embodiment, the solution is treated by a depth filtration step wherein the depth filter has a nominal retention range of between about 20-75 micron. In an embodiment, the solution is treated by a depth filtration step wherein the depth filter has a nominal retention range of between about 30-75 micron. In an embodiment, the solution is treated by a depth filtration step wherein the depth filter has a nominal retention range of between about 40-75 micron. In an embodiment, the solution is treated by a depth filtration step wherein the depth filter has a nominal retention range of between about 50-75 micron.

In an embodiment, the solution is treated by a depth filtration step wherein the depth filter has a nominal retention range of between about 0.01-50 micron. In an embodiment, the solution is treated by a depth filtration step wherein the depth filter has a nominal retention range of between about 0.05-50 micron. In an embodiment, the solution is treated by a depth filtration step wherein the depth filter has a nominal retention range of between about 0.1-50 micron. In an embodiment, the solution is treated by a depth filtration step wherein the depth filter has a nominal retention range of between about 0.2-50 micron. In an embodiment, the solution is treated by a depth filtration step wherein the depth filter has a nominal retention range of between about 0.5-50 micron. In an embodiment, the solution is treated by a depth filtration step wherein the depth filter has a nominal retention range of between about 0.8-50 micron. In an embodiment, the solution is treated by a depth filtration step wherein the depth filter has a nominal retention range of between about 1-50 micron. In an embodiment, the solution is treated by a depth filtration step wherein the depth filter has a nominal retention range of between about 1.25-50 micron. In an embodiment, the solution is treated by a depth filtration step wherein the depth filter has a nominal retention range of between about 1.5-50 micron. In an embodiment, the solution is treated by a depth filtration step wherein the depth filter has a nominal retention range of between about 2-50 micron. In an embodiment, the solution is treated by a depth filtration step wherein the depth filter has a nominal retention range of between about 3-50 micron. In an embodiment, the solution is treated by a depth filtration step wherein the depth filter has a nominal retention range of between about 5-50 micron. In an embodiment, the solution is treated by a depth filtration step wherein the depth filter has a nominal retention range of between about 8-50 micron. In an embodiment, the solution is treated by a depth filtration step wherein the depth filter has a nominal retention range of between about 10-50 micron. In an embodiment, the solution is treated by a depth filtration step wherein the depth filter has a nominal retention range of between about 15-50 micron. In an embodiment, the solution is treated by a depth filtration step wherein the depth filter has a nominal retention range of between about 20-50 micron. In an embodiment, the solution is treated by a depth filtration step wherein the depth filter has a nominal retention range of between about 30-50 micron. In an embodiment, the solution is treated by a depth filtration step wherein the depth filter has a nominal retention range of between about 40-50 micron.

In an embodiment, the solution is treated by a depth filtration step wherein the depth filter has a nominal retention range of between about 0.01-25 micron. In an embodiment, the solution is treated by a depth filtration step wherein the depth filter has a nominal retention range of between about 0.05-25 micron. In an embodiment, the solution is treated by a depth filtration step wherein the depth filter has a nominal retention range of between about 0.1-25 micron. In an embodiment, the solution is treated by a depth filtration step wherein the depth filter has a nominal retention range of between about 0.2-25 micron. In an embodiment, the solution is treated by a depth filtration step wherein the depth filter has a nominal retention range of between about 0.5-25 micron. In an embodiment, the solution is treated by a depth filtration step wherein the depth filter has a nominal retention range of between about 0.8-25 micron. In an embodiment, the solution is treated by a depth filtration step wherein the depth filter has a nominal retention range of between about 1-25 micron. In an embodiment, the solution is treated by a depth filtration step wherein the depth filter has a nominal retention range of between about 1.25-25 micron. In an embodiment, the solution is treated by a depth filtration step wherein the depth filter has a nominal retention range of between about 1.5-25 micron. In an embodiment, the solution is treated by a depth filtration step wherein the depth filter has a nominal retention range of between about 2-25 micron. In an embodiment, the solution is treated by a depth filtration step wherein the depth filter has a nominal retention range of between about 3-25 micron. In an embodiment, the solution is treated by a depth filtration step wherein the depth filter has a nominal retention range of between about 5-micron. In an embodiment, the solution is treated by a depth filtration step wherein the depth filter has a nominal retention range of between about 8-25 micron. In an embodiment, the solution is treated by a depth filtration step wherein the depth filter has a nominal retention range of between about 10-25 micron. In an embodiment, the solution is treated by a depth filtration step wherein the depth filter has a nominal retention range of between about 15-25 micron. In an embodiment, the solution is treated by a depth filtration step wherein the depth filter has a nominal retention range of between about 20-25 micron.

In an embodiment, the solution is treated by a depth filtration step wherein the depth filter has a nominal retention range of between about 0.01-10 micron. In an embodiment, the solution is treated by a depth filtration step wherein the depth filter has a nominal retention range of between about 0.05-10 micron. In an embodiment, the solution is treated by a depth filtration step wherein the depth filter has a nominal retention range of between about 0.1-10 micron. In an embodiment, the solution is treated by a depth filtration step wherein the depth filter has a nominal retention range of between about 0.2-25 micron. In an embodiment, the solution is treated by a depth filtration step wherein the depth filter has a nominal retention range of between about 0.5-10 micron. In an embodiment, the solution is treated by a depth filtration step wherein the depth filter has a nominal retention range of between about 0.8-10 micron. In an embodiment, the solution is treated by a depth filtration step wherein the depth filter has a nominal retention range of between about 1-10 micron. In an embodiment, the solution is treated by a depth filtration step wherein the depth filter has a nominal retention range of between about 1.25-10 micron. In an embodiment, the solution is treated by a depth filtration step wherein the depth filter has a nominal retention range of between about 1.5-10 micron. In an embodiment, the solution is treated by a depth filtration step wherein the depth filter has a nominal retention range of between about 2-10 micron. In an embodiment, the solution is treated by a depth filtration step wherein the depth filter has a nominal retention range of between about 3-10 micron. In an embodiment, the solution is treated by a depth filtration step wherein the depth filter has a nominal retention range of between about 5-micron. In an embodiment, the solution is treated by a depth filtration step wherein the depth filter has a nominal retention range of between about 8-10 micron.

In an embodiment, the solution is treated by a depth filtration step wherein the depth filter has a nominal retention range of between about 0.01-8 micron. In an embodiment, the solution is treated by a depth filtration step wherein the depth filter has a nominal retention range of between about 0.05-8 micron. In an embodiment, the solution is treated by a depth filtration step wherein the depth filter has a nominal retention range of between about 0.1-8 micron. In an embodiment, the solution is treated by a depth filtration step wherein the depth filter has a nominal retention range of between about 0.2-8 micron. In an embodiment, the solution is treated by a depth filtration step wherein the depth filter has a nominal retention range of between about 0.5-8 micron. In an embodiment, the solution is treated by a depth filtration step wherein the depth filter has a nominal retention range of between about 0.8-8 micron. In an embodiment, the solution is treated by a depth filtration step wherein the depth filter has a nominal retention range of between about 1-8 micron. In an embodiment, the solution is treated by a depth filtration step wherein the depth filter has a nominal retention range of between about 1.25-8 micron. In an embodiment, the solution is treated by a depth filtration step wherein the depth filter has a nominal retention range of between about 1.5-8 micron. In an embodiment, the solution is treated by a depth filtration step wherein the depth filter has a nominal retention range of between about 2-8 micron. In an embodiment, the solution is treated by a depth filtration step wherein the depth filter has a nominal retention range of between about 3-8 micron. In an embodiment, the solution is treated by a depth filtration step wherein the depth filter has a nominal retention range of between about 5-8 micron.

In an embodiment, the solution is treated by a depth filtration step wherein the depth filter has a nominal retention range of between about 0.01-5 micron. In an embodiment, the solution is treated by a depth filtration step wherein the depth filter has a nominal retention range of between about 0.05-5 micron. In an embodiment, the solution is treated by a depth filtration step wherein the depth filter has a nominal retention range of between about 0.1-5 micron. In an embodiment, the solution is treated by a depth filtration step wherein the depth filter has a nominal retention range of between about 0.2-5 micron. In an embodiment, the solution is treated by a depth filtration step wherein the depth filter has a nominal retention range of between about 0.5-5 micron. In an embodiment, the solution is treated by a depth filtration step wherein the depth filter has a nominal retention range of between about 0.8-5 micron. In an embodiment, the solution is treated by a depth filtration step wherein the depth filter has a nominal retention range of between about 1-micron. In an embodiment, the solution is treated by a depth filtration step wherein the depth filter has a nominal retention range of between about 1.25-5 micron. In an embodiment, the solution is treated by a depth filtration step wherein the depth filter has a nominal retention range of between about 1.5-5 micron. In an embodiment, the solution is treated by a depth filtration step wherein the depth filter has a nominal retention range of between about 2-5 micron. In an embodiment, the solution is treated by a depth filtration step wherein the depth filter has a nominal retention range of between about 3-5 micron.

In an embodiment, the solution is treated by a depth filtration step wherein the depth filter has a nominal retention range of between about 0.01-2 micron. In an embodiment, the solution is treated by a depth filtration step wherein the depth filter has a nominal retention range of between about 0.05-2 micron. In an embodiment, the solution is treated by a depth filtration step wherein the depth filter has a nominal retention range of between about 0.1-2 micron. In an embodiment, the solution is treated by a depth filtration step wherein the depth filter has a nominal retention range of between about 0.2-2 micron. In an embodiment, the solution is treated by a depth filtration step wherein the depth filter has a nominal retention range of between about 0.5-2 micron. In an embodiment, the solution is treated by a depth filtration step wherein the depth filter has a nominal retention range of between about 0.8-2 micron. In an embodiment, the solution is treated by a depth filtration step wherein the depth filter has a nominal retention range of between about 1-2 micron. In an embodiment, the solution is treated by a depth filtration step wherein the depth filter has a nominal retention range of between about 1.25-2 micron. In an embodiment, the solution is treated by a depth filtration step wherein the depth filter has a nominal retention range of between about 1.5-2 micron.

In an embodiment, the solution is treated by a depth filtration step wherein the depth filter has a nominal retention range of between about 0.01-1 micron. In an embodiment, the solution is treated by a depth filtration step wherein the depth filter has a nominal retention range of between about 0.05-1 micron. In an embodiment, the solution is treated by a depth filtration step wherein the depth filter has a nominal retention range of between about 0.1-1 micron. In an embodiment, the solution is treated by a depth filtration step wherein the depth filter has a nominal retention range of between about 0.2-1 micron. In an embodiment, the solution is treated by a depth filtration step wherein the depth filter has a nominal retention range of between about 0.5-1 micron. In an embodiment, the solution is treated by a depth filtration step wherein the depth filter has a nominal retention range of between about 0.8-1 micron.

In an embodiment, the solution is treated by a depth filtration step wherein the depth filter has a nominal retention range of between about 0.05-50 micron. In an embodiment, the solution is treated by a depth filtration step wherein the depth filter has a nominal retention range of between about 0.1-25 micron. In an embodiment, the solution is treated by a depth filtration step wherein the depth filter has a nominal retention range of between about 0.2-10, micron. In an embodiment, the solution is treated by a depth filtration step wherein the depth filter has a nominal retention range of between about 0.1-10 micron. In an embodiment, the solution is treated by a depth filtration step wherein the depth filter has a nominal retention range of between about 0.2-5 micron. In an embodiment, the solution is treated by a depth filtration step wherein the depth filter has a nominal retention range of between about 0.25-1 micron.

Any number within any of the above ranges is contemplated as an embodiment of the disclosure.

In an embodiment, the solution is treated by a depth filtration step wherein the depth filter has a filter capacity of 1-2500 L/m². In an embodiment, the solution is treated by a depth filtration step wherein the depth filter has a filter capacity of 10-2500 L/m². In an embodiment, the solution is treated by a depth filtration step wherein the depth filter has a filter capacity of 25-2500 L/m². In an embodiment, the solution is treated by a depth filtration step wherein the depth filter has a filter capacity of 50-2500 L/m². In an embodiment, the solution is treated by a depth filtration step wherein the depth filter has a filter capacity of 100-2500 L/m². In an embodiment, the solution is treated by a depth filtration step wherein the depth filter has a filter capacity of 200-2500 L/m². In an embodiment, the solution is treated by a depth filtration step wherein the depth filter has a filter capacity of 400-2500 L/m². In an embodiment, the solution is treated by a depth filtration step wherein the depth filter has a filter capacity of 500-2500 L/m². In an embodiment, the solution is treated by a depth filtration step wherein the depth filter has a filter capacity of 750-2500 L/m². In an embodiment, the solution is treated by a depth filtration step wherein the depth filter has a filter capacity of 1000-2500 L/m². In an embodiment, the solution is treated by a depth filtration step wherein the depth filter has a filter capacity of 1500-2500 L/m². In an embodiment, the solution is treated by a depth filtration step wherein the depth filter has a filter capacity of 2000-2500 L/m².

In an embodiment, the solution is treated by a depth filtration step wherein the depth filter has a filter capacity of 1-1000 L/m². In an embodiment, the solution is treated by a depth filtration step wherein the depth filter has a filter capacity of 5-1000 L/m². In an embodiment, the solution is treated by a depth filtration step wherein the depth filter has a filter capacity of 10-1000 L/m². In an embodiment, the solution is treated by a depth filtration step wherein the depth filter has a filter capacity of 50-1000 L/m². In an embodiment, the solution is treated by a depth filtration step wherein the depth filter has a filter capacity of 100-1000 L/m². In an embodiment, the solution is treated by a depth filtration step wherein the depth filter has a filter capacity of 150-1000 L/m². In an embodiment, the solution is treated by a depth filtration step wherein the depth filter has a filter capacity of 200-1000 L/m². In an embodiment, the solution is treated by a depth filtration step wherein the depth filter has a filter capacity of 250-1000 L/m². In an embodiment, the solution is treated by a depth filtration step wherein the depth filter has a filter capacity of 500-1000 L/m². In an embodiment, the solution is treated by a depth filtration step wherein the depth filter has a filter capacity of 750-1000 L/m².

In an embodiment, the solution is treated by a depth filtration step wherein the depth filter has a filter capacity of 1-750 L/m². In an embodiment, the solution is treated by a depth filtration step wherein the depth filter has a filter capacity of 5-750 L/m². In an embodiment, the solution is treated by a depth filtration step wherein the depth filter has a filter capacity of 10-750 L/m². In an embodiment, the solution is treated by a depth filtration step wherein the depth filter has a filter capacity of 50-750 L/m². In an embodiment, the solution is treated by a depth filtration step wherein the depth filter has a filter capacity of 100-750 L/m². In an embodiment, the solution is treated by a depth filtration step wherein the depth filter has a filter capacity of 150-750 L/m². In an embodiment, the solution is treated by a depth filtration step wherein the depth filter has a filter capacity of 200-750 L/m². In an embodiment, the solution is treated by a depth filtration step wherein the depth filter has a filter capacity of 250-750 L/m². In an embodiment, the solution is treated by a depth filtration step wherein the depth filter has a filter capacity of 500-750 L/m².

In an embodiment, the solution is treated by a depth filtration step wherein the depth filter has a filter capacity of 1-500 L/m². In an embodiment, the solution is treated by a depth filtration step wherein the depth filter has a filter capacity of 5-500 L/m². In an embodiment, the solution is treated by a depth filtration step wherein the depth filter has a filter capacity of 10-500 L/m². In an embodiment, the solution is treated by a depth filtration step wherein the depth filter has a filter capacity of 50-500 L/m². In an embodiment, the solution is treated by a depth filtration step wherein the depth filter has a filter capacity of 100-500 L/m². In an embodiment, the solution is treated by a depth filtration step wherein the depth filter has a filter capacity of 150-500 L/m². In an embodiment, the solution is treated by a depth filtration step wherein the depth filter has a filter capacity of 200-500 L/m². In an embodiment, the solution is treated by a depth filtration step wherein the depth filter has a filter capacity of 250-500 L/m². In an embodiment, the solution is treated by a depth filtration step wherein the depth filter has a filter capacity of 300-500 L/m². In an embodiment, the solution is treated by a depth filtration step wherein the depth filter has a filter capacity of 400-500 L/m².

In an embodiment, the solution is treated by a depth filtration step wherein the depth filter has a filter capacity of 1-300 L/m². In an embodiment, the solution is treated by a depth filtration step wherein the depth filter has a filter capacity of 5-300 L/m². In an embodiment, the solution is treated by a depth filtration step wherein the depth filter has a filter capacity of 10-300 L/m². In an embodiment, the solution is treated by a depth filtration step wherein the depth filter has a filter capacity of 50-300 L/m². In an embodiment, the solution is treated by a depth filtration step wherein the depth filter has a filter capacity of 100-300 L/m². In an embodiment, the solution is treated by a depth filtration step wherein the depth filter has a filter capacity of 150-300 L/m². In an embodiment, the solution is treated by a depth filtration step wherein the depth filter has a filter capacity of 200-300 L/m². In an embodiment, the solution is treated by a depth filtration step wherein the depth filter has a filter capacity of 250-300 L/m².

In an embodiment, the solution is treated by a depth filtration step wherein the depth filter has a filter capacity of 1-200 L/m². In an embodiment, the solution is treated by a depth filtration step wherein the depth filter has a filter capacity of 5-200 L/m². In an embodiment, the solution is treated by a depth filtration step wherein the depth filter has a filter capacity of 10-200 L/m². In an embodiment, the solution is treated by a depth filtration step wherein the depth filter has a filter capacity of 50-200 L/m². In an embodiment, the solution is treated by a depth filtration step wherein the depth filter has a filter capacity of 100-200 L/m². In an embodiment, the solution is treated by a depth filtration step wherein the depth filter has a filter capacity of 150-200 L/m².

In an embodiment, the solution is treated by a depth filtration step wherein the depth filter has a filter capacity of 1-100 L/m². In an embodiment, the solution is treated by a depth filtration step wherein the depth filter has a filter capacity of 5-100 L/m². In an embodiment, the solution is treated by a depth filtration step wherein the depth filter has a filter capacity of 10-100 L/m². In an embodiment, the solution is treated by a depth filtration step wherein the depth filter has a filter capacity of 50-100 L/m². In an embodiment, the solution is treated by a depth filtration step wherein the depth filter has a filter capacity of 75-100 L/m².

In an embodiment, the solution is treated by a depth filtration step wherein the depth filter has a filter capacity of 1-50 L/m². In an embodiment, the solution is treated by a depth filtration step wherein the depth filter has a filter capacity of 5-50 L/m². In an embodiment, the solution is treated by a depth filtration step wherein the depth filter has a filter capacity of 10-50 L/m². In an embodiment, the solution is treated by a depth filtration step wherein the depth filter has a filter capacity of 25-50 L/m².

Any whole number integer within any of the above ranges is contemplated as an embodiment of the disclosure.

In an embodiment, the solution is treated by a depth filtration step wherein the feed rate is between 1-1000 LMH (liters/m²/hour). In an embodiment, the solution is treated by a depth filtration step wherein the feed rate is between 10-1000 LMH. In an embodiment, the solution is treated by a depth filtration step wherein the feed rate is between 25-1000 LMH. In an embodiment, the solution is treated by a depth filtration step wherein the feed rate is between 50-1000 LMH. In an embodiment, the solution is treated by a depth filtration step wherein the feed rate is between 100-1000 LMH. In an embodiment, the solution is treated by a depth filtration step wherein the feed rate is between 125-1000 LMH. In an embodiment, the solution is treated by a depth filtration step wherein the feed rate is between 150-1000 LMH. In an embodiment, the solution is treated by a depth filtration step wherein the feed rate is between 200-1000 LMH. In an embodiment, the solution is treated by a depth filtration step wherein the feed rate is between 300-1000 LMH. In an embodiment, the solution is treated by a depth filtration step wherein the feed rate is between 400-1000 LMH. In an embodiment, the solution is treated by a depth filtration step wherein the feed rate is between 500-1000 LMH. In an embodiment, the solution is treated by a depth filtration step wherein the feed rate is between 600-1000 LMH. In an embodiment, the solution is treated by a depth filtration step wherein the feed rate is between 700-1000 LMH. In an embodiment, the solution is treated by a depth filtration step wherein the feed rate is between 800-1000 LMH. In an embodiment, the solution is treated by a depth filtration step wherein the feed rate is between 900-1000 LMH.

In an embodiment, the solution is treated by a depth filtration step wherein the feed rate is between 1-500 LMH. In an embodiment, the solution is treated by a depth filtration step wherein the feed rate is between 10-500 LMH. In an embodiment, the solution is treated by a depth filtration step wherein the feed rate is between 25-500 LMH. In an embodiment, the solution is treated by a depth filtration step wherein the feed rate is between 50-500 LMH. In an embodiment, the solution is treated by a depth filtration step wherein the feed rate is between 100-500 LMH. In an embodiment, the solution is treated by a depth filtration step wherein the feed rate is between 125-500 LMH. In an embodiment, the solution is treated by a depth filtration step wherein the feed rate is between 200-500 LMH. In an embodiment, the solution is treated by a depth filtration step wherein the feed rate is between 300-500 LMH. In an embodiment, the solution is treated by a depth filtration step wherein the feed rate is between 400-500 LMH.

In an embodiment, the solution is treated by a depth filtration step wherein the feed rate is between 1-400 LMH. In an embodiment, the solution is treated by a depth filtration step wherein the feed rate is between 10-400 LMH. In an embodiment, the solution is treated by a depth filtration step wherein the feed rate is between 25-400 LMH. In an embodiment, the solution is treated by a depth filtration step wherein the feed rate is between 50-400 LMH. In an embodiment, the solution is treated by a depth filtration step wherein the feed rate is between 100-400 LMH. In an embodiment, the solution is treated by a depth filtration step wherein the feed rate is between 25-400 LMH. In an embodiment, the solution is treated by a depth filtration step wherein the feed rate is between 150-400 LMH. In an embodiment, the solution is treated by a depth filtration step wherein the feed rate is between 200-400 LMH. In an embodiment, the solution is treated by a depth filtration step wherein the feed rate is between 250-400 LMH. In an embodiment, the solution is treated by a depth filtration step wherein the feed rate is between 300-400 LMH.

In an embodiment, the solution is treated by a depth filtration step wherein the feed rate is between 1-250 LMH. In an embodiment, the solution is treated by a depth filtration step wherein the feed rate is between 10-250 LMH. In an embodiment, the solution is treated by a depth filtration step wherein the feed rate is between 25-250 LMH. In an embodiment, the solution is treated by a depth filtration step wherein the feed rate is between 50-250 LMH. In an embodiment, the solution is treated by a depth filtration step wherein the feed rate is between 100-250 LMH. In an embodiment, the solution is treated by a depth filtration step wherein the feed rate is between 125-250 LMH. In an embodiment, the solution is treated by a depth filtration step wherein the feed rate is between 150-250 LMH. In an embodiment, the solution is treated by a depth filtration step wherein the feed rate is between 200-250 LMH.

Any number within any of the above ranges is contemplated as an embodiment of the disclosure.

In an embodiment, the solution is treated by a depth filtration step wherein the feed rate is about 1 LMH. In an embodiment, the solution is treated by a depth filtration step wherein the feed rate is about 2 LMH. In an embodiment, the solution is treated by a depth filtration step wherein the feed rate is about 5 LMH. In an embodiment, the solution is treated by a depth filtration step wherein the feed rate is about 10 LMH. In an embodiment, the solution is treated by a depth filtration step wherein the feed rate is about LMH. In an embodiment, the solution is treated by a depth filtration step wherein the feed rate is about 50 LMH. In an embodiment, the solution is treated by a depth filtration step wherein the feed rate is about 75 LMH. In an embodiment, the solution is treated by a depth filtration step wherein the feed rate is about 100 LMH. In an embodiment, the solution is treated by a depth filtration step wherein the feed rate is about 125 LMH. In an embodiment, the solution is treated by a depth filtration step wherein the feed rate is about 150 LMH. In an embodiment, the solution is treated by a depth filtration step wherein the feed rate is about 175 LMH. In an embodiment, the solution is treated by a depth filtration step wherein the feed rate is about 200 LMH. In an embodiment, the solution is treated by a depth filtration step wherein the feed rate is about 250 LMH. In an embodiment, the solution is treated by a depth filtration step wherein the feed rate is about 300 LMH. In an embodiment, the solution is treated by a depth filtration step wherein the feed rate is about 350 LMH. In an embodiment, the solution is treated by a depth filtration step wherein the feed rate is about 400 LMH. In an embodiment, the solution is treated by a depth filtration step wherein the feed rate is about 500 LMH. In an embodiment, the solution is treated by a depth filtration step wherein the feed rate is about 750 LMH. In an embodiment, the solution is treated by a depth filtration step wherein the feed rate is about 1000 LMH.

1.5 Ultrafiltration and/or Diafiltration

The solution obtained above can optionally be further clarified by Ultrafiltration and/or Diafiltration. In an embodiment of the present invention, the solution obtained after base treatment by any of the method of section 1.2 above is clarified by Ultrafiltration and/or Diafiltration. In an embodiment the polysaccharide-containing solution is then collected for storage and/or additional processing.

In another embodiment of the present invention, the solution treated by the solid/liquid separation step of section 1.3 above is further clarified by Ultrafiltration and/or Diafiltration. In an embodiment, the solution treated by the solid/liquid separation step of section 1.3 is further filtrated as disclosed at section 1.4 above and is then further clarified by Ultrafiltration and/or Diafiltration.

Ultrafiltration (UF) is a process for concentrating a dilute product stream. UF separates molecules in solution based on the membrane pore size or molecular weight cutoff (MWCO).

In an embodiment of the present invention, the solution (i.e. the base treated solution obtained by any of the method of section 1.2 above, the solution treated by the solid/liquid separation step of section 1.3 above or the solution treated by the solid/liquid separation step of section 1.3 further filtrated as disclosed at section 1.4 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa. In an embodiment the molecular weight cut off of the membrane is in the range of between about 10 kDa-1000 kDa. In an embodiment the molecular weight cut off of the membrane is in the range of between about 20 kDa-1000 kDa. In an embodiment the molecular weight cut off of the membrane is in the range of between about 30 kDa-1000 kDa. In an embodiment the molecular weight cut off of the membrane is in the range of between about 100 kDa-1000 kDa. In an embodiment the molecular weight cut off of the membrane is in the range of between about 250 kDa-1000 kDa. In an embodiment the molecular weight cut off of the membrane is in the range of between about 500 kDa-1000 kDa. In an embodiment the molecular weight cut off of the membrane is in the range of between about 750 kDa-1000 kDa.

In an embodiment the molecular weight cut off of the membrane is in the range of between about 5 kDa-500 kDa. In an embodiment the molecular weight cut off of the membrane is in the range of between about 10 kDa-500 kDa. In an embodiment the molecular weight cut off of the membrane is in the range of between about 20 kDa-500 kDa. In an embodiment the molecular weight cut off of the membrane is in the range of between about 30 kDa-500 kDa. In an embodiment the molecular weight cut off of the membrane is in the range of between about 50 kDa-500 kDa. In an embodiment the molecular weight cut off of the membrane is in the range of between about 100 kDa-500 kDa. In an embodiment the molecular weight cut off of the membrane is in the range of between about 250 kDa-500 kDa. In an embodiment the molecular weight cut off of the membrane is in the range of between about 400 kDa-500 kDa.

In an embodiment the molecular weight cut off of the membrane is in the range of between about 5 kDa-100 kDa. In an embodiment the molecular weight cut off of the membrane is in the range of between about 10 kDa-100 kDa. In an embodiment the molecular weight cut off of the membrane is in the range of between about 20 kDa-100 kDa. In an embodiment the molecular weight cut off of the membrane is in the range of between about 30 kDa-100 kDa. In an embodiment the molecular weight cut off of the membrane is in the range of between about 40 kDa-100 kDa. In an embodiment the molecular weight cut off of the membrane is in the range of between about 50 kDa-100 kDa. In an embodiment the molecular weight cut off of the membrane is in the range of between about 75 kDa-100 kDa.

In an embodiment the molecular weight cut off of the membrane is about 5 kDa. In an embodiment the molecular weight cut off of the membrane is about 10 kDa. In an embodiment the molecular weight cut off of the membrane is about 20 kDa. In an embodiment the molecular weight cut off of the membrane is about 30 kDa. In an embodiment the molecular weight cut off of the membrane is about 40 kDa. In an embodiment the molecular weight cut off of the membrane is about 50 kDa. In an embodiment the molecular weight cut off of the membrane is about 70 kDa. In an embodiment the molecular weight cut off of the membrane is about 100 kDa. In an embodiment the molecular weight cut off of the membrane is about 150 kDa. In an embodiment the molecular weight cut off of the membrane is about 200 kDa. In an embodiment the molecular weight cut off of the membrane is about 250 kDa. In an embodiment the molecular weight cut off of the membrane is about 400 kDa. In an embodiment the molecular weight cut off of the membrane is about 500 kDa. In an embodiment the molecular weight cut off of the membrane is about 750 kDa. In an embodiment the molecular weight cut off of the membrane is about 1000 kDa.

In an embodiment, the concentration factor of the ultrafiltration step is from about 1.5 to 10. In an embodiment, the concentration factor is from about 2 to 8. In an embodiment, the concentration factor is from about 2 to 5.

In an embodiment, the concentration factor is about 1.5 In an embodiment, the concentration factor is about 2.0. In an embodiment, the concentration factor is about 3.0. In an embodiment, the concentration factor is about 4.0. In an embodiment, the concentration factor is about 5.0. In an embodiment, the concentration factor is about 6.0. In an embodiment, the concentration factor is about 7.0. In an embodiment, the concentration factor is about 8.0. In an embodiment, the concentration factor is about 9.0. In an embodiment, the concentration factor is about 10.0.

In an embodiment of the present invention, the solution (i.e. the base treated solution obtained by any of the method of section 1.2 above, the solution treated by the solid/liquid separation step of section 1.3 above or the solution treated by the solid/liquid separation step of section 1.3 further filtrated as disclosed at section 1.4 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the group consisting of magnesium chloride, potassium chloride, sodium chloride and a combination thereof. In some embodiments, the salt comprises at least one of magnesium chloride, potassium chloride, sodium chloride or a combination thereof. In one particular embodiment, the salt is magnesium chloride. In one particular embodiment, the salt is potassium chloride. In one particular embodiment, the salt is sodium chloride. In one particular embodiment, the salt is sodium chloride. In one embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM. In one particular embodiment, the replacement solution is sodium chloride at about 40 mM, about 45 mM, about 50 mM, about 55 mM, about 60 mM, about 65 mM, about 70 mM, about 80 mM, about 90 mM or about 100 mM. In one embodiment, the replacement solution is sodium chloride at about 1 mM. In one embodiment, the replacement solution is sodium chloride at about 5 mM. In one embodiment, the replacement solution is sodium chloride at about 10 mM. In one embodiment, the replacement solution is sodium chloride at about 20 mM. In one embodiment, the replacement solution is sodium chloride at about 30 mM. In one embodiment, the replacement solution is sodium chloride at about 40 mM. In one embodiment, the replacement solution is sodium chloride at about 50 mM. In one embodiment, the replacement solution is sodium chloride at about 60 mM. In one embodiment, the replacement solution is sodium chloride at about 70 mM. In one embodiment, the replacement solution is sodium chloride at about 80 mM. In one embodiment, the replacement solution is sodium chloride at about 90 mM. In one embodiment, the replacement solution is sodium chloride at about 100 mM. In one embodiment, the replacement solution is sodium chloride at about 110 mM. In one embodiment, the replacement solution is sodium chloride at about 120 mM. In one embodiment, the replacement solution is sodium chloride at about 150 mM. In one embodiment, the replacement solution is sodium chloride at about 200 mM. In one embodiment, the replacement solution is sodium chloride at about 250 mM. In one embodiment, the replacement solution is sodium chloride at about 300 mM. In one embodiment, the replacement solution is sodium chloride at about 350 mM. In one embodiment, the replacement solution is sodium chloride at about 400 mM. In one embodiment, the replacement solution is sodium chloride at about 450 mM. In one embodiment, the replacement solution is sodium chloride at about 500 mM.

In an embodiment of the present invention, the number of diavolumes is at least 5, 10, 15, 20, 25, 30, 35, 40, 45, or 50. In an embodiment of the present invention, the number of diavolumes is between 1 and 50. In an embodiment of the present invention, the number of diavolumes is between 5 and 30. In an embodiment of the present invention, the number of diavolumes is between 5 and 20. In an embodiment of the present invention, the number of diavolumes is between 5 and 10. In an embodiment of the present invention, the number of diavolumes is about 1. In an embodiment of the present invention, the number of diavolumes is about 2. In an embodiment of the present invention, the number of diavolumes is about 3. In an embodiment of the present invention, the number of diavolumes is about 4. In an embodiment of the present invention, the number of diavolumes is about 5. In an embodiment of the present invention, the number of diavolumes is about 6. In an embodiment of the present invention, the number of diavolumes is about 7. In an embodiment of the present invention, the number of diavolumes is about 8. In an embodiment of the present invention, the number of diavolumes is about 9. In an embodiment of the present invention, the number of diavolumes is about 10. In an embodiment of the present invention, the number of diavolumes is about 11. In an embodiment of the present invention, the number of diavolumes is about 12. In an embodiment of the present invention, the number of diavolumes is about 13. In an embodiment of the present invention, the number of diavolumes is about 14. In an embodiment of the present invention, the number of diavolumes is about 15. In an embodiment of the present invention, the number of diavolumes is about 20. In an embodiment of the present invention, the number of diavolumes is about 25. In an embodiment of the present invention, the number of diavolumes is about 30. In an embodiment of the present invention, the number of diavolumes is about 35. In an embodiment of the present invention, the number of diavolumes is about 40. In an embodiment of the present invention, the number of diavolumes is about 45. In an embodiment of the present invention, the number of diavolumes is about 50. In an embodiment of the present invention, the number of diavolumes is about 60. In an embodiment of the present invention, the number of diavolumes is about 70. In an embodiment of the present invention, the number of diavolumes is about 80. In an embodiment of the present invention, the number of diavolumes is about 90. In an embodiment of the present invention, the number of diavolumes is about 100.

1.6 Flocculation

The methods of the invention may comprise a flocculation step. The inventors have found that the process results in a purified polysaccharide with low contamination. The inventor's process can be quick and simple.

Therefore, in an embodiment of the present invention, the solution obtained after base treatment by any of the method of section 1.2 above is treated by flocculation. In an embodiment the polysaccharide-containing solution is then collected for storage and/or additional processing.

In another embodiment of the present invention, the solution treated by the solid/liquid separation step of section 1.3 above is further treated by flocculation. In an embodiment, the solution treated by the solid/liquid separation step of section 1.3 is further filtrated as disclosed at section 1.4 above and is then further treated by flocculation. In an embodiment, the solution treated by the solid/liquid separation step of section 1.3 is further filtrated as disclosed at section 1.4 and further clarified by Ultrafiltration and/or Diafiltration as disclosed at section 1.5 above and is then further treated by flocculation

In the present invention, the term “flocculation” refers to a process wherein colloids come out of suspension in the form of floc or flake due to the addition of a flocculating agent.

The flocculation step comprises adding a “flocculating agent” to a solution comprising bacterial polysaccharides together with contaminants. In an embodiment, the contaminants comprise bacterial cell debris, bacterial cell proteins and nucleic acids. In an embodiment, the contaminants comprise bacterial cell proteins and nucleic acids.

As it will be further disclosed herebelow, the flocculation step may further include adjustment of the pH, either before or after the addition of the flocculating agent. In particular the solution may be acidified.

Furthermore, the addition of the flocculating agent and/or the adjustment of the pH and/or the settling may be performed at a temperature adjusted to a desirable level

Therefore in an embodiment the flocculation step comprises adding a flocculating agent to the solution, adjustment of the pH and adjustment of the temperature.

These steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be hold for some time to allow settling of the flocs prior to downstream processing.

In the present invention a “flocculating agent” refers to an agent being capable of allowing, in a solution comprising a polysaccharide of interest together with contaminants, promoting flocculation by causing colloids and other suspended particles to aggregate in the form of floc or flake, while the polysaccharide of interest significantly stays in solution.

In an embodiment of the present invention, the flocculating agent comprises a multivalent cation. In an embodiment, the flocculating agent is a multivalent cation. In a preferred embodiment said multivalent cation is selected from the group consisting of aluminium, iron, calcium and magnesium. In a preferred embodiment said multivalent cation comprises at least one of aluminium, iron, calcium or magnesium. In an embodiment the flocculating agent is a mixture of at least two multivalent cations selected from the group consisting of aluminium, iron, calcium and magnesium. In an embodiment the flocculating agent is a mixture of at least three multivalent cations selected from the group consisting of aluminium, iron, calcium and magnesium. In an embodiment the flocculating agent is a mixture of four multivalent cations consisting of aluminium, iron, calcium and magnesium.

In an embodiment, the flocculating agent comprises an agent selected from the group consisting of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent comprises at least one of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate or sodium silicate. In an embodiment, the flocculating agent is selected from the group consisting of alum (e.g. potassium alum, sodium alum or ammonium alum), aluminium chlorohydrate, aluminium sulphate, magnesium chloride, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent comprises at least one of alum (e.g. potassium alum, sodium alum or ammonium alum), aluminium chlorohydrate, aluminium sulphate, magnesium chloride, calcium oxide, calcium hydroxide, iron(II) sulphate (ferrous sulphate), iron(III) chloride (ferric chloride), polyacrylamide, modified polyacrylamides, polyDADMAC, sodium aluminate or sodium silicate. In an embodiment, the flocculating agent is aluminium sulphate. In an embodiment, the flocculating agent is calcium oxide. In an embodiment, the flocculating agent is calcium hydroxide. In an embodiment, the flocculating agent is iron(II) sulphate (ferrous sulphate). In an embodiment, the flocculating agent is iron(III) chloride (ferric chloride). In an embodiment, the flocculating agent is polyacrylamide. In an embodiment, the flocculating agent is polyDADMAC. In an embodiment, the flocculating agent is sodium aluminate. In an embodiment, the flocculating agent is sodium silicate. In an embodiment, the flocculating agent is aluminium chlorohydrate. In an embodiment, the flocculating agent is polyethylenimine (PEI). In an embodiment, the flocculating agent comprises alum. In an embodiment, the flocculating agent comprises magnesium chloride. In an embodiment, the flocculating agent is alum. In an embodiment, the flocculating agent is magnesium chloride. In an embodiment, the flocculating agent comprises potassium alum. In an embodiment, the flocculating agent is potassium alum. In an embodiment, the flocculating agent comprises sodium alum. In an embodiment, the flocculating agent is sodium alum. In an embodiment, the flocculating agent comprises ammonium alum. In an embodiment, the flocculating agent is ammonium alum.

In an embodiment, the flocculating agent is a mixture of agents (e.g. two, three or four agents) selected from the group consisting of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate. In an embodiment, the flocculating agent is selected from the group consisting of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate or sodium silicate.

In an embodiment, the flocculating agent is a mixture of two agents selected from the group consisting of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate. In an embodiment, the flocculating agent is a mixture of at least three agents selected from the group consisting of magnesium chloride, alum (e.g. potassium alum, sodium alum or ammonium alum), aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

In an embodiment, the flocculating agent comprises an agent selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent comprises at least one of chitosan, isinglass, moringa oleifera seeds (Horseradish Tree), gelatin, strychnos potatorum seeds (Nirmali nut tree), guar gum or alginates (e.g. brown seaweed extracts). In an embodiment, the flocculating agent is selected from the group consisting of chitosan, isinglass, moringa oleifera seeds (Horseradish Tree), gelatin, strychnos potatorum seeds (Nirmali nut tree), guar gum and alginates (e.g. brown seaweed extracts). In an embodiment, the flocculating agent comprises at least one of chitosan, isinglass, moringa oleifera seeds (Horseradish Tree), gelatin, strychnos potatorum seeds (Nirmali nut tree), guar gum or alginates (e.g. brown seaweed extracts).

The concentration of flocculating agent may depend on the agent(s) used, the polysaccharide of interest and the parameter of the flocculation step (e.g. temperature etc. . . . ).

In embodiments where the flocculating agent comprises magnesium chloride, a concentration of flocculating agent of between about 5 and 500 mM can be used. In embodiments where the flocculating agent is magnesium chloride, a concentration of flocculating agent of between about 5 and 500 mM can be used.

Preferably through a concentration of flocculating agent of between about 10 and 200 mM is used. Even more preferably a concentration of flocculating agent of between about 15 and 150 mM is used.

Any number within any of the above ranges is contemplated as an embodiment of the disclosure.

In an embodiment, a concentration of flocculating agent of about 5 mM is used. In an embodiment, a concentration of flocculating agent of about 7 mM is used. In an embodiment, a concentration of flocculating agent of about 10 mM is used. In an embodiment, a concentration of flocculating agent of about 15 mM is used. In an embodiment, a concentration of flocculating agent of about 20 mM is used. In an embodiment, a concentration of flocculating agent of about 30 mM is used. In an embodiment, a concentration of flocculating agent of about 50 mM is used. In an embodiment, a concentration of flocculating agent of about 75 mM is used. In an embodiment, a concentration of flocculating agent of about 100 mM is used. In an embodiment, a concentration of flocculating agent of about 150 mM is used. In an embodiment, a concentration of flocculating agent of about 200 mM is used. In an embodiment, a concentration of flocculating agent of about 250 mM is used. In an embodiment, a concentration of flocculating agent of about 300 mM is used. In an embodiment, a concentration of flocculating agent of about 400 mM is used. In an embodiment, a concentration of flocculating agent of about 450 mM is used. In an embodiment, a concentration of flocculating agent of about 500 mM is used.

In some embodiments of the present invention, the flocculating agent is added over a certain period of time. In some embodiments of the present invention, the flocculating agent is added over a period of between a few seconds (e.g. 1 to 10 seconds) and about one month. In some embodiments the flocculating agent is added over a period of between about 2 seconds and about two weeks. In some embodiments of the present invention, the flocculating agent is added over a period of between about 1 minute and about one week. In some embodiments the flocculating agent is added over a period of between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore, in certain embodiments, the flocculating agent is added over a period of between about 5 minutes and about one day. In certain embodiments, the flocculating agent is added over a period of between about 10 minutes and about one day. In certain embodiments, the flocculating agent is added over a period of between about 20 minutes and about one day. In certain embodiments, the flocculating agent is added over a period of between about 30 minutes and about one day. In certain embodiments, the flocculating agent is added over a period of between about 60 minutes and about one day. In certain embodiments, the flocculating agent is added over a period of between about 120 minutes and about one day. In certain embodiments, the flocculating agent is added over a period of between about 180 minutes and about one day. In certain embodiments, the flocculating agent is added over a period of between about 5 hours and about one day. In certain embodiments, the flocculating agent is added over a period of between about 10 hours and about one day. In certain embodiments, the flocculating agent is added over a period of between about 12 hours and about one day.

Preferably the flocculating agent is added over a period of between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In certain embodiments the flocculating agent is added over a period of between about 15 minutes and about 3 hours. In certain embodiments the flocculating agent is added over a period of between about 30 minutes and about 120 minutes.

Any number within any of the above ranges is contemplated as an embodiment of the disclosure.

The flocculating agent may be added over a period of about 2 seconds. The flocculating agent may be added over a period of about 10 seconds. The flocculating agent may be added over a period of about 30 seconds. The flocculating agent may be added over a period of about 1 minute. The flocculating agent may be added over a period of about 5 minutes. The flocculating agent may be added over a period of about 10 minutes. The flocculating agent may be added over a period of about 20 minutes. The flocculating agent may be added over a period of about 30 minutes. The flocculating agent may be added over a period of about 60 minutes. The flocculating agent may be added over a period of about 90 minutes. The flocculating agent may be added over a period of about 120 minutes. The flocculating agent may be added over a period of about 3 hours. The flocculating agent may be added over a period of about 6 hours. The flocculating agent may be added over a period of about 12 hours. The flocculating agent may be added over a period of about 24 hours. The flocculating agent may be added over a period of about 48 hours. The flocculating agent may be added over a period of about 3 days. The flocculating agent may be added over a period of about 7 days. The flocculating agent may be added over a period of about 14 days.

In an embodiment, the flocculating agent is added without agitation. In another embodiment, the flocculating agent is added under agitation. In another embodiment, the flocculating agent is added under gentle agitation. In another embodiment, the flocculating agent is added under vigorous agitation.

The inventors have further surprisingly noted that the flocculation is improved when performed at an acidic pH.

Therefore, in an embodiment of the present invention, the flocculation step is performed at a pH below 7.0. In a particular embodiment of the present invention, the flocculation step is performed at a pH below 6.0. In a particular embodiment of the present invention, the flocculation step is performed at a pH below 5.0. In a particular embodiment of the present invention, the flocculation step is performed at a pH below 4.0. In a particular embodiment of the present invention, the flocculation step is performed at a pH between 7.0 and 1.0. In an embodiment, the flocculation step is performed at a pH between 5.5 and 2.5. In an embodiment, the flocculation step is performed at a pH between 5.0 and 2.5. In an embodiment, the flocculation step is performed at a pH between 4.5 and 2.5. In an embodiment, the flocculation step is performed at a pH between 4.0 and 2.5. In an embodiment, the flocculation step is performed at a pH between 5.5 and 3.0. In an embodiment, the flocculation step is performed at a pH between 5.0 and 3.0. In an embodiment, the flocculation step is performed at a pH between 4.5 and 3.0. In an embodiment, the flocculation step is performed at a pH between 4.0 and 3.0. In an embodiment, the flocculation step is performed at a pH between 5.5 and 3.5. In an embodiment, the flocculation step is performed at a pH between 5.0 and 3.5. In an embodiment, the flocculation step is performed at a pH between 4.5 and 3.5. In an embodiment, the flocculation step is performed at a pH between 4.0 and 3.5. In an embodiment, the flocculation step is performed at a pH of about 5.5. In an embodiment, the flocculation step is performed at a pH of about 5.0. In an embodiment, the flocculation step is performed at a pH of about 4.5. In an embodiment, the flocculation step is performed at a pH of about 4.0. In an embodiment, the flocculation step is performed at a pH of about 3.5. In an embodiment, the flocculation step is performed at a pH of about 3.0. In an embodiment, the flocculation step is performed at a pH of about 2.5. In an embodiment, the flocculation step is performed at a pH of about 2.0. In an embodiment, the flocculation step is performed at a pH of about 1.5. In an embodiment, the flocculation step is performed at a pH of about 1.0. In an embodiment, the flocculation step is performed at a pH of about 4.0, about 3.5, about 3.0 or about 2.5. In an embodiment, the flocculation step is performed at a pH of about 3.5.

Any number within any of the above ranges is contemplated as an embodiment of the disclosure.

In an embodiment, said acidic pH is obtained by acidifying the solution with an acid. In an embodiment said acid is selected from the group consisting of HCl, H₃PO₄, citric acid, acetic acid, nitrous acid, and sulfuric acid. In an embodiment said acid is HCl, H₃PO₄, citric acid, acetic acid, nitrous acid or sulfuric acid. In an embodiment said acid is an amino acid. In an embodiment said acid is an amino acid selected from the group consisting of glycine, alanine and glutamate. In an embodiment said acid is glycine, alanine or glutamate. In an embodiment said acid is HCl (hydrochloric acid). In an embodiment said acid is sulfuric acid.

In an embodiment, the acid is added is without agitation. Preferably, the acid is added is under agitation. In an embodiment, the acid is added under gentle agitation. In an embodiment, the acid is added under vigorous agitation.

In some embodiments of the present invention, following the addition of the flocculating agent (and the optional acidification), the solution is hold for some time to allow settling of the flocs prior to downstream processing.

In some embodiments of the present invention, the flocculation step is performed with a settling time of between a few seconds (e.g. 2 to 10 seconds) to about 1 minute.

Preferably the settling time is at least about 2, at least about 3, at least about 4, at least about 5, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 115, at least about 120, at least about 125, at least about 130, at least about 135, at least about 140, at least about 145, at least about 150, at least about 155 or at least about 160 minutes. Preferably the settling time is less than a week. However, the settling time maybe longer.

Therefore in certain embodiments, the settling time is between about 1, about 2, about 3, about 4, about 5, about 6, about 7, about 8, about 9, about 10, about 15, about 20, about 25, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380, about 1440 minute(s), about two days, about three days, about four days, about five days or about six days and 1 week. In some embodiments of the present invention, the settling time is between a few seconds (e.g. 1 to 10 seconds) and about one month. In some embodiments the settling time is between about 2 seconds and about two weeks. In some embodiments of the present invention, the settling time is between about 1 minute and about one week. In some embodiments the settling time is between about 5 minutes and about two weeks. In some embodiments the settling time is between about 10 minutes and about two weeks. In some embodiments the settling time is between about 15 minutes and about two weeks. In some embodiments the settling time is between about 30 minutes and about two weeks. In some embodiments the settling time is between about 45 minutes and about two weeks. In some embodiments the settling time is between about 1 hour and about two weeks. In some embodiments the settling time is between about 2 hours and about two weeks. In some embodiments the settling time is between about 3 hours and about two weeks. In some embodiments the settling time is between about 4 hours and about two weeks. In some embodiments the settling time is between about 6 hours and about two weeks. In some embodiments the settling time is between about 12 hours and about two weeks. In some embodiments the settling time is between about 24 hours and about two weeks. In some embodiments the settling time is between about 36 hours and about two weeks. In some embodiments the settling time is between about 48 hours and about two weeks. In some embodiments the settling time is between about 72 hours and about two weeks. In some embodiments the settling time is between about 96 hours and about two weeks.

In some embodiments the settling time is between about 1 minute, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours and about two days.

Therefore in certain embodiments, the settling time is between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

Preferably the settling time is between about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 80 minutes, about 85 minutes, about 90 minutes, about 95 minutes, about 100 minutes, about 110 minutes, about 120 minutes, about 130 minutes, about 140 minutes, about 150 minutes, about 160 minutes, about 170 minutes, about 3 hours, about 4 hours, about 5 hours, about 6 hours, about 7 hours, about 8 hours, about 9 hours, about 10 hours, about 11 hours or about 12 hours and about one day.

In some embodiments the settling time is between about 2 seconds and about one day. In some embodiments of the present invention, the settling time is between about 1 minute and about one day. In some embodiments the settling time is between about 5 minutes and about one day. In some embodiments the settling time is between about 10 minutes and about one day. In some embodiments the settling time is between about 15 minutes and about one day. In some embodiments the settling time is between about 30 minutes and about one day. In some embodiments the settling time is between about 45 minutes and about one day. In some embodiments the settling time is between about 1 hour and about one day. In some embodiments the settling time is between about 2 hours and about one day. In some embodiments the settling time is between about 3 hours and about one day. In some embodiments the settling time is between about 4 hours and about one day. In some embodiments the settling time is between about 6 hours and about one day. In some embodiments the settling time is between about 12 hours and about one day.

In some embodiments the settling time is between about 2 seconds and about 12 hours. In some embodiments of the present invention, the settling time is between about 1 minute and about 12 hours. In some embodiments the settling time is between about 5 minutes and about 12 hours. In some embodiments the settling time is between about 10 minutes and about 12 hours. In some embodiments the settling time is between about 15 minutes and about 12 hours. In some embodiments the settling time is between about 30 minutes and about 12 hours. In some embodiments the settling time is between about 45 minutes and about 12 hours. In some embodiments the settling time is between about 1 hour and about 12 hours. In some embodiments the settling time is between about 2 hours and about 12 hours. In some embodiments the settling time is between about 3 hours and about 12 hours. In some embodiments the settling time is between about 4 hours and about 12 hours. In some embodiments the settling time is between about 6 hours and about 12 hours.

In certain embodiments the settling time is between about 15 minutes and about 3 hours. In certain embodiments the settling time is between about 30 minutes and about 120 minutes.

Any number within any of the above ranges is contemplated as an embodiment of the disclosure.

In certain embodiments the settling time is about 2 seconds. In certain embodiments the settling time is about 10 seconds. In certain embodiments the settling time is about 30 seconds. In certain embodiments the settling time is about 1 minute. In certain embodiments the settling time is about 5 minutes. In certain embodiments the settling time is about 10 minutes. In certain embodiments the settling time is about 15 minutes. In certain embodiments the settling time is about 20 minutes. In certain embodiments the settling time is about 25 minutes. In certain embodiments the settling time is about 30 minutes. In certain embodiments the settling time is about 45 minutes. In certain embodiments the settling time is about 60 minutes. In certain embodiments the settling time is about 90 minutes. In certain embodiments the settling time is about 120 minutes. In certain embodiments the settling time is about 3 hours. In certain embodiments the settling time is about 4 hours. In certain embodiments the settling time is about 6 hours. In certain embodiments the settling time is about 8 hours. In certain embodiments the settling time is about 12 hours. In certain embodiments the settling time is about 18 hours. In certain embodiments the settling time is about 24 hours. In certain embodiments the settling time is about 30 hours. In certain embodiments the settling time is 48 hours. In certain embodiments the settling time is about 3 days. In certain embodiments the settling time is about 5 days. In certain embodiments the settling time is about 7 days. In certain embodiments the settling time is about 10 days. In certain embodiments the settling time is about 15 days.

Preferably the settling time is between about 5, about 10, about 15, about 20, about 25, about 30, about 60, about 90, about 120, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 or about 1440 minute(s) and two days. In certain embodiments the settling time is between about 5 minutes and about one day. In certain embodiments the settling time is between about 5 minutes and about 120 minutes.

Any number within any of the above ranges is contemplated as an embodiment of the disclosure.

In an embodiment, the optional settling step is conducted without agitation. In an embodiment, the optional settling step is conducted under agitation. In another embodiment, the optional settling step is conducted under gentle agitation. In another embodiment, the optional settling step is conducted under vigorous agitation.

In an embodiment of the present invention, the addition of the flocculating agent, the settling of the solution and/or the adjustment of the pH is performed at a temperature between about 4° C. and about 30° C. In an embodiment, the addition of the flocculating agent, the settling of the solution and/or the adjustment of the pH is performed at a temperature of about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C. or about 30° C. In an embodiment, the addition of the flocculating agent, the settling of the solution and/or the adjustment of the pH is performed at a temperature of about 20° C.

In an embodiment, the addition of the flocculating agent, the settling of the solution and/or the adjustment of the pH is performed at a temperature of about 15° C. In an embodiment, the addition of the flocculating agent, the settling of the solution and/or the adjustment of the pH is performed at a temperature of about 25° C.

The inventors have surprisingly noted that the flocculation can be further improved when performed at elevated temperature. Therefore, in a particular embodiment of the present invention, the addition of the flocculating agent, the settling of the solution and/or the adjustment of the pH is performed at temperature between about 30° C. to about 95° C. In an embodiment, the addition of the flocculating agent, the settling of the solution and/or the adjustment of the pH is performed at a temperature between about 35° C. to about 80° C. In an embodiment, the addition of the flocculating agent, the settling of the solution and/or the adjustment of the pH is performed at a temperature at temperature between about 40° C. to about 70° C. In an embodiment, the addition of the flocculating agent, the settling of the solution and/or the adjustment of the pH is performed at temperature between about 45° C. to about 65° C. In an embodiment, the addition of the flocculating agent, the settling of the solution and/or the adjustment of the pH is performed at temperature between about 50° C. to about 60° C. In an embodiment, the addition of the flocculating agent, the settling of the solution and/or the adjustment of the pH is performed at temperature between about 50° C. to about 55° C. In an embodiment, the addition of the flocculating agent, the settling of the solution and/or the adjustment of the pH is performed at temperature between about 45° C. to about 55° C. In an embodiment, the addition of the flocculating agent, the settling of the solution and/or the adjustment of the pH is performed at temperature between about 45° C. to about 55° C. In an embodiment, the addition of the flocculating agent, the settling of the solution and/or the adjustment of the pH is performed at a temperature of about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C. or about 80° C. In an embodiment, the addition of the flocculating agent, the settling of the solution and/or the adjustment of the pH is performed at a temperature of about 50° C.

Any number within any of the above ranges is contemplated as an embodiment of the disclosure.

In an embodiment, the addition of the flocculating agent is performed at any of the above mentioned temperatures.

In an embodiment, the settling of the solution after the addition of the flocculating agent is performed at any of the above mentioned temperatures.

In an embodiment, the adjustment of the pH is performed at any of the above mentioned temperatures.

In an embodiment, the addition of the flocculating agent and the settling of the solution after the addition of the flocculating agent are performed at any of the above mentioned temperatures.

In an embodiment, the addition of the flocculating agent and the adjustment of the pH are performed at any of the above mentioned temperatures.

In an embodiment, the addition of the flocculating, the settling of the solution after the addition of the flocculating agent and the adjustment of the pH are performed at any of the above mentioned temperatures.

In an embodiment, the flocculation step comprises adding a flocculating agent (as disclosed above) without pH adjustment.

In an embodiment, the flocculation step comprises adding a flocculating agent and settling the solution (as disclosed above), without pH adjustment.

In an embodiment, the flocculation step comprises adding a flocculating agent, adjusting the pH and settling the solution (as disclosed above). In an embodiment, the flocculating agent is added before adjusting the pH. In another embodiment, the pH is adjusted before adding the flocculating agent.

In an embodiment, the flocculation step comprises adding a flocculating agent, settling the solution and adjusting the pH (as disclosed above). In an embodiment, the addition of flocculating agent and settling of the solution is conducted before adjusting the pH. In another embodiment, the pH is adjusted before adding the flocculating agent and settling the solution. In an embodiment, the addition of the flocculating agent and adjusting the pH is conducted before settling the solution. In another embodiment, the pH is adjusted before adding the flocculating agent and settling the solution.

In an embodiment, the flocculation step comprises adding a flocculating agent, adjusting the pH and adjustment of the temperature (as disclosed above).

These steps can be performed in any order:
addition of the flocculating agent followed by adjustment of the pH followed by adjustment of the temperature or;
addition of the flocculating agent followed by adjustment of the temperature followed by adjustment of the pH or;
adjustment of the pH followed by addition of the flocculating agent followed by adjustment of the temperature or;
adjustment of the pH followed by adjustment of the temperature followed by addition of the flocculating agent or;
adjustment of the temperature followed by addition of the flocculating agent followed by adjustment of the pH or;
adjustment of the temperature followed by adjustment of the pH followed by addition of the flocculating agent.

Furthermore, following the addition of the flocculating agent and/or the adjustment of the pH, the solution may be hold for some time to allow settling of the flocs prior to downstream processing.

1.7 Solid/Liquid Separation

The flocculated material can be separated from the polysaccharide of interest by any suitable solid/liquid separation method.

Therefore, in an embodiment of the present invention, after flocculation, the suspension (as obtained at section 1.6 above) is clarified by decantation, sedimentation, filtration or centrifugation. In an embodiment the polysaccharide-containing solution is then collected for storage and/or additional processing.

In an embodiment of the present invention, after flocculation, the suspension (as obtained at section 1.6 above) is clarified by decantation. Decanters are used to separate liquids where there is a sufficient difference in density between the liquids for the floc to settle. In an operating decanter there will be three distinct zones: clear heavy liquid, separating dispersed liquid (the dispersion zone), and clear light liquid. To produce a clean solution, a small amount of solution must generally be left in the container. Decanters can be designed for continuous operation.

In an embodiment of the present invention, after flocculation, the suspension (as obtained at section 1.6 above) is clarified by sedimentation (settling). Sedimentation is the separation of suspended solid particles from a liquid mixture by gravity settling into a clear fluid and a slurry of higher solids content. Sedimentation can be done in a thickener, in a clarifier or in a classifier. Since thickening and clarification are relatively cheap processes when used for the treatment of large volumes of liquid, they can be used for pre-concentration of feeds to filtering.

In an embodiment of the present invention, after flocculation, the suspension (as obtained at section 1.6 above) is clarified by centrifugation. In an embodiment said centrifugation is continuous centrifugation. In an embodiment said centrifugation is bucket centrifugation. In an embodiment the polysaccharide-containing supernatant is then collected for storage and/or additional processing.

In some embodiments the suspension is centrifuged at about 1,000 g about 2,000 g, about 3,000 g, about 4,000 g, about 5,000 g, about 6,000 g, about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g, about 25,000 g, about 30,000 g, about 35,000 g, about 40,000 g, about 50,000 g, about 60,000 g, about 70,000 g, about 80,000 g, about 90,000 g, about 100,000 g, about 120,000 g, about 140,000 g, about 160,000 g or about 180,000 g. In some embodiments the suspension is centrifuged at about 8,000 g, about 9,000 g, about 10,000 g, about 11,000 g, about 12,000 g, about 13,000 g, about 14,000 g, about 15,000 g, about 16,000 g, about 17,000 g, about 18,000 g, about 19,000 g, about 20,000 g or about 25,000 g. In some embodiments the suspension is centrifuged between about 5,000 g and about 25,000 g. In some embodiments the suspension is centrifuged between about 8,000 g and about 20,000 g. In some embodiments the suspension is centrifuged between about 10,000 g and about 15,000 g. In some embodiments the suspension is centrifuged between about 10,000 g and about 12,000 g.

Any number within any of the above ranges is contemplated as an embodiment of the disclosure.

In some embodiments the suspension is centrifuged during at least 2, at least 3, at least 4, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155 or at least 160 minutes. Preferably the centrifugation time is less than 24 hours.

Therefore in certain embodiments, the suspension is centrifuged during between about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320 or about 1380 minutes and 1440 minutes.

Preferably the suspension is centrifuged during between about 5, about 10, about 15, about 20, about 25, about 30, about 60, about 90, about 120, about 180, about 240, about 300, about 360, about 420, about 480 or about 540 minutes and about 600 minutes. In certain embodiments the suspension is centrifuged during between about 5 minutes and about 3 hours. In certain the suspension is centrifuged during between about 5 minutes and about 120 minutes.

The suspension may be centrifuged during between about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes or about 155 minutes and about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes or about 55 minutes and about 60 minutes.

The suspension may be centrifuged during about 5, about 10, about 15, about 20, about 30, about 40, about 50, about 60, about 70, about 80, about 90, about 100, about 120, about 140, about 160, about 180, about 220, about 240, about 300, about 360, about 420, about 480, about 540, about 600, about 660, about 720, about 780, about 840, about 900, about 960, about 1020, about 1080, about 1140, about 1200, about 1260, about 1320, about 1380 minutes or about 1440 minutes.

The suspension may be centrifuged during about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, about 125 minutes, about 130 minutes, about 135 minutes, about 140 minutes, about 145 minutes, about 150 minutes, about 155 minutes or about 160 minutes.

The suspension may be centrifuged during between about 10 minutes, about 15 minutes, about 20 minutes, about 25 minutes, about 30 minutes, about 35 minutes, about 40 minutes, about 45 minutes, about 50 minutes, about 55 minutes or about 60 minutes.

The suspension may be centrifuged during about 5 minutes. The suspension may be centrifuged during about 10 minutes. The suspension may be centrifuged during about 15 minutes. The suspension may be centrifuged during about 30 minutes. The suspension may be centrifuged during about 60 minutes. The suspension may be centrifuged during about 90 minutes. The suspension may be centrifuged during about 120 minutes. The suspension may be centrifuged during about 160 minutes.

Any number within any of the above ranges is contemplated as an embodiment of the disclosure.

In an embodiment of the present invention, centrifugation is continuous centrifugation. In said embodiment, the feed rate can be of between of 50-5000 ml/min. In an embodiment, the feed rate can be of between of 100-4000 ml/min. In an embodiment, the feed rate can be of between of 150-3000 ml/min. In an embodiment, the feed rate can be of between of 200-2500 ml/min. In an embodiment, the feed rate can be of between of 250-2000 ml/min. In an embodiment, the feed rate can be of between of 300-1500 ml/min. In an embodiment, the feed rate can be of between of 300-1000 ml/min. In an embodiment, the feed rate can be of between of 200-1000 ml/min. In an embodiment, the feed rate can be of between of 200-1500 ml/min. In an embodiment, the feed rate can be of between of 400-1500 ml/min. In an embodiment, the feed rate can be of between of 500-1500 ml/min. In an embodiment, the feed rate can be of between of 500-1000 ml/min. In an embodiment, the feed rate can be of between of 500-2000 ml/min. In an embodiment, the feed rate can be of between of 500-2500 ml/min. In an embodiment, the feed rate can be of between of 1000-2500 ml/min.

In an embodiment, the feed rate can be of about 10 ml/min. In an embodiment, the feed rate can be of about 25 ml/min. In an embodiment, the feed rate can be of about 50 ml/min. In an embodiment, the feed rate can be of about 75 ml/min. In an embodiment, the feed rate can be of about 100 ml/min. In an embodiment, the feed rate can be of about 200 ml/min. In an embodiment, the feed rate can be of about 300 ml/min. In an embodiment, the feed rate can be of about 400 ml/min. In an embodiment, the feed rate can be of about 500 ml/min. In an embodiment, the feed rate can be of about 700 ml/min. In an embodiment, the feed rate can be of about 1000 ml/min. In an embodiment, the feed rate can be of about 1500 ml/min. In an embodiment, the feed rate can be of about 2000 ml/min. In an embodiment, the feed rate can be of about 2500 ml/min. In an embodiment, the feed rate can be of about 3000 ml/min. In an embodiment, the feed rate can be of about 3500 ml/min. In an embodiment, the feed rate can be of about 4000 ml/min. In an embodiment, the feed rate can be of about 5000 ml/min.

The solid/liquid separation methods described above can be used in a standalone format or in combination of two in any order, or in combination of three in any order.

1.8 Neutralization

Once the solution has been treated by the flocculation step of section 1.6 above and/or by the solid/liquid separation step of section 1.7 above, the acidic pH of the polysaccharide containing solution (e.g. the supernatant) is raised.

Therefore, in an embodiment of the present invention, the pH of the polysaccharide containing solution is adjusted to a pH above 5.0. In an embodiment of the present invention, the pH of the polysaccharide containing solution is adjusted to a pH above 6.0. In an embodiment of the present invention, the pH of the polysaccharide containing solution is adjusted to a pH above 7.0. In an embodiment of the present invention, the pH of the polysaccharide containing solution is adjusted to a pH above 8.0. In an embodiment of the present invention, the pH of the polysaccharide containing solution is adjusted to a pH above 9.0. In a particular embodiment of the present invention, the solution is adjusted to a pH between 5.0 and 9.0. In a particular embodiment of the present invention, the solution is adjusted to a pH between 6.0 and 8.0. In a particular embodiment of the present invention, the solution is adjusted to a pH between 6.5 and 7.5. In an embodiment, the solution is adjusted to a pH of about 5.0. In an embodiment, the solution is adjusted to a pH of about 5.5. In an embodiment, the solution is adjusted to a pH of about 6.0. In an embodiment, the solution is adjusted to a pH of about 6.5. In an embodiment, the solution is adjusted to a pH of about 7.0. In an embodiment, the solution is adjusted to a pH of about 7.5. In an embodiment, the solution is adjusted to a pH of about 8.0. In an embodiment, the solution is adjusted to a pH of about 8.5. In an embodiment, the solution is adjusted to a pH of about 9.0. In an embodiment, the solution is adjusted to a pH of about 6.0, about 6.5, about 7.0, about 7.5 or about 8.0. In an embodiment, the solution is adjusted to a pH of about 7.0.

Any number within any of the above ranges is contemplated as an embodiment of the disclosure.

In an embodiment, said pH is raised by the addition of a base. In an embodiment, the base is selected from the group consisting of NaOH, KOH, LiOH, NaHCO₃, Na2C03, KzC03, KCN, Et3N, NH3, HzN2H2, NaH, NaOMe, NaOEt and KOtBu.

In an embodiment, the base comprises at least one of NaOH, KOH, LiOH, NaHCO₃, Na2C03, KzC03, KCN, Et3N, NH3, HzN2H2, NaH, NaOMe, NaOEt or KOtBu.

In an embodiment, the base is KOH. In an embodiment, the base is LiOH. In an embodiment, the base is NaHC03. In an embodiment, the base is Na2C03.

In an embodiment, the base is NaOH.

In an embodiment, the pH is raised by the addition of a base having a concentration of between 0.1N and 10N. In an embodiment, the pH is raised by the addition of a base having a concentration of between 0.5N and 5N. In an embodiment, the pH is raised by the addition of a base having a concentration of between 1N and 3N. In an embodiment, the pH is raised by the addition of a base having a concentration of between 1N and 2N. In an embodiment, the pH is raised by the addition of a base having a concentration selected from the group consisting of 0.1N, 0.5N, 1N, 2N, 3N, 4N, 5N, 6N, 7N, 8N, 9N and 10N. In an embodiment, the pH is raised by the addition of a base having a concentration of about 0.1N, about 0.5N, about 1N, about 2N, about 3N, about 4N, about 5N, about 6N, about 7N, about 8N, about 9N or about 10N.

In an embodiment, the base is added is without agitation. Preferably, the base is added under agitation. In an embodiment, the base is added under gentle agitation. In an embodiment, the base is added under vigorous agitation.

1.9 Activated Carbon Filtration

Once the solution has been treated by the flocculation step of section 1.6 above, the solution containing the polysaccharide can optionally be further clarified by an activated carbon filtration step.

In an embodiment, the solution of section 1.6 further treated by the solid/liquid separation of step of section 1.7 (e.g. the supernatant) is further clarified by an activated carbon filtration step. In an embodiment, the solution further neutralized by the neutralization step of section 1.8 above is further clarified by an activated carbon filtration step.

A step of activated carbon filtration allows for further removing host cell impurities such as proteins and nucleic acids as well as colored impurities (see WO2008/118752).

In an embodiment, activated carbon (also named active charcoal) is added to the solution in an amount sufficient to absorb the majority of the proteins and nucleic acids contaminants, and then removed once the contaminants have been adsorbed onto activated carbon. In an embodiment the activated carbon is added in the form of a powder, as a granular carbon bed, as a pressed carbon block or extruded carbon block (see e.g. Norit active charcoal). In an embodiment the activated carbon is added in the form of a powder. In an embodiment the activated carbon is added in the form of a granular carbon bed. In an embodiment the activated carbon is added in the form of a pressed carbon block or extruded carbon block. In an embodiment, the activated carbon is added in an amount of about 0.1 to 20% (weight volume). In an embodiment, the activated carbon is added in an amount of about 1 to 15% (weight volume). In an embodiment, the activated carbon is added in an amount of about 1 to 10% (weight volume). In an embodiment, the activated carbon is added in an amount of about 2 to 10% (weight volume). In an embodiment, the activated carbon is added in an amount of about 3 to 10% (weight volume). In an embodiment, the activated carbon is added in an amount of about 4 to 10% (weight volume). In an embodiment, the activated carbon is added in an amount of about 5 to 10% (weight volume). In an embodiment, the activated carbon is added in an amount of about 1 to 5% (weight volume). In an embodiment, the activated carbon is added in an amount of about 2 to 5% (weight volume). The mixture is then stirred and left to stand. In an embodiment, the mixture is left to stand for about 5, 10, 15, 20, 30, 45, 60, 90, 120, 180, 240 minutes or more. In an embodiment, the mixture is left to stand for about 5 minutes. In an embodiment, the mixture is left to stand for about 15 minutes. In an embodiment, the mixture is left to stand for about 30 minutes.

In an embodiment, the mixture is left to stand for about 45 minutes In an embodiment, the mixture is left to stand for about 60 minutes. In an embodiment, the mixture is left to stand for about 90 minutes. In an embodiment, the mixture is left to stand for about 120 minutes. In an embodiment, the mixture is left to stand for about 180 minutes. In an embodiment, the mixture is left to stand for about 240 minutes. In an embodiment, the mixture is left to stand for more than 240 minutes and less than one day. In an embodiment, the mixture is left to stand for more than 240 minutes and less than a week. The activated carbon is then removed. The activated carbon can be removed for example by centrifugation or filtration.

In a preferred embodiment, the solution is filtered through activated carbon immobilized in a matrix. The matrix may be any porous filter medium permeable for the solution. The matrix may comprise a support material and/or a binder material. The support material may be a synthetic polymer. The support material may be a polymer of natural origin. Suitable synthetic polymers may include polystyrene, polyacrylamide and polymethyl methacrylate. Polymers of natural origin may include cellulose, polysaccharide and dextran, agarose. Typically, the polymer support material is in the form of a fibre network to provide mechanical rigidity. The binder material may be a resin. The matrix may have the form of a membrane sheet. In an embodiment, the activated carbon immobilized in the matrix is in the form of a flow-through carbon cartridge. A cartridge is a self-contained entity containing powdered activated carbon immobilized in the matrix and prepared in the form of a membrane sheet. The membrane sheet may be captured in a plastic permeable support to form a disc.

Alternatively, the membrane sheet may be spirally wound. To increase filter surface area, several discs may be stacked upon each other. In particular, the discs stacked upon each other have a central core pipe for collecting and removing the carbon-treated sample from the filter. The configuration of stacked discs may be lenticular.

The activated carbon in the carbon filter may be derived from different raw materials, e.g. peat, lignite, wood or coconut shell.

Any process known in the art, such as steam or chemical treatment, may be used to activate carbon (e.g. wood-based phosphoric acid-activated carbon).

In the present invention, activated carbon immobilized in a matrix may be placed in a housing to form an independent filter unit. Each filter unit has its own in-let and out-let for the solution to be purified. Examples of filter units that are usable in the present invention are the carbon cartridges from Cuno Inc. (Meriden, USA) or Pall Corporation (East Hill, USA). In particular, CUNO zetacarbon filters are suitable for use in the invention. These carbon filters comprise a cellulose matrix into which activated carbon powder is entrapped and resin-bonded in place.

In an embodiment, the activated carbon filter disclosed above has a nominal micron rating of between about 0.01-100 micron. In an embodiment, the activated carbon filter disclosed above has a nominal micron rating of between about 0.05-100 micron. In an embodiment, the activated carbon filter disclosed above has a nominal micron rating of between about 0.1-100 micron. In an embodiment, the activated carbon filter disclosed above has a nominal micron rating of between about 0.2-100 micron. In an embodiment, the activated carbon filter disclosed above has a nominal micron rating of between about 0.3-100 micron. In an embodiment, the activated carbon filter disclosed above has a nominal micron rating of between about 0.4-100 micron. In an embodiment, the activated carbon filter disclosed above has a nominal micron rating of between about 0.5-100 micron. In an embodiment, the activated carbon filter disclosed above has a nominal micron rating of between about 0.6-100 micron, about 0.7-100 micron, about 0.8-100 micron. In an embodiment, the activated carbon filter disclosed above has a nominal micron rating of between about 0.9-100 micron. In an embodiment, the activated carbon filter disclosed above has a nominal micron rating of between about 1-100 micron. In an embodiment, the activated carbon filter disclosed above has a nominal micron rating of between about 1.25-100 micron. In an embodiment, the activated carbon filter disclosed above has a nominal micron rating of between about 1.5-100 micron. In an embodiment, the activated carbon filter disclosed above has a nominal micron rating of between about 1.75-100 micron. In an embodiment, the activated carbon filter disclosed above has a nominal micron rating of between about 2-100 micron. In an embodiment, the activated carbon filter disclosed above has a nominal micron rating of between about 3-100 micron. In an embodiment, the activated carbon filter disclosed above has a nominal micron rating of between In an embodiment, the activated carbon filter disclosed above has a nominal micron rating of between about 5-100 micron. In an embodiment, the activated carbon filter disclosed above has a nominal micron rating of between about 6-100 micron. In an embodiment, the activated carbon filter disclosed above has a nominal micron rating of between about 7-100 micron. In an embodiment, the activated carbon filter disclosed above has a nominal micron rating of between about 10-100 micron. In an embodiment, the activated carbon filter disclosed above has a nominal micron rating of between about 15-100 micron. In an embodiment, the activated carbon filter disclosed above has a nominal micron rating of between about 20-100 micron. In an embodiment, the activated carbon filter disclosed above has a nominal micron rating of between about 25-100 micron. In an embodiment, the activated carbon filter disclosed above has a nominal micron rating of between about 30-100 micron. In an embodiment, the activated carbon filter disclosed above has a nominal micron rating of between about 40-100 micron. In an embodiment, the activated carbon filter disclosed above has a nominal micron rating of between about 50-100 micron. In an embodiment, the activated carbon filter disclosed above has a nominal micron rating of between about 75-100 micron.

In an embodiment, the activated carbon filter disclosed above has a nominal micron rating of between about 0.01-50 micron. In an embodiment, the activated carbon filter disclosed above has a nominal micron rating of between about 0.05-50 micron. In an embodiment, the activated carbon filter disclosed above has a nominal micron rating of between about 0.1-50 micron. In an embodiment, the activated carbon filter disclosed above has a nominal micron rating of between about 0.2-50 micron. In an embodiment, the activated carbon filter disclosed above has a nominal micron rating of between about 0.3-50 micron. In an embodiment, the activated carbon filter disclosed above has a nominal micron rating of between about 0.4-50 micron. In an embodiment, the activated carbon filter disclosed above has a nominal micron rating of between about 0.5-50 micron. In an embodiment, the activated carbon filter disclosed above has a nominal micron rating of between about 0.6-50 micron, about 0.7-50 micron, about 0.8-50 micron. In an embodiment, the activated carbon filter disclosed above has a nominal micron rating of between about 0.9-50 micron. In an embodiment, the activated carbon filter disclosed above has a nominal micron rating of between about 1-50 micron. In an embodiment, the activated carbon filter disclosed above has a nominal micron rating of between about 1.25-50 micron. In an embodiment, the activated carbon filter disclosed above has a nominal micron rating of between about 1.5-50 micron. In an embodiment, the activated carbon filter disclosed above has a nominal micron rating of between about 1.75-50 micron. In an embodiment, the activated carbon filter disclosed above has a nominal micron rating of between about 2-50 micron. In an embodiment, the activated carbon filter disclosed above has a nominal micron rating of between about 3-50 micron. In an embodiment, the activated carbon filter disclosed above has a nominal micron rating of between about 5-50 micron. In an embodiment, the activated carbon filter disclosed above has a nominal micron rating of between about 6-50 micron. In an embodiment, the activated carbon filter disclosed above has a nominal micron rating of between about 7-50 micron. In an embodiment, the activated carbon filter disclosed above has a nominal micron rating of between about 10-50 micron. In an embodiment, the activated carbon filter disclosed above has a nominal micron rating of between about 15-50 micron. In an embodiment, the activated carbon filter disclosed above has a nominal micron rating of between about 20-50 micron. In an embodiment, the activated carbon filter disclosed above has a nominal micron rating of between about 25-50 micron. In an embodiment, the activated carbon filter disclosed above has a nominal micron rating of between about 30-50 micron. In an embodiment, the activated carbon filter disclosed above has a nominal micron rating of between about 40-50 micron. In an embodiment, the activated carbon filter disclosed above has a nominal micron rating of between about 0.01-25 micron. In an embodiment, the activated carbon filter disclosed above has a nominal micron rating of between about 0.05-25 micron. In an embodiment, the activated carbon filter disclosed above has a nominal micron rating of between about 0.1-25 micron. In an embodiment, the activated carbon filter disclosed above has a nominal micron rating of between about 0.2-25 micron. In an embodiment, the activated carbon filter disclosed above has a nominal micron rating of between about 0.3-25 micron. In an embodiment, the activated carbon filter disclosed above has a nominal micron rating of between about 0.4-25 micron. In an embodiment, the activated carbon filter disclosed above has a nominal micron rating of between about 0.5-25 micron. In an embodiment, the activated carbon filter disclosed above has a nominal micron rating of between about 0.6-25 micron, about 0.7-25 micron, about 0.8-25 micron. In an embodiment, the activated carbon filter disclosed above has a nominal micron rating of between about 0.9-25 micron. In an embodiment, the activated carbon filter disclosed above has a nominal micron rating of between about 1-25 micron. In an embodiment, the activated carbon filter disclosed above has a nominal micron rating of between about 1.25-25 micron. In an embodiment, the activated carbon filter disclosed above has a nominal micron rating of between about 1.5-25 micron. In an embodiment, the activated carbon filter disclosed above has a nominal micron rating of between about 1.75-25 micron. In an embodiment, the activated carbon filter disclosed above has a nominal micron rating of between about 2-25 micron. In an embodiment, the activated carbon filter disclosed above has a nominal micron rating of between about 3-25 micron. In an embodiment, the activated carbon filter disclosed above has a nominal micron rating of between about 5-25 micron. In an embodiment, the activated carbon filter disclosed above has a nominal micron rating of between about 6-micron. In an embodiment, the activated carbon filter disclosed above has a nominal micron rating of between about 7-25 micron. In an embodiment, the activated carbon filter disclosed above has a nominal micron rating of between about 10-25 micron. In an embodiment, the activated carbon filter disclosed above has a nominal micron rating of between about 15-25 micron. In an embodiment, the activated carbon filter disclosed above has a nominal micron rating of between about 20-25 micron.

In an embodiment, the activated carbon filter disclosed above has a nominal micron rating of between about 0.01-10 micron. In an embodiment, the activated carbon filter disclosed above has a nominal micron rating of between about 0.05-10 micron. In an embodiment, the activated carbon filter disclosed above has a nominal micron rating of between about 0.1-10 micron. In an embodiment, the activated carbon filter disclosed above has a nominal micron rating of between about 0.2-10 micron. In an embodiment, the activated carbon filter disclosed above has a nominal micron rating of between about 0.3-10 micron. In an embodiment, the activated carbon filter disclosed above has a nominal micron rating of between about 0.4-10 micron. In an embodiment, the activated carbon filter disclosed above has a nominal micron rating of between about 0.5-10 micron. In an embodiment, the activated carbon filter disclosed above has a nominal micron rating of between about 0.6-10 micron, about 0.7-10 micron, about 0.8-10 micron. In an embodiment, the activated carbon filter disclosed above has a nominal micron rating of between about 0.9-10 micron. In an embodiment, the activated carbon filter disclosed above has a nominal micron rating of between about 1-10 micron. In an embodiment, the activated carbon filter disclosed above has a nominal micron rating of between about 1.25-10 micron. In an embodiment, the activated carbon filter disclosed above has a nominal micron rating of between about 1.5-10 micron. In an embodiment, the activated carbon filter disclosed above has a nominal micron rating of between about 1.75-10 micron. In an embodiment, the activated carbon filter disclosed above has a nominal micron rating of between about 2-10 micron. In an embodiment, the activated carbon filter disclosed above has a nominal micron rating of between about 3-10 micron. In an embodiment, the activated carbon filter disclosed above has a nominal micron rating of between about 5-10 micron. In an embodiment, the activated carbon filter disclosed above has a nominal micron rating of between about 6-micron. In an embodiment, the activated carbon filter disclosed above has a nominal micron rating of between about 7-10 micron.

In an embodiment, the activated carbon filter disclosed above has a nominal micron rating of between about 0.01-5 micron. In an embodiment, the activated carbon filter disclosed above has a nominal micron rating of between about 0.05-5 micron. In an embodiment, the activated carbon filter disclosed above has a nominal micron rating of between about 0.1-5 micron. In an embodiment, the activated carbon filter disclosed above has a nominal micron rating of between about 0.2-5 micron. In an embodiment, the activated carbon filter disclosed above has a nominal micron rating of between about 0.3-5 micron. In an embodiment, the activated carbon filter disclosed above has a nominal micron rating of between about 0.4-5 micron. In an embodiment, the activated carbon filter disclosed above has a nominal micron rating of between about 0.5-5 micron. In an embodiment, the activated carbon filter disclosed above has a nominal micron rating of between about 0.6-5 micron, about 0.7-5 micron, about 0.8-5 micron. In an embodiment, the activated carbon filter disclosed above has a nominal micron rating of between about 0.9-5 micron. In an embodiment, the activated carbon filter disclosed above has a nominal micron rating of between about 1-5 micron. In an embodiment, the activated carbon filter disclosed above has a nominal micron rating of between about 1.25-5 micron. In an embodiment, the activated carbon filter disclosed above has a nominal micron rating of between about 1.5-5 micron. In an embodiment, the activated carbon filter disclosed above has a nominal micron rating of between about 1.75-5 micron. In an embodiment, the activated carbon filter disclosed above has a nominal micron rating of between about 2-5 micron. In an embodiment, the activated carbon filter disclosed above has a nominal micron rating of between about 3-5 micron.

In an embodiment, the activated carbon filter disclosed above has a nominal micron rating of between about 0.01-2 micron. In an embodiment, the activated carbon filter disclosed above has a nominal micron rating of between about 0.05-2 micron. In an embodiment, the activated carbon filter disclosed above has a nominal micron rating of between about 0.1-2 micron. In an embodiment, the activated carbon filter disclosed above has a nominal micron rating of between about 0.2-2 micron. In an embodiment, the activated carbon filter disclosed above has a nominal micron rating of between about 0.3-2 micron. In an embodiment, the activated carbon filter disclosed above has a nominal micron rating of between about 0.4-2 micron. In an embodiment, the activated carbon filter disclosed above has a nominal micron rating of between about 0.5-2 micron. In an embodiment, the activated carbon filter disclosed above has a nominal micron rating of between about 0.6-2 micron, about 0.7-2 micron, about 0.8-2 micron. In an embodiment, the activated carbon filter disclosed above has a nominal micron rating of between about 0.9-2 micron. In an embodiment, the activated carbon filter disclosed above has a nominal micron rating of between about 1-2 micron. In an embodiment, the activated carbon filter disclosed above has a nominal micron rating of between about 1.25-2 micron. In an embodiment, the activated carbon filter disclosed above has a nominal micron rating of between about 1.5-2 micron. In an embodiment, the activated carbon filter disclosed above has a nominal micron rating of between about 1.75-2 micron.

In an embodiment, the activated carbon filter disclosed above has a nominal micron rating of between about 0.01-1 micron. In an embodiment, the activated carbon filter disclosed above has a nominal micron rating of between about 0.05-1 micron. In an embodiment, the activated carbon filter disclosed above has a nominal micron rating of between about 0.1-1 micron. In an embodiment, the activated carbon filter disclosed above has a nominal micron rating of between about 0.2-1 micron. In an embodiment, the activated carbon filter disclosed above has a nominal micron rating of between about 0.3-1 micron. In an embodiment, the activated carbon filter disclosed above has a nominal micron rating of between about 0.4-1 micron. In an embodiment, the activated carbon filter disclosed above has a nominal micron rating of between about 0.5-1 micron. In an embodiment, the activated carbon filter disclosed above has a nominal micron rating of between about 0.6-1 micron, about 0.7-1 micron, about 0.8-1 micron. In an embodiment, the activated carbon filter disclosed above has a nominal micron rating of between about 0.9-1 micron. Any number within any of the above ranges is contemplated as an embodiment of the disclosure.

In an embodiment, the activated carbon filtration step is conducted at a feed rate of between 1-500 LMH. In an embodiment, the activated carbon filtration step is conducted at a feed rate of between 10-500 LMH. In an embodiment, the activated carbon filtration step is conducted at a feed rate of between 20-500 LMH. In an embodiment, the activated carbon filtration step is conducted at a feed rate of between 30-500 LMH. In an embodiment, the activated carbon filtration step is conducted at a feed rate of between 50-500 LMH. In an embodiment, the activated carbon filtration step is conducted at a feed rate of between 100-500 LMH. In an embodiment, the activated carbon filtration step is conducted at a feed rate of between 150-500 LMH. In an embodiment, the activated carbon filtration step is conducted at a feed rate of between 200-500 LMH. In an embodiment, the activated carbon filtration step is conducted at a feed rate of between 300-500 LMH. In an embodiment, the activated carbon filtration step is conducted at a feed rate of between 400-500 LMH.

In an embodiment, the activated carbon filtration step is conducted at a feed rate of between 1-200 LMH. In an embodiment, the activated carbon filtration step is conducted at a feed rate of between 10-200 LMH. In an embodiment, the activated carbon filtration step is conducted at a feed rate of between 15-200 LMH. In an embodiment, the activated carbon filtration step is conducted at a feed rate of between 20-200 LMH. In an embodiment, the activated carbon filtration step is conducted at a feed rate of between 30-200 LMH. In an embodiment, the activated carbon filtration step is conducted at a feed rate of between 40-200 LMH. In an embodiment, the activated carbon filtration step is conducted at a feed rate of between 50-200 LMH. In an embodiment, the activated carbon filtration step is conducted at a feed rate of between 100-200 LMH. In an embodiment, the activated carbon filtration step is conducted at a feed rate of between 150-200 LMH. In an embodiment, the activated carbon filtration step is conducted at a feed rate of between 1-150 LMH. In an embodiment, the activated carbon filtration step is conducted at a feed rate of between 10-150 LMH. In an embodiment, the activated carbon filtration step is conducted at a feed rate of between 15-150 LMH. In an embodiment, the activated carbon filtration step is conducted at a feed rate of between 20-150 LMH. In an embodiment, the activated carbon filtration step is conducted at a feed rate of between 25-150 LMH. In an embodiment, the activated carbon filtration step is conducted at a feed rate of between 30-150 LMH. In an embodiment, the activated carbon filtration step is conducted at a feed rate of between 50-150 LMH. In an embodiment, the activated carbon filtration step is conducted at a feed rate of between 100-150 LMH.

In an embodiment, the activated carbon filtration step is conducted at a feed rate of between 1-100 LMH. In an embodiment, the activated carbon filtration step is conducted at a feed rate of between 10-100 LMH. In an embodiment, the activated carbon filtration step is conducted at a feed rate of between 15-100 LMH. In an embodiment, the activated carbon filtration step is conducted at a feed rate of between 20-100 LMH. In an embodiment, the activated carbon filtration step is conducted at a feed rate of between 30-100 LMH. In an embodiment, the activated carbon filtration step is conducted at a feed rate of between 40-100 LMH. In an embodiment, the activated carbon filtration step is conducted at a feed rate of between 50-100 LMH.

In an embodiment, the activated carbon filtration step is conducted at a feed rate of between 1-75 LMH. In an embodiment, the activated carbon filtration step is conducted at a feed rate of between 5-75 LMH. In an embodiment, the activated carbon filtration step is conducted at a feed rate of between 10-75 LMH. In an embodiment, the activated carbon filtration step is conducted at a feed rate of between 20-75 LMH. In an embodiment, the activated carbon filtration step is conducted at a feed rate of between 25-75 LMH. In an embodiment, the activated carbon filtration step is conducted at a feed rate of between 30-75 LMH. In an embodiment, the activated carbon filtration step is conducted at a feed rate of between 40-75 LMH. In an embodiment, the activated carbon filtration step is conducted at a feed rate of between 50-75 LMH. In an embodiment, the activated carbon filtration step is conducted at a feed rate of between 60-75 LMH. In an embodiment, the activated carbon filtration step is conducted at a feed rate of between 70-75 LMH.

In an embodiment, the activated carbon filtration step is conducted at a feed rate of between 1-50 LMH. In an embodiment, the activated carbon filtration step is conducted at a feed rate of between 5-50 LMH. In an embodiment, the activated carbon filtration step is conducted at a feed rate of between 10-50 LMH. In an embodiment, the activated carbon filtration step is conducted at a feed rate of between 20-50 LMH. In an embodiment, the activated carbon filtration step is conducted at a feed rate of between 25-50 LMH. In an embodiment, the activated carbon filtration step is conducted at a feed rate of between 30-50 LMH In an embodiment, the activated carbon filtration step is conducted at a feed rate of between 40-50 LMH.

Any whole number integer within any of the above ranges is contemplated as an embodiment of the disclosure.

In an embodiment, the activated carbon filtration step is conducted at a feed rate of about 1, about 2, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 300, about 350, about 400, about 450, about 500, about 550, about 600, about 700, about 800, about 900, about 950 or about 1000 LMH.

In an embodiment, the solution is treated by an activated carbon filter wherein the filter has a filter capacity of between 5-1000 L/m². In an embodiment, the solution is treated by an activated carbon filter wherein the filter has a filter capacity of between 10-750 L/m². In an embodiment, the solution is treated by an activated carbon filter wherein the filter has a filter capacity of between 15-500 L/m². In an embodiment, the solution is treated by an activated carbon filter wherein the filter has a filter capacity of between 20-400 L/m². In an embodiment, the solution is treated by an activated carbon filter wherein the filter has a filter capacity of between 25-300 L/m². In an embodiment, the solution is treated by an activated carbon filter wherein the filter has a filter capacity of between 30-250 L/m². In an embodiment, the solution is treated by an activated carbon filter wherein the filter has a filter capacity of between 40-200 L/m². In an embodiment, the solution is treated by an activated carbon filter wherein the filter has a filter capacity of between 30-100 L/m². Any number within any of the above ranges is contemplated as an embodiment of the disclosure.

In an embodiment, the solution is treated by an activated carbon filter wherein the filter has a filter capacity of about 5 L/m². In an embodiment, the solution is treated by an activated carbon filter wherein the filter has a filter capacity of about 10 L/m². In an embodiment, the solution is treated by an activated carbon filter wherein the filter has a filter capacity of about 15 L/m². In an embodiment, the solution is treated by an activated carbon filter wherein the filter has a filter capacity of about 20 L/m². In an embodiment, the solution is treated by an activated carbon filter wherein the filter has a filter capacity of about 25 L/m². In an embodiment, the solution is treated by an activated carbon filter wherein the filter has a filter capacity of about 30 L/m². In an embodiment, the solution is treated by an activated carbon filter wherein the filter has a filter capacity of about 40 L/m². In an embodiment, the solution is treated by an activated carbon filter wherein the filter has a filter capacity of about 50 L/m². In an embodiment, the solution is treated by an activated carbon filter wherein the filter has a filter capacity of about 75 L/m². In an embodiment, the solution is treated by an activated carbon filter wherein the filter has a filter capacity of about 100 L/m². In an embodiment, the solution is treated by an activated carbon filter wherein the filter has a filter capacity of about 150 L/m². In an embodiment, the solution is treated by an activated carbon filter wherein the filter has a filter capacity of about 200 L/m². In an embodiment, the solution is treated by an activated carbon filter wherein the filter has a filter capacity of about 300 L/m². In an embodiment, the solution is treated by an activated carbon filter wherein the filter has a filter capacity of about 400 L/m². In an embodiment, the solution is treated by an activated carbon filter wherein the filter has a filter capacity of about 500 L/m². In an embodiment, the solution is treated by an activated carbon filter wherein the filter has a filter capacity of about 1000 L/m².

If the content of contaminants is above the fixed threshold after a first activated carbon filtration step, the said step can be repeated. In an embodiment of the present invention, 1, 2, 3, 4, 5, 6, 7, 8, 9, or 10 activated carbon filtration step(s) are performed. In an embodiment of the present invention, 1, 2 or 3 activated carbon filtration step(s) are performed. In an embodiment of the present invention, 1 or 2 activated carbon filtration step(s) are performed. In an embodiment of the present invention, 2 activated carbon filtration step(s) is performed. In an embodiment of the present invention, 3 activated carbon filtration step(s) is performed. In an embodiment of the present invention, 4 activated carbon filtration step(s) is performed. In an embodiment of the present invention, activated carbon filtration step(s) is performed.

In an embodiment of the present invention, 1 activated carbon filtration step(s) is performed.

In an embodiment, the solution is treated by activated carbon filters in series. In an embodiment, the solution is treated by 1, 2, 3, 4, 5, 6, 7, 8, 9, or 10 activated carbon filters in series. In an embodiment, the solution is treated by 2, 3, 4 or 5 activated carbon filters in series. In an embodiment, the solution is treated by 2 activated carbon filters in series. In an embodiment, the solution is treated by 3 activated carbon filters in series. In an embodiment, the solution is treated by 4 activated carbon filters in series. In an embodiment, the solution is treated by 5 activated carbon filters in series.

In an embodiment the activated carbon filtration step is performed in a single pass mode. In another embodiment the activated carbon filtration step is performed in recirculation mode. In said embodiment (recirculation mode) 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 cycles of activated carbon filtration are performed. In another embodiment 2, 3, 4, 5, 6, 7, 8, 9 or 10 cycles of activated carbon filtration are performed. In an embodiment, 2 or 3 cycles of activated carbon filtration are performed. In an embodiment, 2 cycles of activated carbon filtration are performed. In an embodiment, 3 cycles of activated carbon filtration are performed. In an embodiment, 4 cycles of activated carbon filtration are performed. In an embodiment, 5 cycles of activated carbon filtration are performed.

1.10 Ultrafiltration and/or Diafiltration

In an embodiment, once the solution has been clarified by any of the method of section 1.7 above, the solution obtained (i.e. the supernatant) can optionally be further clarified by Ultrafiltration and/or Diafiltration.

In another embodiment, once the solution has been neutralized by any of the method of section 1.8 above, the solution obtained can optionally be further clarified by Ultrafiltration and/or Diafiltration.

In another embodiment, once the solution has been further clarified by any of the method of section 1.9 above, the solution obtained (i.e. the filtrate) can optionally be further clarified by Ultrafiltration and/or Diafiltration.

In another embodiment, once the solution has been neutralized by any of the method of section 1.8 above and clarified by the filtration step of section 1.9 above, the solution obtained (i.e. the filtrate) can optionally be further clarified by Ultrafiltration and/or Diafiltration.

Ultrafiltration (UF) is a process for concentrating a dilute product stream. UF separates molecules in solution based on the membrane pore size or molecular weight cutoff (MWCO).

In an embodiment of the present invention, the solution (e.g. the solution obtained at section 1.7, 1.8 or 1.9 above) is treated by ultrafiltration.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-750 kDa. In an embodiment the molecular weight cut off of the membrane is in the range of between about 10 kDa-500 kDa. In an embodiment the molecular weight cut off of the membrane is in the range of between about 10 kDa-300 kDa. In an embodiment the molecular weight cut off of the membrane is in the range of between about 10 kDa-100 kDa. In an embodiment the molecular weight cut off of the membrane is in the range of between about 10 kDa-50 kDa. In an embodiment the molecular weight cut off of the membrane is in the range of between about 10 kDa-30 kDa.

In an embodiment, the solution is treated by ultrafiltration and the molecular weight cut off of the membrane is in the range of between about 5 kDa-1000 kDa. In an embodiment the molecular weight cut off of the membrane is in the range of between about 10 kDa-1000 kDa. In an embodiment the molecular weight cut off of the membrane is in the range of between about 20 kDa-1000 kDa. In an embodiment the molecular weight cut off of the membrane is in the range of between about 30 kDa-1000 kDa. In an embodiment the molecular weight cut off of the membrane is in the range of between about 40 kDa-1000 kDa. In an embodiment the molecular weight cut off of the membrane is in the range of between about 50 kDa-1000 kDa. In an embodiment the molecular weight cut off of the membrane is in the range of between about 75 kDa-1000 kDa. In an embodiment the molecular weight cut off of the membrane is in the range of between about 75 kDa-1000 kDa. In an embodiment the molecular weight cut off of the membrane is in the range of between about 100 kDa-1000 kDa. In an embodiment the molecular weight cut off of the membrane is in the range of between about 150 kDa-1000 kDa. In an embodiment the molecular weight cut off of the membrane is in the range of between about 200 kDa-1000 kDa. In an embodiment the molecular weight cut off of the membrane is in the range of between about 300 kDa-1000 kDa. In an embodiment the molecular weight cut off of the membrane is in the range of between about 400 kDa-1000 kDa. In an embodiment the molecular weight cut off of the membrane is in the range of between about 500 kDa-1000 kDa. In an embodiment the molecular weight cut off of the membrane is in the range of between about 750 kDa-1000 kDa.

In an embodiment, the solution is treated by ultrafiltration and the molecular weight cut off of the membrane is in the range of between about 5 kDa-500 kDa. In an embodiment the molecular weight cut off of the membrane is in the range of between about 10 kDa-500 kDa. In an embodiment the molecular weight cut off of the membrane is in the range of between about 20 kDa-500 kDa. In an embodiment the molecular weight cut off of the membrane is in the range of between about 30 kDa-500 kDa. In an embodiment the molecular weight cut off of the membrane is in the range of between about 40 kDa-500 kDa. In an embodiment the molecular weight cut off of the membrane is in the range of between about 50 kDa-500 kDa. In an embodiment the molecular weight cut off of the membrane is in the range of between about 75 kDa-500 kDa. In an embodiment the molecular weight cut off of the membrane is in the range of between about 75 kDa-500 kDa. In an embodiment the molecular weight cut off of the membrane is in the range of between about 100 kDa-500 kDa. In an embodiment the molecular weight cut off of the membrane is in the range of between about 150 kDa-500 kDa. In an embodiment the molecular weight cut off of the membrane is in the range of between about 200 kDa-500 kDa. In an embodiment the molecular weight cut off of the membrane is in the range of between about 300 kDa-500 kDa. In an embodiment the molecular weight cut off of the membrane is in the range of between about 400 kDa-500 kDa.

In an embodiment, the solution is treated by ultrafiltration and the molecular weight cut off of the membrane is in the range of between about 5 kDa-300 kDa. In an embodiment the molecular weight cut off of the membrane is in the range of between about 10 kDa-300 kDa. In an embodiment the molecular weight cut off of the membrane is in the range of between about 20 kDa-300 kDa. In an embodiment the molecular weight cut off of the membrane is in the range of between about 30 kDa-300 kDa. In an embodiment the molecular weight cut off of the membrane is in the range of between about 40 kDa-300 kDa. In an embodiment the molecular weight cut off of the membrane is in the range of between about 50 kDa-300 kDa. In an embodiment the molecular weight cut off of the membrane is in the range of between about 75 kDa-300 kDa. In an embodiment the molecular weight cut off of the membrane is in the range of between about 75 kDa-300 kDa. In an embodiment the molecular weight cut off of the membrane is in the range of between about 100 kDa-300 kDa. In an embodiment the molecular weight cut off of the membrane is in the range of between about 150 kDa-300 kDa. In an embodiment the molecular weight cut off of the membrane is in the range of between about 200 kDa-300 kDa.

In an embodiment, the solution is treated by ultrafiltration and the molecular weight cut off of the membrane is in the range of between about 5 kDa-100 kDa. In an embodiment the molecular weight cut off of the membrane is in the range of between about 10 kDa-100 kDa. In an embodiment the molecular weight cut off of the membrane is in the range of between about 20 kDa-100 kDa. In an embodiment the molecular weight cut off of the membrane is in the range of between about 30 kDa-100 kDa. In an embodiment the molecular weight cut off of the membrane is in the range of between about 40 kDa-100 kDa. In an embodiment the molecular weight cut off of the membrane is in the range of between about 50 kDa-100 kDa. In an embodiment the molecular weight cut off of the membrane is in the range of between about 75 kDa-100 kDa. In an embodiment the molecular weight cut off of the membrane is in the range of between about 75 kDa-100 kDa.

In an embodiment the molecular weight cut off of the membrane is about 5 kDa. In an embodiment the molecular weight cut off of the membrane is about 10 kDa. In an embodiment the molecular weight cut off of the membrane is about 20 kDa. In an embodiment the molecular weight cut off of the membrane is about 30 kDa. In an embodiment the molecular weight cut off of the membrane is about 40 kDa. In an embodiment the molecular weight cut off of the membrane is about 50 kDa. In an embodiment the molecular weight cut off of the membrane is about 60 kDa. In an embodiment the molecular weight cut off of the membrane is about 70 kDa. In an embodiment the molecular weight cut off of the membrane is about 80 kDa. In an embodiment the molecular weight cut off of the membrane is about 90 kDa. In an embodiment the molecular weight cut off of the membrane is about 100 kDa. In an embodiment the molecular weight cut off of the membrane is about 110 kDa. In an embodiment the molecular weight cut off of the membrane is about 120 kDa. In an embodiment the molecular weight cut off of the membrane is about 130 kDa. In an embodiment the molecular weight cut off of the membrane is about 140 kDa. In an embodiment the molecular weight cut off of the membrane is about 150 kDa. In an embodiment the molecular weight cut off of the membrane is about 200 kDa. In an embodiment the molecular weight cut off of the membrane is about 250 kDa. In an embodiment the molecular weight cut off of the membrane is about 300 kDa. In an embodiment the molecular weight cut off of the membrane is about 400 kDa. In an embodiment the molecular weight cut off of the membrane is about 500 kDa. In an embodiment the molecular weight cut off of the membrane is about 5 kDa. In an embodiment the molecular weight cut off of the membrane is about 750 kDa. In an embodiment the molecular weight cut off of the membrane is about 1000 kDa.

In an embodiment, the concentration factor of the ultrafiltration step is from about 1.5 to 10. In an embodiment, the concentration factor is from about 3 to 9. In an embodiment, the concentration factor is from about 5 to 9. In an embodiment, the concentration factor is from about 2 to 8. In an embodiment, the concentration factor is from about 2 to 5.

In an embodiment, the concentration factor is about 1.5 In an embodiment, the concentration factor is about 2.0. In an embodiment, the concentration factor is about 3.0.

In an embodiment, the concentration factor is about 3.5. In an embodiment, the concentration factor is about 4.0. In an embodiment, the concentration factor is about 4.5.

In an embodiment, the concentration factor is about 5.0. In an embodiment, the concentration factor is about 5.5. In an embodiment, the concentration factor is about 6.0.

In an embodiment, the concentration factor is about 6.5. In an embodiment, the concentration factor is about 7.0. In an embodiment, the concentration factor is about 7.5.

In an embodiment, the concentration factor is about 8.0. In an embodiment, the concentration factor is about 8.5. In an embodiment, the concentration factor is about 9.0. In an embodiment, the concentration factor is about 9.5. In an embodiment, the concentration factor is about 10.0.

In an embodiment of the present invention, the solution (e.g. the solution obtained at section 1.7, 1.8 or 1.9 above) is treated by diafiltration.

In an embodiment of the present invention, the solution obtained following ultrafiltration (UF) as disclosed in the present section above is further treated by diafiltration (UF/DF treatment).

Diafiltration (DF) is used to exchange product into a desired buffer solution (or water only). In an embodiment, diafiltration is used to change the chemical properties of the retained solution under constant volume. Unwanted particles pass through a membrane while the make-up of the feed stream is changed to a more desirable state through the addition of a replacement solution (a buffer solution, a saline solution, a buffer saline solution or water).

In an embodiment, the replacement solution is water.

In an embodiment, the replacement solution is saline in water. In some embodiments, the salt is selected from the group consisting of magnesium chloride, potassium chloride, sodium chloride and a combination thereof. In some embodiments, the salt is magnesium chloride, potassium chloride, sodium chloride or a combination thereof. In one particular embodiment, the salt is magnesium chloride. In one particular embodiment, the salt is potassium chloride. In one particular embodiment, the salt is sodium chloride. In one particular embodiment, the salt is sodium chloride. In one embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM or about 500 mM. In one particular embodiment, the replacement solution is sodium chloride at about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM or about 300 mM. In one embodiment, the replacement solution is sodium chloride at about 1 mM. In one embodiment, the replacement solution is sodium chloride at about 5 mM. In one embodiment, the replacement solution is sodium chloride at about 10 mM. In one embodiment, the replacement solution is sodium chloride at about 20 mM. In one embodiment, the replacement solution is sodium chloride at about 30 mM. In one embodiment, the replacement solution is sodium chloride at about 40 mM. In one embodiment, the replacement solution is sodium chloride at about 50 mM. In one embodiment, the replacement solution is sodium chloride at about 60 mM. In one embodiment, the replacement solution is sodium chloride at about 70 mM. In one embodiment, the replacement solution is sodium chloride at about 80 mM. In one embodiment, the replacement solution is sodium chloride at about 90 mM. In one embodiment, the replacement solution is sodium chloride at about 100 mM. In one embodiment, the replacement solution is sodium chloride at about 110 mM. In one embodiment, the replacement solution is sodium chloride at about 120 mM. In one embodiment, the replacement solution is sodium chloride at about 150 mM. In one embodiment, the replacement solution is sodium chloride at about 200 mM. In one embodiment, the replacement solution is sodium chloride at about 250 mM. In one embodiment, the replacement solution is sodium chloride at about 300 mM. In one embodiment, the replacement solution is sodium chloride at about 350 mM. In one embodiment, the replacement solution is sodium chloride at about 400 mM. In one embodiment, the replacement solution is sodium chloride at about 450 mM. In one embodiment, the replacement solution is sodium chloride at about 500 mM.

In an embodiment, the replacement solution is a buffer solution. In an embodiment, the replacement solution is a buffer solution wherein the buffer is selected from the group consisting of N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) and Tris(hydroxymethyl)-aminomethane (Tris). In an embodiment, the replacement solution is a buffer solution wherein the buffer is N-(2-Acetamido)-aminoethanesulfonic acid (ACES), a salt of acetic acid (acetate), N-(2-Acetamido)-iminodiacetic acid (ADA), 2-Aminoethanesulfonic acid (AES, Taurine), ammonia, 2-Amino-2-methyl-1-propanol (AMP), 2-Amino-2-methyl-1,3-propanediol AMPD, ammediol, N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium hydrogen carbonate (bicarbonate), N,N′-Bis(2-hydroxyethyl)-glycine (bicine), [Bis-(2-hydroxyethyl)-imino]-tris-(hydroxymethylmethane) (BIS-Tris), 1,3-Bis[tris(hydroxymethyl)-methylamino]propane (BIS-Tris-Propane), Boric acid, dimethylarsinic acid (Cacodylate), 3-(Cyclohexylamino)-propanesulfonic acid (CAPS), 3-(Cyclohexylamino)-2-hydroxy-1-propanesulfonic acid (CAPSO), sodium carbonate (Carbonate), cyclohexylaminoethanesulfonic acid (CHES), a salt of citric acid (citrate), 3-[N-Bis(hydroxyethyl)amino]-2-hydroxypropanesulfonic acid (DIPSO), a salt of formic acid (formate), Glycine, Glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), imidazole, a salt of malic acid (Malate), a salt of maleic acid (Maleate), 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), a salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, a salt of succinic acid (Succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine) or Tris(hydroxymethyl)-aminomethane (Tris).

In an embodiment, the diafiltration buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate). In an embodiment, the diafiltration buffer is a salt of acetic acid (acetate), a salt of citric acid (citrate), a salt of formic acid (formate), a salt of malic acid (Malate), a salt of maleic acid (Maleate), a salt of phosphoric acid (Phosphate) or a salt of succinic acid (Succinate). In an embodiment, the diafiltration buffer is a salt of citric acid (citrate). In an embodiment, the diafiltration buffer is a salt of succinic acid (Succinate). In an embodiment, said salt is a sodium salt. In an embodiment, said salt is a potassium salt.

In an embodiment, the pH of the diafiltration buffer is between about 4.0-11.0. In an embodiment, the pH of the diafiltration buffer is between about 5.0-10.0. In an embodiment, the pH of the diafiltration buffer is between about 5.5-9.0. In an embodiment, the pH of the diafiltration buffer is between about 6.0-8.0. In an embodiment, the pH of the diafiltration buffer is between about 6.0-7.0. In an embodiment, the pH of the diafiltration buffer is between about 6.5-7.5. In an embodiment, the pH of the diafiltration buffer is between about 6.5-7.0. In an embodiment, the pH of the diafiltration buffer is between about 6.0-7.5. Any number within any of the above ranges is contemplated as an embodiment of the disclosure.

In an embodiment, the pH of the diafiltration buffer is about 4.0. In an embodiment, the pH of the diafiltration buffer is about 4.5. In an embodiment, the pH of the diafiltration buffer is about 5.0. In an embodiment, the pH of the diafiltration buffer is about 5.5. In an embodiment, the pH of the diafiltration buffer is about 6.0. In an embodiment, the pH of the diafiltration buffer is about 6.5. In an embodiment, the pH of the diafiltration buffer is about 7.0. In an embodiment, the pH of the diafiltration buffer is about 7.5. In an embodiment, the pH of the diafiltration buffer is about 8.0. In an embodiment, the pH of the diafiltration buffer is about 8.5. In an embodiment, the pH of the diafiltration buffer is about 9.0. In an embodiment, the pH of the diafiltration buffer is about 9.5. In an embodiment, the pH of the diafiltration buffer is about 10.0. In an embodiment, the pH of the diafiltration buffer is about 11.0. In an embodiment, the pH of the diafiltration buffer is about 7.0.

In an embodiment, the concentration of the diafiltration buffer is between about 0.01 mM-100 mM. In an embodiment, the concentration of the diafiltration buffer is between about 0.1 mM-100 mM. In an embodiment, the concentration of the diafiltration buffer is between about 0.5 mM-100 mM. In an embodiment, the concentration of the diafiltration buffer is between about 1 mM-100 mM. In an embodiment, the concentration of the diafiltration buffer is between about 2 mM-100 mM. In an embodiment, the concentration of the diafiltration buffer is between about 5 mM-100 mM. In an embodiment, the concentration of the diafiltration buffer is between about 10 mM-100 mM. In an embodiment, the concentration of the diafiltration buffer is between about 15 mM-100 mM. In an embodiment, the concentration of the diafiltration buffer is between about 20 mM-100 mM. In an embodiment, the concentration of the diafiltration buffer is between about 30 mM-100 mM. In an embodiment, the concentration of the diafiltration buffer is between about 40 mM-100 mM. In an embodiment, the concentration of the diafiltration buffer is between about 50 mM-100 mM. In an embodiment, the concentration of the diafiltration buffer is between about 75 mM-100 mM. In an embodiment, the concentration of the diafiltration buffer is between about 80 mM-100 mM. In an embodiment, the concentration of the diafiltration buffer is between about 85 mM-100 mM. In an embodiment, the concentration of the diafiltration buffer is between about 90 mM-100 mM. In an embodiment, the concentration of the diafiltration buffer is between about 95 mM-100 mM.

In an embodiment, the concentration of the diafiltration buffer is between about 0.01 mM-50 mM. In an embodiment, the concentration of the diafiltration buffer is between about 0.1 mM-50 mM. In an embodiment, the concentration of the diafiltration buffer is between about 0.5 mM-50 mM. In an embodiment, the concentration of the diafiltration buffer is between about 1 mM-50 mM. In an embodiment, the concentration of the diafiltration buffer is between about 5 mM-50 mM. In an embodiment, the concentration of the diafiltration buffer is between about 10 mM-50 mM. In an embodiment, the concentration of the diafiltration buffer is between about 15 mM-50 mM. In an embodiment, the concentration of the diafiltration buffer is between about 20 mM-50 mM. In an embodiment, the concentration of the diafiltration buffer is between about 25 mM-50 mM. In an embodiment, the concentration of the diafiltration buffer is between about 30 mM-50 mM. In an embodiment, the concentration of the diafiltration buffer is between about 40 mM-50 mM. In an embodiment, the concentration of the diafiltration buffer is between about 45 mM-50 mM.

In an embodiment, the concentration of the diafiltration buffer is between about 0.01 mM-25 mM. In an embodiment, the concentration of the diafiltration buffer is between about 0.1 mM-25 mM. In an embodiment, the concentration of the diafiltration buffer is between about 0.5 mM-25 mM. In an embodiment, the concentration of the diafiltration buffer is between about 1 mM-25 mM. In an embodiment, the concentration of the diafiltration buffer is between about 5 mM-25 mM. In an embodiment, the concentration of the diafiltration buffer is between about 10 mM-25 mM. In an embodiment, the concentration of the diafiltration buffer is between about 15 mM-25 mM. In an embodiment, the concentration of the diafiltration buffer is between about 20 mM-25 mM.

In an embodiment, the concentration of the diafiltration buffer is between about 0.01 mM-15 mM. In an embodiment, the concentration of the diafiltration buffer is between about 0.1 mM-15 mM. In an embodiment, the concentration of the diafiltration buffer is between about 0.5 mM-15 mM. In an embodiment, the concentration of the diafiltration buffer is between about 1 mM-15 mM. In an embodiment, the concentration of the diafiltration buffer is between about 5 mM-15 mM. In an embodiment, the concentration of the diafiltration buffer is between about 10 mM-15 mM.

In an embodiment, the concentration of the diafiltration buffer is between about 0.01 mM-10 mM. In an embodiment, the concentration of the diafiltration buffer is between about 0.1 mM-10 mM. In an embodiment, the concentration of the diafiltration buffer is between about 0.5 mM-10 mM. In an embodiment, the concentration of the diafiltration buffer is between about 1 mM-10 mM. In an embodiment, the concentration of the diafiltration buffer is between about 5 mM-10 mM.

Any number within any of the above ranges is contemplated as an embodiment of the disclosure.

In an embodiment, the concentration of the diafiltration buffer is about 0.01 mM. In an embodiment, the concentration of the diafiltration buffer is about 0.05 mM. In an embodiment, the concentration of the diafiltration buffer is about 0.1 mM. In an embodiment, the concentration of the diafiltration buffer is about 0.5 mM. In an embodiment, the concentration of the diafiltration buffer is about 1 mM. In an embodiment, the concentration of the diafiltration buffer is about 5 mM. In an embodiment, the concentration of the diafiltration buffer is about 10 mM. In an embodiment, the concentration of the diafiltration buffer is about 15 mM. In an embodiment, the concentration of the diafiltration buffer is about 20 mM. In an embodiment, the concentration of the diafiltration buffer is about 30 mM. In an embodiment, the concentration of the diafiltration buffer is about 40 mM. In an embodiment, the concentration of the diafiltration buffer is about 50 mM. In an embodiment, the concentration of the diafiltration buffer is about 75 mM. In an embodiment, the concentration of the diafiltration buffer is about 100 mM.

In an embodiment, the concentration of the diafiltration buffer is about 20 mM.

In an embodiment, the replacement solution comprises a chelating agent. In an embodiment, the replacement solution comprises an alum chelating agent. In some embodiments, the chelating agent is selected from the group consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) and combinations of these. In some embodiments, the chelating agent is Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N,N,N′,N″,N″-pentaacetic acid (DTPA), 1,3-diaminopropan-2-ol-N,N,N′,N′-tetraacetic acid (DPTA-OH), ethylenediamine-N,N′-bis(2-hydroxyphenylacetic acid) (EDDHA), ethylenediamine-N,N′-dipropionic acid dihydrochloride (EDDP), ethylenediamine-tetrakis(methylenesulfonic acid) (EDTPO), Nitrilotris(methylenephosphonic acid) (NTPO), imino-diacetic acid (IDA), hydroxyimino-diacetic acid (HIDA), nitrilo-triacetic acid (NTP), triethylenetetramine-hexaacetic acid (TTHA), Dimercaptosuccinic acid (DMSA), 2,3-dimercapto-1-propanesulfonic acid (DMPS), alpha lipoic acid (ALA), Nitrilotriacetic acid (NTA), thiamine tetrahydrofurfuryl disulfide (TTFD), dimercaprol, penicillamine, deferoxamine (DFOA), deferasirox, phosphonates, a salt of citric acid (citrate) or combinations of these.

In some embodiments, the chelating agent is selected from the group consisting of Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N, N, N′,N″,N″-pentaacetic acid (DTPA), 1,3-diaminopropan-2-ol-N, N, N′,N′-tetraacetic acid (DPTA-OH), ethylenediamine-N,N′-bis(2-hydroxyphenylacetic acid) (EDDHA), a salt of citric acid (citrate) and combinations of these. In some embodiments, the chelating agent is Ethylene Diamine Tetra Acetate (EDTA), N-(2-Hydroxyethyl)ethylenediamine-N,N′,N′-triacetic acid (EDTA-OH), hydroxy ethylene diamine triacetic acid (HEDTA), Ethylene glycol-bis(2-aminoethylether)-N,N,N′,N′-tetraacetic acid (EGTA), 1,2-cyclohexanediamine-N,N,N′,N′-tetraacetic acid (CyDTA), diethylenetriamine-N, N, N′,N″,N″-pentaacetic acid (DTPA), 1,3-diaminopropan-2-ol-N, N, N′,N′-tetraacetic acid (DPTA-OH), ethylenediamine-N,N′-bis(2-hydroxyphenylacetic acid) (EDDHA), a salt of citric acid (citrate) or combinations of these.

In some embodiments, the chelating agent is N-(2-Hydroxyethyl)ethylenediamine-N,N′,N′-triacetic acid (EDTA-OH). In some embodiments, the chelating agent is hydroxy ethylene diamine triacetic acid (HEDTA). In some embodiments, the chelating agent is Ethylene glycol-bis(2-aminoethylether)-N,N,N′,N′-tetraacetic acid (EGTA).

In some embodiments, the chelating agent is 1,2-cyclohexanediamine-N,N,N′,N′-tetraacetic acid (CyDTA). In some embodiments, the chelating agent is diethylenetriamine-N,N,N′,N″,N″-pentaacetic acid (DTPA). In some embodiments, the chelating agent is 1,3-diaminopropan-2-ol-N, N,N′,N′-tetraacetic acid (DPTA-OH). In some embodiments, the chelating agent is ethylenediamine-N,N′-bis(2-hydroxyphenylacetic acid) (EDDHA).

In some embodiments, the chelating agent is Ethylene Diamine Tetra Acetate (EDTA).

In some embodiments, the chelating agent is a salt of citric acid (citrate). In some embodiments, the chelating agent is sodium citrate.

In general, the chelating agent is employed at a concentration from 1 to 500 mM. In an embodiment, the concentration of the chelating agent in the replacement solution is from 2 to 400 mM. In an embodiment, the concentration of the chelating agent in the replacement solution is from 10 to 400 mM. In an embodiment, the concentration of the chelating agent in the replacement solution is from 10 to 200 mM. In an embodiment, the concentration of the chelating agent in the replacement solution is from 10 to 100 mM. In an embodiment, the concentration of the chelating agent in the replacement solution is from 10 to 50 mM. In an embodiment, the concentration of the chelating agent in the replacement solution is from 10 to 30 mM.

In an embodiment, the concentration of the chelating agent in the replacement solution is about 0.01 mM. In an embodiment, the concentration of the chelating agent in the replacement solution is about 0.05 mM. In an embodiment, the concentration of the chelating agent in the replacement solution is about 0.1 mM. In an embodiment, the concentration of the chelating agent in the replacement solution is about 0.5 mM. In an embodiment, the concentration of the chelating agent in the replacement solution is about 1 mM. In an embodiment, the concentration of the chelating agent in the replacement solution is about 5 mM. In an embodiment, the concentration of the chelating agent in the replacement solution is about 10 mM. In an embodiment, the concentration of the chelating agent in the replacement solution is about 15 mM. In an embodiment, the concentration of the chelating agent in the replacement solution is about 20 mM. In an embodiment, the concentration of the chelating agent in the replacement solution is about 25 mM. In an embodiment, the concentration of the chelating agent in the replacement solution is about 30 mM. In an embodiment, the concentration of the chelating agent in the replacement solution is about 40 mM. In an embodiment, the concentration of the chelating agent in the replacement solution is about 50 mM. In an embodiment, the concentration of the chelating agent in the replacement solution is about 60 mM. In an embodiment, the concentration of the chelating agent in the replacement solution is about 75 mM. In an embodiment, the concentration of the chelating agent in the replacement solution is about 100 mM.

In an embodiment, the diafiltration buffer solution comprises a salt. In some embodiments, the salt is selected from the group consisting of magnesium chloride, potassium chloride, sodium chloride and a combination thereof. In some embodiments, the salt is magnesium chloride, potassium chloride, sodium chloride or a combination thereof. In one particular embodiment, the salt is magnesium chloride. In one particular embodiment, the salt is potassium chloride. In one particular embodiment, the salt is sodium chloride. In an embodiment, the diafiltration buffer solution comprises sodium chloride at about 1, about 5, about 10, about 15, about 20, about 25, about 30, about 35, about 40, about 45, about 50, about 55, about 60, about 65, about 70, about 80, about 90, about 100, about 110, about 120, about 130, about 140, about 150, about 160, about 170, about 180, about 190, about 200, about 250, about 300, about 350, about 400, about 450 or about 500 mM.

In one particular embodiment, the diafiltration buffer solution comprises sodium chloride at about 1 mM. In one particular embodiment, the diafiltration buffer solution comprises sodium chloride at about 5 mM. In one particular embodiment, the diafiltration buffer solution comprises sodium chloride at about 10 mM. In one particular embodiment, the diafiltration buffer solution comprises sodium chloride at about 20 mM. In one particular embodiment, the diafiltration buffer solution comprises sodium chloride at about 30 mM. In one particular embodiment, the diafiltration buffer solution comprises sodium chloride at about 40 mM. In one particular embodiment, the diafiltration buffer solution comprises sodium chloride at about 45 mM. In one particular embodiment, the diafiltration buffer solution comprises sodium chloride at about 50 mM. In one particular embodiment, the diafiltration buffer solution comprises sodium chloride at about 55 mM. In one particular embodiment, the diafiltration buffer solution comprises sodium chloride at about 60 mM. In one particular embodiment, the diafiltration buffer solution comprises sodium chloride at about 75 mM. In one particular embodiment, the diafiltration buffer solution comprises sodium chloride at about 100 mM. In one particular embodiment, the diafiltration buffer solution comprises sodium chloride at about 150 mM. In one particular embodiment, the diafiltration buffer solution comprises sodium chloride at about 200 mM. In one particular embodiment, the diafiltration buffer solution comprises sodium chloride at about 300 mM. In one particular embodiment, the diafiltration buffer solution comprises sodium chloride at about 50 mM

In an embodiment of the present invention, the number of diavolumes is at least 5, 10, 15, 20, 25, 30, 35, 40, 45, or 50. In an embodiment of the present invention, the number of diavolumes is between 5 and 30. In an embodiment of the present invention, the number of diavolumes is between 5 and 20. In an embodiment of the present invention, the number of diavolumes is between 5 and 10. In an embodiment of the present invention, the number of diavolumes is about 1. In an embodiment of the present invention, the number of diavolumes is about 2. In an embodiment of the present invention, the number of diavolumes is about 3. In an embodiment of the present invention, the number of diavolumes is about 4. In an embodiment of the present invention, the number of diavolumes is about 5. In an embodiment of the present invention, the number of diavolumes is about 6. In an embodiment of the present invention, the number of diavolumes is about 7. In an embodiment of the present invention, the number of diavolumes is about 8. In an embodiment of the present invention, the number of diavolumes is about 9. In an embodiment of the present invention, the number of diavolumes is about 10. In an embodiment of the present invention, the number of diavolumes is about 11. In an embodiment of the present invention, the number of diavolumes is about 12. In an embodiment of the present invention, the number of diavolumes is about 13. In an embodiment of the present invention, the number of diavolumes is about 14. In an embodiment of the present invention, the number of diavolumes is about 15. In an embodiment of the present invention, the number of diavolumes is about 20. In an embodiment of the present invention, the number of diavolumes is about 25. In an embodiment of the present invention, the number of diavolumes is about 30. In an embodiment of the present invention, the number of diavolumes is about 35. In an embodiment of the present invention, the number of diavolumes is about 40. In an embodiment of the present invention, the number of diavolumes is about 45. In an embodiment of the present invention, the number of diavolumes is about 50. In an embodiment of the present invention, the number of diavolumes is about 60. In an embodiment of the present invention, the number of diavolumes is about 70. In an embodiment of the present invention, the number of diavolumes is about 80. In an embodiment of the present invention, the number of diavolumes is about 90. In an embodiment of the present invention, the number of diavolumes is about 100.

In an embodiment of the present invention, the Ultrafiltration and Diafiltration steps are performed at a temperature between about 20° C. to about 90° C. In an embodiment, the Ultrafiltration and Diafiltration steps are performed at a temperature between about 35° C. to about 80° C. In an embodiment, the Ultrafiltration and Diafiltration steps are performed at temperature between about 40° C. to about 70° C. In an embodiment, the Ultrafiltration and Diafiltration steps are performed at temperature between about 45° C. to about 65° C. In an embodiment, the Ultrafiltration and Diafiltration steps are performed at temperature between about 50° C. to about 60° C. In an embodiment, the Ultrafiltration and Diafiltration steps are performed at temperature between about 50° C. to about 55° C. In an embodiment, the Ultrafiltration and Diafiltration steps are performed at temperature between about 45° C. to about 55° C. In an embodiment, the Ultrafiltration and Diafiltration steps are performed at temperature between about 45° C. to about 55° C.

Any number within any of the above ranges is contemplated as an embodiment of the disclosure.

In an embodiment, the Ultrafiltration and Diafiltration steps are performed at a temperature of about 20° C. In an embodiment, the Ultrafiltration and Diafiltration steps are performed at a temperature of about 25° C. In an embodiment, the Ultrafiltration and Diafiltration steps are performed at a temperature of about 30° C. In an embodiment, the Ultrafiltration and Diafiltration steps are performed at a temperature of about 35° C. In an embodiment, the Ultrafiltration and Diafiltration steps are performed at a temperature of about 40° C. In an embodiment, the Ultrafiltration and Diafiltration steps are performed at a temperature of about 45° C. In an embodiment, the Ultrafiltration and Diafiltration steps are performed at a temperature of about 50° C. In an embodiment, the Ultrafiltration and Diafiltration steps are performed at a temperature of about 55° C. In an embodiment, the Ultrafiltration and Diafiltration steps are performed at a temperature of about 60° C. In an embodiment, the Ultrafiltration and Diafiltration steps are performed at a temperature of about 65° C. In an embodiment, the Ultrafiltration and Diafiltration steps are performed at a temperature of about 70° C. In an embodiment, the Ultrafiltration and Diafiltration steps are performed at a temperature of about 75° C. In an embodiment, the Ultrafiltration and Diafiltration steps are performed at a temperature of about 80° C. In an embodiment, the Ultrafiltration and Diafiltration steps are performed at a temperature of about 50° C.

In an embodiment of the present invention, the Diafiltration step is performed at a temperature between about 20° C. to about 90° C. In an embodiment, the Diafiltration step is performed at a temperature between about 35° C. to about 80° C. In an embodiment, the Diafiltration step is performed at temperature between about 40° C. to about 70° C. In an embodiment, the Diafiltration step is performed at temperature between about 45° C. to about 65° C. In an embodiment, the Diafiltration step is performed at temperature between about 50° C. to about 60° C. In an embodiment, the Diafiltration step is performed at temperature between about 50° C. to about 55° C. In an embodiment, the Diafiltration step is performed at temperature between about 45° C. to about 55° C. In an embodiment, the Diafiltration step is performed at temperature between about 45° C. to about 55° C.

Any number within any of the above ranges is contemplated as an embodiment of the disclosure.

In an embodiment, the Diafiltration step is performed at a temperature of about 20° C. In an embodiment, the Diafiltration step is performed at a temperature of about 25° C. In an embodiment, the Diafiltration step is performed at a temperature of about 30° C. In an embodiment, the Diafiltration step is performed at a temperature of about 35° C. In an embodiment, the Diafiltration step is performed at a temperature of about 40° C. In an embodiment, the Diafiltration step is performed at a temperature of about 45° C. In an embodiment, the Diafiltration step is performed at a temperature of about 50° C. In an embodiment, the Diafiltration step is performed at a temperature of about 55° C. In an embodiment, the Diafiltration step is performed at a temperature of about 60° C. In an embodiment, the Diafiltration step is performed at a temperature of about 65° C. In an embodiment, the Diafiltration step is performed at a temperature of about 70° C. In an embodiment, the Diafiltration step is performed at a temperature of about 75° C. In an embodiment, the Diafiltration step is performed at a temperature of about 80° C. In an embodiment, the Diafiltration step is performed at a temperature of about 50° C.

In an embodiment of the present invention, the Ultrafiltration step is performed at a temperature between about 20° C. to about 90° C. In an embodiment, the Ultrafiltration step is performed at a temperature between about 35° C. to about 80° C. In an embodiment, the Ultrafiltration step is performed at temperature between about 40° C. to about 70° C. In an embodiment, the Ultrafiltration step is performed at temperature between about 45° C. to about 65° C. In an embodiment, the Ultrafiltration step is performed at temperature between about 50° C. to about 60° C. In an embodiment, the Ultrafiltration step is performed at temperature between about 50° C. to about 55° C. In an embodiment, the Ultrafiltration step is performed at temperature between about 45° C. to about 55° C. In an embodiment, the Ultrafiltration step is performed at temperature between about 45° C. to about 55° C. Any number within any of the above ranges is contemplated as an embodiment of the disclosure.

In an embodiment, the Ultrafiltration step is performed at a temperature of about 20° C. In an embodiment, the Ultrafiltration step is performed at a temperature of about 25° C. In an embodiment, the Ultrafiltration step is performed at a temperature of about 30° C. In an embodiment, the Ultrafiltration step is performed at a temperature of about 35° C. In an embodiment, the Ultrafiltration step is performed at a temperature of about 40° C. In an embodiment, the Ultrafiltration step is performed at a temperature of about 45° C. In an embodiment, the Ultrafiltration step is performed at a temperature of about 50° C. In an embodiment, the Ultrafiltration step is performed at a temperature of about 55° C. In an embodiment, the Ultrafiltration step is performed at a temperature of about 60° C. In an embodiment, the Ultrafiltration step is performed at a temperature of about 65° C. In an embodiment, the Ultrafiltration step is performed at a temperature of about 70° C. In an embodiment, the Ultrafiltration step is performed at a temperature of about 75° C. In an embodiment, the Ultrafiltration step is performed at a temperature of about 80° C. In an embodiment, the Ultrafiltration step is performed at a temperature of about 50° C.

1.11 Homogenization/Sizing

A polysaccharide can become slightly reduced in size during the purification procedures. In an embodiment, the purified solution of polysaccharide of the present invention (e.g. obtained by Ultrafiltration and/or Diafiltration of section 1.10) is not sized.

In an embodiment, the polysaccharide can be homogenized by sizing techniques. Mechanical or chemical sizing maybe employed. Chemical hydrolysis maybe conducted using for example acetic acid. Mechanical sizing maybe conducted using High Pressure Homogenization Shearing.

Therefore, in an embodiment, the purified solution of polysaccharide obtained (e.g. obtained by Ultrafiltration and/or Diafiltration of section 1.10) is sized to a target molecular weight.

As used herein, the term “molecular weight” of polysaccharide refers to molecular weight calculated for example by size exclusion chromatography (SEC) combined with multiangle laser light scattering detector (MALLS).

In some embodiments, the purified polysaccharide is sized to a molecular weight of between about 5 kDa and about 4,000 kDa. In other such embodiments, the purified polysaccharide is sized to a molecular weight of between about 10 kDa and about 4,000 kDa. In other such embodiments, the purified polysaccharide is sized to a molecular weight of between about 50 kDa and about 4,000 kDa. In further such embodiments, the purified polysaccharide is sized to a molecular weight of between about 50 kDa and about 3,500 kDa. In further such embodiments, the purified polysaccharide is sized to a molecular weight of between about 50 kDa and about 3,000 kDa. In further such embodiments, the purified polysaccharide is sized to a molecular weight of between about 50 kDa and about 2,500 kDa. In further such embodiments, the purified polysaccharide is sized to a molecular weight of between about 50 kDa and about 2,000 kDa. In further such embodiments, the purified polysaccharide is sized to a molecular weight of between about 50 kDa and about 1,750 kDa. In further such embodiments, the purified polysaccharide is sized to a molecular weight of about between about 50 kDa and about 1,500 kDa. In further such embodiments, the purified polysaccharide is sized to a molecular weight of between about 50 kDa and about 1,250 kDa. In further such embodiments, the purified polysaccharide is sized to a molecular weight of between about 50 kDa and about 1,000 kDa. In further such embodiments, the purified polysaccharide is sized to a molecular weight of between about 50 kDa and about 750 kDa. In further such embodiments, the purified polysaccharide is sized to a molecular weight of between about 50 kDa and about 500 kDa. In further such embodiments, the purified polysaccharide is sized to a molecular weight of between about 100 kDa and about 4,000 kDa. In further such embodiments, the purified polysaccharide is sized to a molecular weight of between about 100 kDa and about 3,500 kDa. In further such embodiments, the purified polysaccharide is sized to a molecular weight of about 100 kDa and about 3,000 kDa. In further such embodiments, the purified polysaccharide is sized to a molecular weight of about 100 kDa and about 2,500 kDa. In further such embodiments, the purified polysaccharide is sized to a molecular weight of about 100 kDa and about 2,250 kDa. In further such embodiments, the purified polysaccharide is sized to a molecular weight of between about 100 kDa and about 2,000 kDa. In further such embodiments, the purified polysaccharide is sized to a molecular weight of between about 100 kDa and about 1,750 kDa. In further such embodiments, the purified polysaccharide is sized to a molecular weight of between about 100 kDa and about 1,500 kDa. In further such embodiments, the purified polysaccharide is sized to a molecular weight of between about 100 kDa and about 1,250 kDa. In further such embodiments, the purified polysaccharide is sized to a molecular weight of between about 100 kDa and about 1,000 kDa. In further such embodiments, the purified polysaccharide is sized to a molecular weight of between about 100 kDa and about 750 kDa. In further such embodiments, the purified polysaccharide is sized to a molecular weight of between about 100 kDa and about 500 kDa. In further such embodiments, the purified polysaccharide is sized to a molecular weight of between about 200 kDa and about 4,000 kDa. In further such embodiments, the purified polysaccharide is sized to a molecular weight of between about 200 kDa and about 3,500 kDa. In further such embodiments, the purified polysaccharide is sized to a molecular weight of between about 200 kDa and about 3,000 kDa. In further such embodiments, the purified polysaccharide is sized to a molecular weight of between about 200 kDa and about 2,500 kDa. In further such embodiments, the purified polysaccharide is sized to a molecular weight of between about 200 kDa and about 2,250 kDa. In further such embodiments, the purified polysaccharide is sized to a molecular weight of between about 200 kDa and about 2,000 kDa. In further such embodiments, the purified polysaccharide is sized to a molecular weight of between about 200 kDa and about 1,750 kDa. In further such embodiments, the purified polysaccharide is sized to a molecular weight of between about 200 kDa and about 1,500 kDa. In further such embodiments, the purified polysaccharide is sized to a molecular weight of between about 200 kDa and about 1,250 kDa. In further such embodiments, the purified polysaccharide is sized to a molecular weight of between about 200 kDa and about 1,000 kDa. In further such embodiments, the purified polysaccharide is sized to a molecular weight of between about 200 kDa and about 750 kDa. In further such embodiments, the purified polysaccharide is sized to a molecular weight of between about 200 kDa and about 500 kDa. In further such embodiments, the purified polysaccharide is sized to a molecular weight of between about 250 kDa and about 1,000 kDa. In further such embodiments, the purified polysaccharide is sized to a molecular weight of between about 250 kDa and about 750 kDa. In further such embodiments, the purified polysaccharide is sized to a molecular weight of between about 250 kDa and about 500 kDa. In further such embodiments, the purified polysaccharide is sized to a molecular weight of between about 300 kDa and about 1,000 kDa. In further such embodiments, the purified polysaccharide is sized to a molecular weight of between about 300 kDa and about 750 kDa. In further such embodiments, the purified polysaccharide is sized to a molecular weight of between about 300 kDa and about 500 kDa. In further such embodiments, the purified polysaccharide is sized to a molecular weight of between about 500 kDa and about 1,500 kDa. In further such embodiments, the purified polysaccharide is sized to a molecular weight of between about 500 kDa and about 1,250 kDa. In further such embodiments, the purified polysaccharide is sized to a molecular weight of between about 500 kDa and about 1,000 kDa. In further such embodiments, the purified polysaccharide is sized to a molecular weight of between about 500 kDa and about 750 kDa. In further such embodiments, the purified polysaccharide is sized to a molecular weight of between about 500 kDa and about 600 kDa.

Any number within any of the above ranges is contemplated as an embodiment of the disclosure.

1.12 Sterile Filtration

In an embodiment, the purified solution of polysaccharide of the invention is sterilely filtered.

Therefore, in an embodiment, the Ultrafiltration and/or Diafiltration step of section 1.10 can optionally be followed by a sterile filtration step.

In an embodiment, the homogenizing/sizing step of section 1.11 if conducted can optionally be followed by a sterile filtration step.

In an embodiment, any of the step of sections 1.2 to 1.9 can optionally be followed by a sterile filtration step.

In an embodiment, the step of section 1.2 is followed by a sterile filtration step. In an embodiment, the step of section 1.3 is followed by a sterile filtration step. In an embodiment, the step of section 1.4 is followed by a sterile filtration step. In an embodiment, the step of section 1.5 is followed by a sterile filtration step. In an embodiment, the step of section 1.6 is followed by a sterile filtration step. In an embodiment, the step of section 1.7 is followed by a sterile filtration step. In an embodiment, the step of section 1.8 is followed by a sterile filtration step. In an embodiment, the step of section 1.9 is followed by a sterile filtration step.

In an embodiment, sterile filtration is dead-end filtration (perpendicular filtration). In an embodiment, sterile filtration is tangential filtration.

In an embodiment, the solution is treated by a sterile filtration step wherein the filter has a nominal retention range of between about 0.01-0.2 micron. In an embodiment, the solution is treated by a sterile filtration step wherein the filter has a nominal retention range of between about 0.05-0.2 micron. In an embodiment, the solution is treated by a sterile filtration step wherein the filter has a nominal retention range of between about 0.1-0.2 micron. In an embodiment, the solution is treated by a sterile filtration step wherein the filter has a nominal retention range of between about 0.15-0.2 micron.

Any number within any of the above ranges is contemplated as an embodiment of the disclosure.

In an embodiment, the solution is treated by a sterile filtration step wherein the filter has a nominal retention range of about 0.05 micron. In an embodiment, the solution is treated by a sterile filtration step wherein the filter has a nominal retention range of about 0.1 micron. In an embodiment, the solution is treated by a sterile filtration step wherein the filter has a nominal retention range of about 0.15 micron. In an embodiment, the solution is treated by a sterile filtration step wherein the filter has a nominal retention range of about 0.2 micron.

In an embodiment, the solution is treated by a sterile filtration step wherein the filter has a nominal retention range of about 0.2 micron.

In an embodiment, the solution is treated by a sterile filtration step wherein the filter has a filter capacity of about 25-1500 L/m². In an embodiment, the solution is treated by a sterile filtration step wherein the filter has a filter capacity of 50-1500 L/m². In an embodiment, the solution is treated by a sterile filtration step wherein the filter has a filter capacity of 75-1500 L/m². In an embodiment, the solution is treated by a sterile filtration step wherein the filter has a filter capacity of 100-1500 L/m². In an embodiment, the solution is treated by a sterile filtration step wherein the filter has a filter capacity of 250-1500 L/m². In an embodiment, the solution is treated by a sterile filtration step wherein the filter has a filter capacity of 500-1500 L/m². In an embodiment, the solution is treated by a sterile filtration step wherein the filter has a filter capacity of 750-1500 L/m². In an embodiment, the solution is treated by a sterile filtration step wherein the filter has a filter capacity of 1000-1500 L/m². In an embodiment, the solution is treated by a sterile filtration step wherein the filter has a filter capacity of 1250-1500 L/m².

In an embodiment, the solution is treated by a sterile filtration step wherein the filter has a filter capacity of about 25-1000 L/m². In an embodiment, the solution is treated by a sterile filtration step wherein the filter has a filter capacity of 50-1000 L/m². In an embodiment, the solution is treated by a sterile filtration step wherein the filter has a filter capacity of 75-1000 L/m². In an embodiment, the solution is treated by a sterile filtration step wherein the filter has a filter capacity of 100-1000 L/m². In an embodiment, the solution is treated by a sterile filtration step wherein the filter has a filter capacity of 250-1000 L/m². In an embodiment, the solution is treated by a sterile filtration step wherein the filter has a filter capacity of 400-1000 L/m². In an embodiment, the solution is treated by a sterile filtration step wherein the filter has a filter capacity of 500-1000 L/m². In an embodiment, the solution is treated by a sterile filtration step wherein the filter has a filter capacity of 750-1000 L/m².

In an embodiment, the solution is treated by a sterile filtration step wherein the filter has a filter capacity of 25-500 L/m². In an embodiment, the solution is treated by a sterile filtration step wherein the filter has a filter capacity of 50-500 L/m². In an embodiment, the solution is treated by a sterile filtration step wherein the filter has a filter capacity of 75-500 L/m². In an embodiment, the solution is treated by a sterile filtration step wherein the filter has a filter capacity of 100-500 L/m². In an embodiment, the solution is treated by a sterile filtration step wherein the filter has a filter capacity of 250-500 L/m². In an embodiment, the solution is treated by a sterile filtration step wherein the filter has a filter capacity of 300-500 L/m². In an embodiment, the solution is treated by a sterile filtration step wherein the filter has a filter capacity of 400-500 L/m².

In an embodiment, the solution is treated by a sterile filtration step wherein the filter has a filter capacity of 25-250 L/m². In an embodiment, the solution is treated by a sterile filtration step wherein the filter has a filter capacity of 50-250 L/m². In an embodiment, the solution is treated by a sterile filtration step wherein the filter has a filter capacity of 75-250 L/m². In an embodiment, the solution is treated by a sterile filtration step wherein the filter has a filter capacity of 100-250 L/m². In an embodiment, the solution is treated by a sterile filtration step wherein the filter has a filter capacity of 150-250 L/m². In an embodiment, the solution is treated by a sterile filtration step wherein the filter has a filter capacity of 200-250 L/m².

In an embodiment, the solution is treated by a sterile filtration step wherein the filter has a filter capacity of 25-100 L/m². In an embodiment, the solution is treated by a sterile filtration step wherein the filter has a filter capacity of 50-100 L/m². In an embodiment, the solution is treated by a sterile filtration step wherein the filter has a filter capacity of 75-100 L/m².

Any number within any of the above ranges is contemplated as an embodiment of the disclosure.

In an embodiment, the solution is treated by a microfiltration step wherein the filter has a filter capacity of about 25 L/m². In an embodiment, the solution is treated by a microfiltration step wherein the filter has a filter capacity of about 50 L/m². In an embodiment, the solution is treated by a microfiltration step wherein the filter has a filter capacity of about 75 L/m². In an embodiment, the solution is treated by a microfiltration step wherein the filter has a filter capacity of about 100 L/m². In an embodiment, the solution is treated by a microfiltration step wherein the filter has a filter capacity of about 150 L/m². In an embodiment, the solution is treated by a microfiltration step wherein the filter has a filter capacity of about 200 L/m². In an embodiment, the solution is treated by a microfiltration step wherein the filter has a filter capacity of about 250 L/m². In an embodiment, the solution is treated by a microfiltration step wherein the filter has a filter capacity of about 300 L/m². In an embodiment, the solution is treated by a microfiltration step wherein the filter has a filter capacity of about 400 L/m². In an embodiment, the solution is treated by a microfiltration step wherein the filter has a filter capacity of about 500 L/m². In an embodiment, the solution is treated by a microfiltration step wherein the filter has a filter capacity of about 750 L/m². In an embodiment, the solution is treated by a microfiltration step wherein the filter has a filter capacity of about 1000 L/m². In an embodiment, the solution is treated by a microfiltration step wherein the filter has a filter capacity of about 1500 L/m².

1.13 Final Material

The purified S. pneumoniae serotype 3 polysaccharide can be finally prepared as a liquid solution.

The polysaccharide can be further processed (e.g. lyophilized as a dried powder, see WO2006/110381). Therefore, in an embodiment, the polysaccharide is a dried powder.

In an embodiment, the polysaccharide is a freeze-dried cake.

2 Uses of the Purified S. pneumoniae Serotype 3 Polysaccharide

The S. pneumoniae serotype 3 polysaccharide purified by the method of the present invention may be used as an antigen. Plain polysaccharides are used as antigens in vaccines (see the 23-valent unconjugated pneumococcal polysaccharide vaccine Pneumovax).

The S. pneumoniae serotype 3 polysaccharide purified by the method of the present invention may also be conjugated to carrier protein(s) to obtain a glycoconjugate.

2.1 Glycoconjugates

The S. pneumoniae serotype 3 polysaccharide purified by the method of the present invention may be conjugated to carrier protein(s) to obtain a glycoconjugate.

For the purposes of the invention the term ‘glycoconjugate’ indicates a saccharide covalently linked to a carrier protein. In one embodiment a saccharide is linked directly to a carrier protein. In a second embodiment a saccharide is linked to a carrier protein through a spacer/linker.

In general, covalent conjugation of saccharides to carriers enhances the immunogenicity of saccharides as it converts them from T-independent antigens to T-dependent antigens, thus allowing priming for immunological memory. Conjugation is particularly useful for pediatric vaccines.

Purified polysaccharides by the method of the invention may be activated (e.g., chemically activated) to make them capable of reacting (e.g. with a linker or directly with the carrier protein) and then incorporated into glycoconjugates, as further described herein.

The purified polysaccharide maybe sized to a target molecular weight before conjugation e.g. by the methods disclosed at section 1.11 above. Therefore, in an embodiment, the purified polysaccharide is sized before conjugation. In an embodiment, the purified polysaccharide as disclosed herein may be sized before conjugation to obtain an oligosaccharide. Oligosaccharides have a low number of repeat units (typically 5-15 repeat units) and are typically derived by sizing (e.g. hydrolysis) of the polysaccharide. Preferably though, the saccharide to be used for conjugation is a polysaccharide. High molecular weight polysaccharides are able to induce certain antibody immune responses due to the epitopes present on the antigenic surface. The isolation and purification of high molecular weight polysaccharides is preferably contemplated for use in the conjugates of the present invention.

Therefore, in an embodiment, the S. pneumoniae serotype 3 polysaccharide is sized and remains a polysaccharide.

In an embodiment, the polysaccharide is not sized.

In some embodiments, the purified polysaccharide before conjugation (after sizing or unsized) has a molecular weight of between 5 kDa and 4,000 kDa. In other such embodiments, the purified polysaccharide has a molecular weight of between 10 kDa and 4,000 kDa. In other such embodiments, the purified polysaccharide has a molecular weight of between 50 kDa and 4,000 kDa. In further such embodiments, the purified polysaccharide has a molecular weight of between about 50 kDa and about 3,500 kDa. In further such embodiments, the purified polysaccharide has a molecular weight of between about 50 kDa and about 3,000 kDa. In further such embodiments, the purified polysaccharide has a molecular weight of between about 50 kDa and about 2,500 kDa. In further such embodiments, the purified polysaccharide has a molecular weight of between about 50 kDa and about 2,000 kDa. In further such embodiments, the purified polysaccharide has a molecular weight of between about 50 kDa and about 1,750 kDa. In further such embodiments, the purified polysaccharide has a molecular weight of about between about 50 kDa and about 1,500 kDa. In further such embodiments, the purified polysaccharide has a molecular weight of between about 50 kDa and about 1,250 kDa. In further such embodiments, the purified polysaccharide has a molecular weight of between about 50 kDa and about 1,000 kDa.

Any number within any of the above ranges is contemplated as an embodiment of the disclosure.

Any suitable conjugation reaction can be used, with any suitable linker where necessary. See for example WO2007116028 pages 17-22.

The purified oligosaccharides or polysaccharides described herein are chemically activated to make the saccharides capable of reacting with the carrier protein.

In an embodiment, the glycoconjugate is prepared using reductive amination.

Reductive amination involves two steps, (1) oxidation (activation) of the purified saccharide, (2) reduction of the activated saccharide and a carrier protein (e.g., CRM₁₉₇, DT, TT or PD) to form a glycoconjugate (see e.g. WO2015110941, WO2015110940). As mentioned above, before oxidation, sizing of the polysaccharide to a target molecular weight (MW) range can be performed. Mechanical or chemical hydrolysis may be employed. Chemical hydrolysis may be conducted using acetic acid. In an embodiment, the size of the purified polysaccharide is reduced by mechanical homogenization.

In an embodiment, the purified polysaccharide or oligosaccharide is conjugated to a carrier protein by a process comprising the step of:
(a) reacting said purified polysaccharide or oligosaccharide with an oxidizing agent;
(b) optionally quenching the oxidation reaction by addition of a quenching agent;
(c) compounding the activated polysaccharide or oligosaccharide of step (a) or (b) with a carrier protein; and
(d) reacting the compounded activated polysaccharide or oligosaccharide and carrier protein with a reducing agent to form a glycoconjugate.

Following the oxidation step (a) the saccharide is said to be activated and is referred to as “activated polysaccharide or oligosaccharide”.

The oxidation step (a) may involve reaction with periodate. For the purpose of the present invention, the term “periodate” includes both periodate and periodic acid; the term also includes both metaperiodate (IO₄_{^{−}}) and orthoperiodate (IO₆_{^{5−}}) and the various salts of periodate (e.g., sodium periodate and potassium periodate).

In a preferred embodiment, the oxidizing agent is sodium periodate. In an embodiment, the periodate used for the oxidation is metaperiodate. In an embodiment the periodate used for the oxidation is sodium metaperiodate.

The oxidation step (a) may involve reaction with a stable nitroxyl or nitroxide radical compound, such as piperidine-N-oxy or pyrrolidine-N-oxy compounds, in the presence of an oxidant to selectively oxidize primary hydroxyls of the said polysaccharide or oligosaccharide to produce an activated saccharide containing aldehyde groups (see WO2014097099). In an aspect, said stable nitroxyl or nitroxide radical compound is any one as disclosed at page 3 line 14 to page 4 line 7 of WO2014097099 and the oxidant is any one as disclosed at page 4 line 8 to 15 of WO2014097099. In an aspect, said stable nitroxyl or nitroxide radical compound is 2,2,6,6-tetramethyl-1-piperidinyloxy (TEMPO) and the oxidant is N-chlorosuccinimide (NCS).

In one embodiment, the quenching agent is as disclosed in WO2015110941 (see page line 3 to 26).

In an embodiment, the reduction reaction (d) is carried out in aqueous solvent. In an embodiment, the reduction reaction (d) is carried out in aprotic solvent. In an embodiment, the reduction reaction (d) is carried out in DMSO (dimethylsulfoxide) or in DMF (dimethylformamide)) solvent.

In an embodiment, the reducing agent is sodium cyanoborohydride, sodium triacetoxyborohydride, sodium or zinc borohydride in the presence of Bronsted or Lewis acids, amine boranes such as pyridine borane, 2-Picoline Borane, 2,6-diborane-methanol, dimethylamine-borane, t-BuMeⁱPrN—BH₃, benzylamine-BH₃ or 5-ethyl-2-methylpyridine borane (PEMB). In a preferred embodiment, the reducing agent is sodium cyanoborohydride.

At the end of the reduction reaction, there may be unreacted aldehyde groups remaining in the conjugates, these may be capped using a suitable capping agent. In one embodiment this capping agent is sodium borohydride (NaBH₄).

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

In an embodiment, the glycoconjugate is prepared using cyanylation chemistry.

In an embodiment, the purified polysaccharide or oligosaccharide is activated with cyanogen bromide. The activation corresponds to cyanylation of the hydroxyl groups of the polysaccharide or oligosaccharide. The activated polysaccharide or oligosaccharide is then coupled directly or via a spacer (linker) group to an amino group on the carrier protein.

In an embodiment, the purified polysaccharide or oligosaccharide is activated with 1-cyano-4-dimethylamino pyridinium tetrafluoroborate (CDAP) to form a cyanate ester. The activated polysaccharide or oligosaccharide is then coupled directly or via a spacer (linker) group to an amino group on the carrier protein.

In an embodiment, the spacer could be cystamine or cysteamine to give a thiolated polysaccharide or oligosaccharide which could be coupled to the carrier via a thioether linkage obtained after reaction with a maleimide-activated carrier protein (for example using N-[γ-maleimidobutyrloxy]succinimide ester (GMBS)) or a haloacetylated carrier protein (for example using iodoacetimide, N-succinimidyl bromoacetate (SBA; SIB), N-succinimidyl(4-iodoacetyl)aminobenzoate (SIAB), sulfosuccinimidyl(4-iodoacetyl)aminobenzoate (sulfo-SIAB), N-succinimidyl iodoacetate (SIA) or succinimidyl 3-[bromoacetamido]proprionate (SBAP)). Preferably, the cyanate ester (optionally made by CDAP chemistry) is coupled with hexane diamine or adipic acid dihydrazide (ADH) and the amino-derivatised saccharide is conjugated to the carrier protein (e.g., CRM₁₉₇) using carbodiimide (e.g., EDAC or EDC) chemistry via a carboxyl group on the protein carrier. Such conjugates are described for example in WO 93/15760, WO 95/08348 and WO 96/129094.

In an embodiment, the glycoconjugate is prepared by a method of making glycoconjugates as disclosed in WO2014027302. The resulting glycoconjugate comprises a saccharide covalently conjugated to a carrier protein through a bivalent, heterobifunctional spacer (2-((2-oxoethyl)thio)ethyl)carbamate (eTEC). Alternatively, the the glycoconjugate is prepared by a method of making glycoconjugates as disclosed in WO2015121783.

Other suitable conjugation techniques use carbodiimides (e.g. EDC (1-Ethyl-3-(3-dimethylaminopropyl) carbodiimide hydrochloride, EDC plus Sulfo NHS, CMC (1-Cyclohexyl-3-(2-morpholinoethyl)carbodiimide, DCC (N,N′-Dicyclohexyl carbodiimide), or DIC (diisopropyl carbodiimide).

In an embodiment, the polysaccharide or oligosaccaride is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is adipic acid dihydrazide (ADH). Other linkers include B-propionamido (WO 00/10599), nitrophenyl-ethylamine, haloalkyl halide), glycosidic linkages (U.S. Pat. Nos. 4,673,574, 4,808,700), hexane diamine and 6-aminocaproic acid (U.S. Pat. No. 4,459,286).

Carrier Protein

A component of the glycoconjugate is a carrier protein to which the purified polysaccharide or oligosaccharide is conjugated. The terms “protein carrier” or “carrier protein” or “carrier” may be used interchangeably herein. Carrier proteins should be amenable to standard conjugation procedures.

In a preferred embodiment, the carrier protein of the glycoconjugate is selected in the group consisting of: DT (Diphtheria toxin), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA), recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). In an embodiment, the carrier protein of the glycoconjugate is DT (Diphtheria toxin), TT (tetanus toxoid) or fragment C of TT, CRM₁₉₇ (a nontoxic but antigenically identical variant of diphtheria toxin), other DT mutants (such as CRM₁₇₆, CRM₂₂₈, CRM₄₅ (Uchida et al. (1973) J. Biol. Chem. 218:3838-3844), CRM₉, CRM₁₀₂, CRM₁₀₃ or CRM₁₀₇; and other mutations described by Nicholls and Youle in Genetically Engineered Toxins, Ed: Frankel, Maecel Dekker Inc. (1992); deletion or mutation of Glu-148 to Asp, Gln or Ser and/or Ala 158 to Gly and other mutations disclosed in U.S. Pat. Nos. 4,709,017 and 4,950,740; mutation of at least one or more residues Lys 516, Lys 526, Phe 530 and/or Lys 534 and other mutations disclosed in U.S. Pat. Nos. 5,917,017 and 6,455,673; or fragment disclosed in U.S. Pat. No. 5,843,711, pneumococcal pneumolysin (ply) (Kuo et al. (1995) Infect Immun 63:2706-2713) including ply detoxified in some fashion, for example dPLY-GMBS (WO 2004/081515, WO 2006/032499) or dPLY-formol, PhtX, including PhtA, PhtB, PhtD, PhtE (sequences of PhtA, PhtB, PhtD or PhtE are disclosed in WO 00/37105 and WO 00/39299) and fusions of Pht proteins, for example PhtDE fusions, PhtBE fusions, Pht A-E (WO 01/98334, WO 03/054007, WO 2009/000826), OMPC (meningococcal outer membrane protein), which is usually extracted from Neisseria meningitidis serogroup B (EP0372501), PorB (from N. meningitidis), PD (Haemophilus influenzae protein D; see, e.g., EP0594610 B), or immunologically functional equivalents thereof, synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al. (2001) Eur J Immunol 31:3816-3824) such as N19 protein (Baraldoi et al. (2004) Infect Immun 72:4884-4887) pneumococcal surface protein PspA (WO 02/091998), iron uptake proteins (WO 01/72337), toxin A or B of Clostridium difficile (WO 00/61761), transferrin binding proteins, pneumococcal adhesion protein (PsaA) or recombinant Pseudomonas aeruginosa exotoxin A (in particular non-toxic mutants thereof (such as exotoxin A bearing a substution at glutamic acid 553 (Douglas et al. (1987) J. Bacteriol. 169(11):4967-4971)). Other proteins, such as ovalbumin, keyhole limpet hemocyanin (KLH), bovine serum albumin (BSA) or purified protein derivative of tuberculin (PPD) also can be used as carrier proteins. Other suitable carrier proteins include inactivated bacterial toxins such as cholera toxoid (e.g., as described in WO 2004/083251), Escherichia coli LT, E. coli ST, and exotoxin A from P. aeruginosa.

In a preferred embodiment, the carrier protein of the glycoconjugate is independently selected from the group consisting of TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile and PsaA. In another preferred embodiment, the carrier protein of the glycoconjugate is TT, DT, DT mutants (such as CRM₁₉₇), H. influenzae protein D, PhtX, PhtD, PhtDE fusions (particularly those described in WO 01/98334 and WO 03/054007), detoxified pneumolysin, PorB, N19 protein, PspA, OMPC, toxin A or B of C. difficile or PsaA.

In an embodiment, the carrier protein of the glycoconjugate is DT (Diphtheria toxoid). In another embodiment, the carrier protein of the glycoconjugate is TT (tetanus toxoid).

In another embodiment, the carrier protein of the glycoconjugate is PD (H. influenzae protein D; see, e.g., EP0594610 B).

In a preferred embodiment, the purified polysaccharide or oligosaccharide is conjugated to CRM₁₉₇ protein. The CRM₁₉₇ protein is a nontoxic form of diphtheria toxin but is immunologically indistinguishable from the diphtheria toxin. CRM₁₉₇ is produced by Corynebacterium diphtheriae infected by the nontoxigenic phage β197^{tox} created by nitrosoguanidine mutagenesis of the toxigenic corynephage beta (Uchida et al. (1971) Nature New Biology 233:8-11). The CRM₁₉₇ protein has the same molecular weight as the diphtheria toxin but differs therefrom by a single base change (guanine to adenine) in the structural gene. This single base change causes an amino acid substitution (glutamic acid for glycine) in the mature protein and eliminates the toxic properties of diphtheria toxin. The CRM₁₉₇ protein is a safe and effective T-cell dependent carrier for saccharides. Further details about CRM₁₉₇ and production thereof can be found, e.g., in U.S. Pat. No. 5,614,382.

In an embodiment, the purified polysaccharide or oligosaccharide is conjugated to CRM₁₉₇ protein or the A chain of CRM₁₉₇ (see CN103495161). In an embodiment, the purified polysaccharide or oligosaccharide is conjugated the A chain of CRM₁₉₇ obtained via expression by genetically recombinant E. coli (see CN103495161).

Preferably the ratio of carrier protein to polysaccharide or oligosaccharide in the glycoconjugate is between 1:5 and 5:1; e.g. between 1:0.5 and 4:1, between 1:1 and 3.5:1, between 1.2:1 and 3:1, between 1.5:1 and 2.5:1; e.g. between 1:2 and 2.5:1 or between 1:1 and 2:1 (w/w).

Following conjugation to the carrier protein, the glycoconjugate can be purified (enriched with respect to the amount of saccharide-protein conjugate) by a variety of techniques known to the skilled person. These techniques include dialysis, concentration/diafiltration operations, tangential flow filtration precipitation/elution, column chromatography (DEAE or hydrophobic interaction chromatography), and depth filtration.

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

2.2 Immunogenic Compositions

In an embodiment the invention relates to an immunogenic composition comprising any of the purified polysaccharide and/or glycoconjugate disclosed herein.

In an embodiment the invention relates to an immunogenic composition comprising any of the glycoconjugate disclosed herein.

In an embodiment the invention relates to an immunogenic composition comprising from 1 to 25 different glycoconjugates.

In an embodiment the invention relates to an immunogenic composition comprising from 26 to 45 different glycoconjugates.

In an embodiment the invention relates to an immunogenic composition comprising from 1 to 25 glycoconjugates from different serotypes of S. pneumoniae (1 to 25 pneumococcal conjugates). In one embodiment the invention relates to an immunogenic composition comprising glycoconjugates from 7, 8, 9, 10, 11, 12, 13, 14, 15, 16, 17, 18, 19, 20, 21, 22, 23, 24 or 25 different serotypes of S. pneumoniae. In one embodiment the immunogenic composition comprises glycoconjugates from 16 or 20 different serotypes of S. pneumoniae. In an embodiment the immunogenic composition is a 7, 8, 9, 10, 11, 12, 13, 14, 15, 16, 17, 18, 19 or 20-valent pneumococcal conjugate compositions. In an embodiment the immunogenic composition is a 14, 15, 16, 17, 18 or 19-valent pneumococcal conjugate compositions. In an embodiment the immunogenic composition is a 16-valent pneumococcal conjugate composition. In an embodiment the immunogenic composition is a 19-valent pneumococcal conjugate compositions. In an embodiment the immunogenic composition is a 20-valent pneumococcal conjugate composition.

In an embodiment said immunogenic composition comprises glycoconjugates from S. pneumoniae serotypes 3, 4, 6B, 9V, 14, 18C, 19F and 23F.

In an embodiment said immunogenic composition comprises in addition glycoconjugates from S. pneumoniae serotypes 1, 5 and 7F.

In an embodiment any of the immunogenic compositions above comprises in addition glycoconjugates from S. pneumoniae serotypes 6A and 19A.

In an embodiment any of the immunogenic compositions above comprise in addition a glycoconjugates from S. pneumoniae serotype 22F and 33F.

In an embodiment any of the immunogenic compositions above comprise in addition a glycoconjugates from S. pneumoniae serotypes 8, 10A, 11A, 12F and 15B.

In an embodiment any of the immunogenic compositions above comprise in addition a glycoconjugates from S. pneumoniae serotype 2.

In an embodiment any of the immunogenic compositions above comprise in addition a glycoconjugates from S. pneumoniae serotypes 9N.

In an embodiment any of the immunogenic compositions above comprise in addition a glycoconjugates from S. pneumoniae serotypes 17F.

In an embodiment any of the immunogenic compositions above comprise in addition a glycoconjugates from S. pneumoniae serotypes 20.

In a preferred embodiment, the saccharides are each individually conjugated to different molecules of the protein carrier (each molecule of protein carrier only having one type of saccharide conjugated to it). In said embodiment, the capsular saccharides are said to be individually conjugated to the carrier protein. Preferably, all the glycoconjugates of the above immunogenic compositions are individually conjugated to the carrier protein.

In an embodiment of any of the above immunogenic compositions, the glycoconjugate from S. pneumoniae serotype 3 is conjugated to CRM₁₉₇. In an embodiment of any of the above immunogenic compositions, the glycoconjugate from S. pneumoniae serotype 22F is conjugated to CRM₁₉₇. In an embodiment of any of the above immunogenic compositions, the glycoconjugate from S. pneumoniae serotype 33F is conjugated to CRM₁₉₇. In an embodiment of any of the above immunogenic compositions, the glycoconjugate from S. pneumoniae serotype 15B is conjugated to CRM₁₉₇. In an embodiment of any of the above immunogenic compositions, the glycoconjugate from S. pneumoniae serotype 12F is conjugated to CRM₁₉₇. In an embodiment of any of the above immunogenic compositions, the glycoconjugate from S. pneumoniae serotype 10A is conjugated to CRM₁₉₇. In an embodiment of any of the above immunogenic compositions, the glycoconjugate from S. pneumoniae serotype 11A is conjugated to CRM₁₉₇. In an embodiment of any of the above immunogenic compositions, the glycoconjugate from S. pneumoniae serotype 8 is conjugated to CRM₁₉₇. In an embodiment of any of the above immunogenic compositions, the glycoconjugates from S. pneumoniae serotypes 4, 6B, 9V, 14, 18C, 19F and 23F are conjugated to CRM₁₉₇. In an embodiment of any of the above immunogenic compositions, the glycoconjugates from S. pneumoniae serotypes 1, 5 and 7F are conjugated to CRM₁₉₇. In an embodiment of any of the above immunogenic compositions, the glycoconjugates from S. pneumoniae serotypes 6A and 19A are conjugated to CRM₁₉₇.

In an embodiment, the glycoconjugates of any of the above immunogenic compositions are all individually conjugated to CRM₁₉₇.

In an embodiment, the glycoconjugates from S. pneumoniae serotypes 1, 4, 5, 6B, 7F, 9V, 14 and/or 23F of any of the above immunogenic compositions are individually conjugated to PD.

In an embodiment, the glycoconjugate from S. pneumoniae serotype 18C of any of the above immunogenic compositions is conjugated to TT.

In an embodiment, the glycoconjugate from S. pneumoniae serotype 19F of any of the above immunogenic compositions is conjugated to DT.

In an embodiment, the glycoconjugates from S. pneumoniae serotypes 1, 4, 5, 6B, 7F, 9V, 14 and/or 23F of any of the above immunogenic compositions are individually conjugated to PD, the glycoconjugate from S. pneumoniae serotype 18C is conjugated to TT and the glycoconjugate from S. pneumoniae serotype 19F is conjugated to DT.

In an embodiment the above immunogenic compositions comprise from 8 to 20 different serotypes of S. pneumoniae.

In some embodiments, the immunogenic compositions disclosed herein may further comprise at least one, two or three adjuvants. In some embodiments, the immunogenic compositions disclosed herein may further comprise one adjuvant. The term “adjuvant” refers to a compound or mixture that enhances the immune response to an antigen. Antigens may act primarily as a delivery system, primarily as an immune modulator or have strong features of both. Suitable adjuvants include those suitable for use in mammals, including humans.

Examples of known suitable delivery-system type adjuvants that can be used in humans include, but are not limited to, alum (e.g., aluminum phosphate, aluminum sulfate or aluminum hydroxide), calcium phosphate, liposomes, oil-in-water emulsions such as MF59 (4.3% w/v squalene, 0.5% w/v polysorbate 80 (Tween 80), 0.5% w/v sorbitan trioleate (Span 85)), water-in-oil emulsions such as Montanide, and poly(D,L-lactide-co-glycolide) (PLG) microparticles or nanoparticles.

In an embodiment, the immunogenic compositions disclosed herein comprise aluminum salts (alum) as adjuvant (e.g., aluminum phosphate, aluminum sulfate or aluminum hydroxide). In a preferred embodiment, the immunogenic compositions disclosed herein comprise aluminum phosphate or aluminum hydroxide as adjuvant.

The immunogenic compositions may be formulated in liquid form (i.e., solutions or suspensions) or in a lyophilized form. Liquid formulations may advantageously be administered directly from their packaged form and are thus ideal for injection without the need for reconstitution in aqueous medium as otherwise required for lyophilized compositions of the invention.

Formulation of the immunogenic composition of the present disclosure can be accomplished using art-recognized methods. For instance, the individual polysaccharides and/or conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

The present disclosure provides an immunogenic composition comprising any of combination of polysaccharide or glycoconjugates disclosed herein and a pharmaceutically acceptable excipient, carrier, or diluent.

In an embodiment, the immunogenic composition of the disclosure is in liquid form, preferably in aqueous liquid form.

Immunogenic compositions of the disclosure may comprise one or more of a buffer, a salt, a divalent cation, a non-ionic detergent, a cryoprotectant such as a sugar, and an anti-oxidant such as a free radical scavenger or chelating agent, or any multiple combinations thereof.

In an embodiment, the immunogenic compositions of the disclosure comprise a buffer. In an embodiment, said buffer has a pKa of about 3.5 to about 7.5. In some embodiments, the buffer is phosphate, succinate, histidine or citrate. In certain embodiments, the buffer is succinate at a final concentration of 1 mM to 10 mM. In one particular embodiment, the final concentration of the succinate buffer is about 5 mM.

In an embodiment, the immunogenic compositions of the disclosure comprise a salt. In some embodiments, the salt is selected from the group consisting of magnesium chloride, potassium chloride, sodium chloride and a combination thereof. In some embodiments, the salt is magnesium chloride, potassium chloride, sodium chloride or a combination thereof. In one particular embodiment, the salt is sodium chloride. In one particular embodiment, the immunogenic compositions of the invention comprise sodium chloride at 150 mM.

In an embodiment, the immunogenic compositions of the disclosure comprise a surfactant. In an embodiment, the surfactant is selected from the group consisting of polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin and a poloxamer. In an embodiment, the surfactant is polysorbate 20 (TWEEN™20), polysorbate 40 (TWEEN™40), polysorbate 60 (TWEEN™60), polysorbate 65 (TWEEN™65), polysorbate 80 (TWEEN™80), polysorbate 85 (TWEEN™85), TRITON™ N-101, TRITON™ X-100, oxtoxynol 40, nonoxynol-9, triethanolamine, triethanolamine polypeptide oleate, polyoxyethylene-660 hydroxystearate (PEG-15, Solutol H 15), polyoxyethylene-35-ricinoleate (CREMOPHOR® EL), soy lecithin or a poloxamer. In one particular embodiment, the surfactant is polysorbate 80. In some said embodiment, the final concentration of polysorbate 80 in the formulation is at least 0.0001% to 10% polysorbate 80 weight to weight (w/w). In some said embodiments, the final concentration of polysorbate 80 in the formulation is at least 0.001% to 1% polysorbate 80 weight to weight (w/w). In some said embodiments, the final concentration of polysorbate 80 in the formulation is at least 0.01% to 1% polysorbate 80 weight to weight (w/w). In other embodiments, the final concentration of polysorbate 80 in the formulation is 0.01%, 0.02%, 0.03%, 0.04%, 0.05%, 0.06%, 0.07%, 0.08%, 0.09% or 0.1% polysorbate 80 (w/w). In another embodiment, the final concentration of the polysorbate 80 in the formulation is 0.02% polysorbate 80 (w/w). In another embodiment, the final concentration of the polysorbate 80 in the formulation is 0.01% polysorbate 80 (w/w). In another embodiment, the final concentration of the polysorbate 80 in the formulation is 0.03% polysorbate 80 (w/w). In another embodiment, the final concentration of the polysorbate 80 in the formulation is 0.04% polysorbate 80 (w/w). In another embodiment, the final concentration of the polysorbate 80 in the formulation is 0.05% polysorbate 80 (w/w). In another embodiment, the final concentration of the polysorbate 80 in the formulation is 1% polysorbate 80 (w/w). In one particular embodiment, the surfactant is polysorbate 20. In some said embodiment, the final concentration of polysorbate 20 in the formulation is at least 0.0001% to 10% polysorbate 20 weight to weight (w/w). In some said embodiments, the final concentration of polysorbate 20 in the formulation is at least 0.001% to 1% polysorbate 20 weight to weight (w/w). In some said embodiments, the final concentration of polysorbate 20 in the formulation is at least 0.01% to 1% polysorbate 20 weight to weight (w/w). In other embodiments, the final concentration of polysorbate 20 in the formulation is 0.01%, 0.02%, 0.03%, 0.04%, 0.05%, 0.06%, 0.07%, 0.08%, 0.09% or 0.1% polysorbate 20 (w/w). In another embodiment, the final concentration of the polysorbate 20 in the formulation is 0.02% polysorbate 20 (w/w). In another embodiment, the final concentration of the polysorbate 20 in the formulation is 0.01% polysorbate 20 (w/w). In another embodiment, the final concentration of the polysorbate 20 in the formulation is 0.03% polysorbate 20 (w/w). In another embodiment, the final concentration of the polysorbate 20 in the formulation is 0.04% polysorbate 80 (w/w). In another embodiment, the final concentration of the polysorbate 20 in the formulation is 0.05% polysorbate 20 (w/w). In another embodiment, the final concentration of the polysorbate 20 in the formulation is 1% polysorbate 20 (w/w).

In one particular embodiment, the surfactant is polysorbate 40. In some said embodiment, the final concentration of polysorbate 40 in the formulation is at least 0.0001% to 10% polysorbate 40 weight to weight (w/w). In some said embodiments, the final concentration of polysorbate 40 in the formulation is at least 0.001% to 1% polysorbate 40 weight to weight (w/w). In some said embodiments, the final concentration of polysorbate 40 in the formulation is at least 0.01% to 1% polysorbate 40 weight to weight (w/w). In other embodiments, the final concentration of polysorbate 40 in the formulation is 0.01%, 0.02%, 0.03%, 0.04%, 0.05%, 0.06%, 0.07%, 0.08%, 0.09% or 0.1% polysorbate 40 (w/w). In another embodiment, the final concentration of the polysorbate 40 in the formulation is 1% polysorbate 40 (w/w).

In one particular embodiment, the surfactant is polysorbate 60. In some said embodiment, the final concentration of polysorbate 60 in the formulation is at least 0.0001% to 10% polysorbate 60 weight to weight (w/w). In some said embodiments, the final concentration of polysorbate 60 in the formulation is at least 0.001% to 1% polysorbate 60 weight to weight (w/w). In some said embodiments, the final concentration of polysorbate 60 in the formulation is at least 0.01% to 1% polysorbate 60 weight to weight (w/w). In other embodiments, the final concentration of polysorbate 60 in the formulation is 0.01%, 0.02%, 0.03%, 0.04%, 0.05%, 0.06%, 0.07%, 0.08%, 0.09% or 0.1% polysorbate 60 (w/w). In another embodiment, the final concentration of the polysorbate 60 in the formulation is 1% polysorbate 60 (w/w).

In one particular embodiment, the surfactant is polysorbate 65. In some said embodiment, the final concentration of polysorbate 65 in the formulation is at least 0.0001% to 10% polysorbate 65 weight to weight (w/w). In some said embodiments, the final concentration of polysorbate 65 in the formulation is at least 0.001% to 1% polysorbate 65 weight to weight (w/w). In some said embodiments, the final concentration of polysorbate 65 in the formulation is at least 0.01% to 1% polysorbate 65 weight to weight (w/w). In other embodiments, the final concentration of polysorbate 65 in the formulation is 0.01%, 0.02%, 0.03%, 0.04%, 0.05%, 0.06%, 0.07%, 0.08%, 0.09% or 0.1% polysorbate 65 (w/w). In another embodiment, the final concentration of the polysorbate 65 in the formulation is 1% polysorbate 65 (w/w).

In one particular embodiment, the surfactant is polysorbate 85. In some said embodiment, the final concentration of polysorbate 85 in the formulation is at least 0.0001% to 10% polysorbate 85 weight to weight (w/w). In some said embodiments, the final concentration of polysorbate 85 in the formulation is at least 0.001% to 1% polysorbate 85 weight to weight (w/w). In some said embodiments, the final concentration of polysorbate 85 in the formulation is at least 0.01% to 1% polysorbate 85 weight to weight (w/w). In other embodiments, the final concentration of polysorbate 85 in the formulation is 0.01%, 0.02%, 0.03%, 0.04%, 0.05%, 0.06%, 0.07%, 0.08%, 0.09% or 0.1% polysorbate 85 (w/w). In another embodiment, the final concentration of the polysorbate 85 in the formulation is 1% polysorbate 85 (w/w).

In certain embodiments, the immunogenic composition of the disclosure has a pH of 5.5 to 7.5, more preferably a pH of 5.6 to 7.0, even more preferably a pH of 5.8 to 6.0.

In one embodiment, the present disclosure provides a container filled with any of the immunogenic compositions disclosed herein. In one embodiment, the container is selected from the group consisting of a vial, a syringe, a flask, a fermentor, a bioreactor, a bag, a jar, an ampoule, a cartridge and a disposable pen. In one embodiment, the container is a vial, a syringe, a flask, a fermentor, a bioreactor, a bag, a jar, an ampoule, a cartridge or a disposable pen. In certain embodiments, the container is siliconized.

In an embodiment, the container of the present disclosure is made of glass, metals (e.g., steel, stainless steel, aluminum, etc.) and/or polymers (e.g., thermoplastics, elastomers, thermoplastic-elastomers). In an embodiment, the container of the present disclosure is made of glass.

In one embodiment, the present disclosure provides a syringe filled with any of the immunogenic compositions disclosed herein. In certain embodiments, the syringe is siliconized and/or is made of glass.

A typical dose of the immunogenic composition of the invention for injection has a volume of 0.1 mL to 2 mL, more preferably 0.2 mL to 1 mL, even more preferably a volume of about 0.5 mL.

2.3 Use as Antigens

The polysaccharide purified by the method of the present invention or the conjugates disclosed herein may be use as antigens. For example they may be part of a vaccine. Therefore in an embodiment, the polysaccharides purified by the method of the present invention or the glycoconjugates obtained using said polysaccharides are for use in generating an immune response in a subject. In one aspect, the subject is a mammal, such as a human, cat, sheep, pig, horse, bovine or dog. In one aspect, the subject is a human.

In an embodiment, the polysaccharides purified by the method of the present invention, the glycoconjugates obtained using said polysaccharides or the immunogenic compositions disclosed herein are for use in a vaccine.

In an embodiment, the polysaccharides purified by the method of the present invention, the glycoconjugates obtained using said polysaccharides or the immunogenic compositions disclosed herein are for use as a medicament.

The immunogenic compositions described herein may be used in therapeutic or prophylactic methods for preventing, treating or ameliorating a bacterial infection, disease or condition in a subject. In particular, immunogenic compositions described herein may be used to prevent, treat or ameliorate a S. pneumoniae serotype 3 infection, disease or condition in a subject.

Thus, in one aspect, the disclosure provides a method of preventing, treating or ameliorating an infection, disease or condition associated with S. pneumoniae serotype 3 in a subject, comprising administering to the subject an immunologically effective amount of an immunogenic composition of the disclosure (in particular an immunogenic composition comprising the corresponding polysaccharide or glycoconjugate thereof). In an embodiment, the disclosure provides a method of inducing an immune response to S. pneumoniae serotype 3 in a subject comprising administering to the subject an immunologically effective amount of an immunogenic composition of the disclosure. In an embodiment, the immunogenic compositions disclosed herein are for use as a vaccine. In such embodiments the immunogenic compositions described herein may be used to prevent S. pneumoniae serotype 3 infection in a subject. Thus, in one aspect, the invention provides a method of preventing an infection by S. pneumoniae serotype 3 in a subject comprising administering to the subject an immunologically effective amount of an immunogenic composition of the disclosure.

In one aspect, the subject is a mammal, such as a human, cat, sheep, pig, horse, bovine or dog. In one aspect, the subject is a human.

The immunogenic compositions of the present disclosure can be used to protect or treat a human susceptible to a S. pneumoniae serotype 3 infection, by means of administering the immunogenic compositions via a systemic or mucosal route. In an embodiment, the immunogenic compositions disclosed herein are administered by intramuscular, intraperitoneal, intradermal or subcutaneous routes. In an embodiment, the immunogenic compositions disclosed herein are administered by intramuscular, intraperitoneal, intradermal or subcutaneous injection. In an embodiment, the immunogenic compositions disclosed herein are administered by intramuscular or subcutaneous injection.

In some cases, as little as one dose of the immunogenic composition according to the disclosure is needed, but under some circumstances, such as conditions of greater immune deficiency, a second, third or fourth dose may be given. Following an initial vaccination, subjects can receive one or several booster immunizations adequately spaced.

In an embodiment, the schedule of vaccination of the immunogenic composition according to the disclosure is a single dose.

In an embodiment, the schedule of vaccination of the immunogenic composition according to the disclosure is a multiple dose schedule.

3 The Invention Also Provides the Following Embodiments as Defined in the Following Numbered Paragraphs 1 to 169

1. A method for purifying Streptococcus pneumoniae serotype 3 polysaccharide from a solution comprising said polysaccharide together with contaminants, wherein said method comprises a base treatment step.

2. The method of paragraph 1 wherein the contaminants are cell debris.

3. The method of any one of paragraphs 1-2 wherein the contaminants are proteins and nucleic acids.

4. The method of any one of paragraphs 1-2 wherein the contaminants are proteins, C-polysaccharide and nucleic acids.

5. The method of any one of paragraphs 1-3 wherein the solution is a bacterial culture of Streptococcus pneumoniae serotype 3.

6. The method of any one of paragraphs 1-5 wherein the solution is a liquid bacterial culture Streptococcus pneumoniae serotype 3.

7. The method of any one of paragraphs 1-6 wherein the solution is the supernatant from a centrifuged Streptococcus pneumoniae serotype 3 bacterial culture.

8. The method of any one of paragraphs 1-7 wherein the solution is Streptococcus pneumoniae serotype 3 cells in suspension in their original culture medium.

9. The method of any one of paragraphs 1-7 wherein the solution is a wet cell paste.

10. The method of any one of paragraphs 1-7 or 9 wherein the solution is Streptococcus pneumoniae serotype 3 cells resuspended in an aqueous medium.

11. The method of any one of paragraphs 1-10 wherein the solution is treated with a lytic agent.

12. The method of paragraph 11 wherein the lytic agent is a detergent.

13. The method of paragraph 12 wherein the detergent is selected from the group consisting of deoxycholate sodium (DOC), N-lauryl sarcosine (NLS), chenodeoxycholic acid sodium, and saponins.

14. The method of paragraph 12 wherein the detergent is DOC.

15. The method of paragraph 11 wherein the lytic agent is a non-animal derived lytic agent.

16. The method of paragraph 15 wherein the non-animal derived lytic agent is selected from the group consisting of decanesulfonic acid, tert-octylphenoxy 5 poly(oxyethylene)ethanols, octylphenol ethylene oxide condensates, N-lauryl sarcosine sodium (NLS), lauryl iminodipropionate, sodium dodecyl sulfate, chenodeoxycholate, hyodeoxycholate, glycodeoxycholate, taurodeoxycholate, taurochenodeoxycholate, and cholate.

17. The method of paragraph 15 wherein the non-animal derived lytic agent is NLS.

18. The method of any one of paragraphs 1-10 wherein the solution is enzymatically treated such that the polysaccharide is released.

19. The method of paragraphs 18 wherein the bacterial cells are treated by an enzyme selected from the group consisting of lysostaphin, mutanolysin β-N-acetylglucosaminidase and a combination of mutanolysin and β-N-acetylglucosaminidase.

20. The method of paragraphs 18 wherein the bacterial cells are treated by a type II phosphodiesterase (PDE2).

21. The method of any one of paragraphs 1-10 wherein the bacterial cells are autoclaved such that the polysaccharide is released.

22. The method of any one of paragraphs 1-10 wherein the bacterial cells are chemically treated such that the polysaccharide is released.

23. The method of any one of paragraphs 1-22 wherein the solution is treated by a base to achieve a pH above 8.0.

24. The method of any one of paragraphs 1-22 wherein the solution is treated by a base to achieve a pH above 10.0.

25. The method of any one of paragraphs 1-22 wherein the solution is treated by a base to achieve a pH between 8.0 and 14.0.

26. The method of any one of paragraphs 1-22 wherein the solution is treated by a base to achieve a pH above to achieve a pH between 10.0 and 14.0.

27. The method of any one of paragraphs 1-22 wherein the solution is treated by a base to achieve a pH of about 13.0.

28. The method of any one of paragraphs 1-22 wherein the solution is treated by a base to achieve a pH of about 12.0.

29. The method of any one of paragraphs 1-22 wherein the solution is treated by a base to achieve a pH of about 13.0.

30. The method of any one of paragraphs 23-29 wherein the base is selected from the group consisting of NaOH, KOH, LiOH, NaHCO₃, Na2C03, KzC03, KCN, Et3N, NH3, HzN2H2, NaH, NaOMe, NaOEt and KOtBu.

31. The method of any one of paragraphs 23-29 wherein the base is NaOH.

32. The method of any one of paragraphs 23-31 wherein following the addition of the base, the solution is hold for some time prior to downstream processing.

33. The method of any one of paragraphs 23-32 wherein the base treatment step is performed at a temperature between about 4° C. and about 30° C.

34. The method of any one of paragraphs 23-34 wherein, after base treatment, the suspension is clarified by decantation, sedimentation, filtration or centrifugation.

35. The method of any one of paragraphs 23-34 wherein, after base treatment, the suspension is clarified by centrifugation.

36. The method of paragraph 35 wherein, said centrifugation is continuous centrifugation.

37. The method of paragraph 35 wherein, said centrifugation is bucket centrifugation.

38. The method of any one of paragraphs 35-37 wherein, the suspension is centrifuged between about 5,000 g and about 25,000 g.

39. The method of any one of paragraphs 35-38 wherein the suspension is centrifuged during between about 5 and about 380 minutes.

40. The method of any one of paragraphs 23-34 wherein, after base treatment, the suspension is clarified by decantation.

41. The method of any one of paragraphs 23-34 wherein, after base treatment, the suspension is clarified by sedimentation (settling).

42. The method of any one of paragraphs 34-41 wherein the Streptococcus pneumoniae serotype 3 polysaccharide containing solution is further clarified.

43. The method of any one of paragraphs 34-41 wherein the Streptococcus pneumoniae serotype 3 polysaccharide containing solution is filtrated, thereby producing a further clarified solution.

44. The method of any one of paragraphs 34-41 wherein the Streptococcus pneumoniae serotype 3 polysaccharide containing solution is treated by a depth filtration step.

45. The method of any one of paragraphs 34-41 wherein the Streptococcus pneumoniae serotype 3 polysaccharide containing solution is treated by a depth filtration step wherein the depth filter has a nominal retention range of between about 0.01-100 micron.

46. The method of any one of paragraphs 34-45 wherein the Streptococcus pneumoniae serotype 3 polysaccharide containing solution is treated by a depth filtration step wherein the depth filter has a filter capacity of 1-2500 L/m2.

47. The method of any one of paragraphs 34-46 wherein the Streptococcus pneumoniae serotype 3 polysaccharide containing solution is treated by a depth filtration step wherein the feed rate is between 1-1000 LMH (liters/m2/hour).

48. The method of any one of paragraphs 34-47 wherein the Streptococcus pneumoniae serotype 3 polysaccharide containing solution is further clarified by Ultrafiltration and/or Diafiltration.

49. The method of any one of paragraphs 34-47 wherein the Streptococcus pneumoniae serotype 3 polysaccharide containing solution is further clarified by Ultrafiltration and Diafiltration.

50. The method of any one of paragraphs 48-49 wherein the molecular weight cut off of the membrane of Ultrafiltration is in the range of between about 5 kDa-1000 kDa.

51. The method of any one of paragraphs 48-50 wherein the molecular weight cut off of the membrane of Ultrafiltration is in the range of between about 10 kDa-50 kDa.

52. The method of any one of paragraphs 48-51 wherein the molecular weight cut off of the membrane of Ultrafiltration is about 30 kDa.

53. The method of any one of paragraphs 48-52 wherein the concentration factor of the ultrafiltration step is from about 1.5 to 10.

54. The method of any one of paragraphs 48-53 wherein the replacement solution of the Diafiltration step is water.

55. The method of any one of paragraphs 48-53 wherein the replacement solution of the Diafiltration step is saline in water.

56. The method of paragraph 55 wherein the salt is sodium chloride.

57. The method of paragraph 55 wherein the replacement solution is sodium chloride at about 40 mM, about 45 mM, about 50 mM, about 55 mM, about 60 mM, about 65 mM, about 70 mM, about 80 mM, about 90 mM or about 100 mM.

58. The method of any one of paragraphs 48-57 wherein the number of diavolumes is between 5 and 20.

59. The method of any one of paragraphs 23-58 wherein the Streptococcus pneumoniae serotype 3 polysaccharide containing solution is further treated by a flocculation step.

60. The method of paragraph 59 wherein said flocculation step comprises adding a flocculating agent to the solution.

61. The method of any one of paragraphs 59-60 wherein said flocculation step include adjustment of the pH.

62. The method of paragraph 61 wherein adjustment of the pH is conducted before the addition of the flocculating agent.

63. The method of paragraph 61 wherein adjustment of the pH is conducted after the addition of the flocculating agent.

64. The method of any one of paragraphs 60-63 wherein the addition of the flocculating agent and/or the adjustment of the pH is performed at a temperature adjusted to a desirable level.

65. The method of any one of paragraphs 60-63 wherein the addition of the flocculating agent and the adjustment of the pH is performed at a temperature adjusted to a desirable level.

66. The method of paragraph 59 wherein said flocculation step comprises adding a flocculating agent to the solution, adjustment of the pH and adjustment of the temperature.

67. The method of paragraph 66 wherein addition of the flocculating agent is followed by adjustment of the pH and followed by adjustment of the temperature.

68. The method of paragraph 66 wherein addition of the flocculating agent is followed by adjustment of the temperature and followed by adjustment of the pH.

69. The method of paragraph 66 wherein adjustment of the pH is followed by addition of the flocculating agent and followed by adjustment of the temperature.

70. The method of paragraph 66 wherein adjustment of the pH is followed by adjustment of the temperature and followed by addition of the flocculating agent.

71. The method of paragraph 66 wherein adjustment of the temperature is followed by addition of the flocculating agent and followed by adjustment of the pH.

72. The method of paragraph 66 wherein adjustment of the temperature is followed by adjustment of the pH and followed by addition of the flocculating agent.

73. The method of any one of paragraphs 60-72 wherein following the addition of the flocculating agent and/or the adjustment of the pH, the solution is hold for some time to allow settling of the flocs.

74. The method of any one of paragraphs 60-72 wherein said flocculating agent comprises a multivalent cation.

75. The method of any one of paragraphs 60-72 wherein said flocculating agent is a multivalent cation.

76. The method of any one of paragraphs 60-72 wherein said flocculating agent is a multivalent cation selected from the group consisting of aluminium, iron, calcium and magnesium.

77. The method of any one of paragraphs 60-72 wherein said flocculating agent is a mixture of at least two multivalent cations selected from the group consisting of aluminium, iron, calcium and magnesium.

78. The method of any one of paragraphs 60-72 wherein said flocculating agent comprises an agent selected from the group consisting of magnesium chloride, alum, aluminium chlorohydrate, aluminium sulphate, calcium oxide, calcium hydroxide, iron(II) sulphate (ferrous sulphate), iron(III) chloride (ferric chloride), polyacrylamide, modified polyacrylamides, polyDADMAC, polyethylenimine (PEI), sodium aluminate and sodium silicate.

79. The method of any one of paragraphs 60-72 wherein said flocculating agent is selected from the group consisting of alum, aluminium chlorohydrate, aluminium sulphate, magnesium chloride, calcium oxide, calcium hydroxide, iron(II) sulphate (ferrous sulphate), iron(III) chloride (ferric chloride), polyacrylamide, modified polyacrylamides, polyDADMAC, sodium aluminate and sodium silicate.

80. The method of any one of paragraphs 60-72 wherein said flocculating agent is aluminium sulphate.

81. The method of any one of paragraphs 60-72 wherein said flocculating agent is sodium aluminate.

82. The method of any one of paragraphs 60-72 wherein said flocculating agent is aluminium chlorohydrate.

83. The method of any one of paragraphs 60-72 wherein said flocculating agent comprises alum.

84. The method of any one of paragraphs 60-72 wherein said flocculating agent is alum.

85. The method of any one of paragraphs 60-72 wherein said flocculating agent is potassium alum.

86. The method of any one of paragraphs 60-72 wherein said flocculating agent is sodium alum.

87. The method of any one of paragraphs 60-72 wherein said flocculating agent is ammonium alum.

88. The method of any one of paragraphs 60-87 wherein a concentration of flocculating agent of between about 10 and 200 mM is used.

89. The method of any one of paragraphs 59-88 wherein said flocculation step is performed at a pH below 7.0.

90. The method of any one of paragraphs 59-88 wherein said flocculation step is performed at a pH between 7.0 and 1.0.

91. The method of any one of paragraphs 59-88 wherein said flocculation step is performed at a pH between 5.5 and 3.5.

92. The method of any one of paragraphs 59-91 wherein said flocculation step is performed at acidic pH and wherein said acidic pH is obtained by acidifying the solution with an acid.

93. The method of paragraph 92 wherein said acid is selected from the group consisting of HCl, H3PO4, citric acid, acetic acid, nitrous acid, and sulfuric acid.

94. The method of paragraph 92 wherein said acid is hydrochloric acid.

95. The method of paragraph 92 wherein said acid is sulfuric acid.

96. The method of any one of paragraphs 59-95 wherein following the addition of the flocculating agent and the acidification if present, the solution is hold for some time to allow settling of the flocs.

97. The method of any one of paragraphs 59-96 wherein the addition of the flocculating agent, the settling of the solution and/or the adjustment of the pH is performed at a temperature between about 4° C. and about 30° C.

98. The method of any one of paragraphs 59-96 wherein the addition of the flocculating agent, the settling of the solution and/or the adjustment of the pH is performed at a temperature between about 45° C. to about 65° C.

99. The method of any one of paragraphs 59-96 wherein the addition of the flocculating agent is performed at a temperature between about 4° C. and about 30° C.

100. The method of any one of paragraphs 59-96 wherein the addition of the flocculating agent is performed at a temperature between about 45° C. to about 65° C.

101. The method of any one of paragraphs 59-96 wherein the settling of the solution after the addition of the flocculating agent is performed at a temperature between about 4° C. and about 30° C.

102. The method of any one of paragraphs 59-96 wherein the settling of the solution after the addition of the flocculating agent is performed at a temperature between about 45° C. to about 65° C.

103. The method of any one of paragraphs 59-96 wherein the adjustment of the pH is performed at a temperature between about 4° C. and about 30° C.

104. The method of any one of paragraphs 59-96 wherein the adjustment of the pH is performed at a temperature between about 45° C. to about 65° C.

105. The method of anyone of paragraphs 59-96 wherein the addition of the flocculating agent, the settling of the solution and the adjustment of the pH are performed at a temperature between about 4° C. and about 30° C.

106. The method of anyone of paragraphs 59-96 wherein the addition of the flocculating agent, the settling of the solution and the adjustment of the pH are performed at a temperature between about 45° C. to about 65° C.

107. The method of any one of paragraphs 59-106 wherein after flocculation, the suspension is clarified by decantation, sedimentation, filtration or centrifugation.

108. The method of any one of paragraphs 59-106 wherein after flocculation, the suspension is clarified by centrifugation.

109. The method of paragraph 108 wherein said centrifugation is continuous centrifugation.

110. The method of paragraph 108 wherein said centrifugation is bucket centrifugation.

111. The method of any one of paragraphs 108-110 wherein the suspension is centrifuged between about 5,000 g and about 25,000 g.

112. The method of any one of paragraphs 108-111 wherein the suspension is centrifuged during between about 5 and about 600 minutes

113. The method of any one of paragraphs 59-106 wherein after flocculation, the suspension is clarified by filtration.

114. The method of any one of paragraphs 59-106 wherein after flocculation, the suspension is clarified by sedimentation.

115. The method of any one of paragraphs 59-106 wherein after flocculation, the suspension is clarified by decantation.

116. The method of any one of paragraphs 59-115 wherein after the flocculation step and/or the solid/liquid separation step, the pH of the polysaccharide containing solution is adjusted to a pH above 5.0.

117. The method of any one of paragraphs 59-115 wherein after the flocculation step and/or the solid/liquid separation step, the pH of the polysaccharide containing solution is adjusted to a pH between 5.0 and 9.0.

118. The method of any one of paragraphs 59-115 wherein after the flocculation step and/or the solid/liquid separation step, the pH of the polysaccharide containing solution is adjusted to a pH between 6.0 and 8.0.

119. The method of any one of paragraphs 59-115 wherein after the flocculation step and/or the solid/liquid separation step, the pH of the polysaccharide containing solution is adjusted to a pH between 6.5 and 7.5.

120. The method of any one of paragraphs 59-115 wherein after the flocculation step and/or the solid/liquid separation step, the pH of the polysaccharide containing solution is adjusted to a pH of about 7.0.

121. The method of any one of paragraphs 116-120 wherein said pH is raised by the addition of a base selected from the group consisting of NaOH, KOH, LiOH, NaHCO₃, Na2C03, KzC03, KCN, Et3N, NH3, HzN2H2, NaH, NaOMe, NaOEt and KOtBu.

122. The method of any one of paragraphs 116-120 wherein said pH is raised by the addition of KOH.

123. The method of any one of paragraphs 116-120 wherein said pH is raised by the addition of NaOH.

124. The method of any one of paragraphs 59-123 wherein the solution containing the polysaccharide is further clarified by an activated carbon filtration step.

125. The method of paragraph 124 wherein the solution is filtered through activated carbon immobilized in a matrix.

126. The method of paragraph 125 wherein the activated carbon immobilized in the matrix is in the form of a flow-through carbon cartridge.

127. The method of paragraph 125 or 126 wherein the activated carbon immobilized in a matrix is placed in a housing to form an independent filter unit.

128. The method of paragraph 127 wherein said filter unit is filter unit a CUNO zetacarbon filter.

129. The method of any one of paragraphs 127-128 wherein the activated carbon filter has a nominal micron rating of between about 0.01-100 micron.

130. The method of any one of paragraphs 124-129 wherein the activated carbon filtration step is conducted at a feed rate of between 1-500 LMH.

131. The method of any one of paragraphs 124-130 wherein the solution is treated by an activated carbon filter wherein the filter has a filter capacity of between 5-1000 L/m2.

132. The method of any one of paragraphs 124-131 wherein said activated carbon filtration step is repeated.

133. The method of any one of paragraphs 124-131 wherein 1, 2, 3, 4, 5, 6, 7, 8, 9, or activated carbon filtration step(s) are performed.

134. The method of any one of paragraphs 124-131 wherein the solution is treated by activated carbon filters in series.

135. The method of any one of paragraphs 124-131 wherein the solution is treated by 1, 2, 3, 4, 5, 6, 7, 8, 9, or 10 activated carbon filters in series.

136. The method of any one of paragraphs 124-135 wherein the activated carbon filtration step is performed in a single pass mode.

137. The method of any one of paragraphs 124-135 wherein the activated carbon filtration step is performed in recirculation mode.

138. The method of any one of paragraphs 107-137 wherein the Streptococcus pneumoniae serotype 3 polysaccharide containing solution is further clarified by Ultrafiltration and/or Diafiltration.

139. The method of any one of paragraphs 107-137 wherein the Streptococcus pneumoniae serotype 3 polysaccharide containing solution is further clarified by Ultrafiltration and Diafiltration.

140. The method of any one of paragraphs 138-139 wherein the molecular weight cut off of the membrane of Ultrafiltration is in the range of between about 5 kDa-1000 kDa.

141. The method of any one of paragraphs 138-139 wherein the molecular weight cut off of the membrane of Ultrafiltration is in the range of between about 10 kDa-50 kDa.

142. The method of any one of paragraphs 138-139 wherein the molecular weight cut off of the membrane of Ultrafiltration is about 30 kDa.

143. The method of any one of paragraphs 138-139 wherein the concentration factor of the ultrafiltration step is from about 1.5 to 10.

144. The method of any one of paragraphs 138-143 wherein the replacement solution of the Diafiltration step is water.

145. The method of any one of paragraphs 138-143 wherein the replacement solution of the Diafiltration step is saline in water.

146. The method of paragraph 145 wherein the salt is sodium chloride.

147. The method of paragraph 146 wherein the replacement solution is sodium chloride at about 40 mM, about 45 mM, about 50 mM, about 55 mM, about 60 mM, about 65 mM, about 70 mM, about 80 mM, about 90 mM or about 100 mM.

148. The method of any one of paragraphs 138-143 wherein the replacement solution of the Diafiltration step is a buffer solution.

149. The method of paragraph 148 wherein the diafiltration buffer is selected from the group consisting of a salt of acetic acid (acetate), a salt of citric acid (citrate), a salt of formic acid (formate), a salt of malic acid (Malate), a salt of maleic acid (Maleate), a salt of phosphoric acid (Phosphate) and a salt of succinic acid (Succinate).

150. The method of any one of paragraphs 148-149 wherein the pH of the diafiltration buffer is between about 6.0-7.5.

151. The method of any one of paragraphs 148-149 wherein the concentration of the diafiltration buffer is between about 0.01 mM-100 mM.

152. The method of any one of paragraphs 144-151 wherein the replacement solution comprises a chelating agent.

153. The method of paragraph 152 wherein said chelating agent is Ethylene Diamine Tetra Acetate (EDTA).

154. The method of any one of paragraphs 152-153 wherein the chelating agent is employed at a concentration from 1 to 500 mM.

155. The method of any one of paragraphs 148-154 wherein the diafiltration buffer solution comprises a salt.

156. The method of paragraph 155 wherein the salt is selected from the group consisting of magnesium chloride, potassium chloride, sodium chloride and a combination thereof.

157. The method of any one of paragraphs 138-156 wherein the number of diavolumes is between 5 and 20.

158. The method of any one of paragraphs 138-157 wherein the Ultrafiltration and/or Diafiltration steps are performed at a temperature between about 20° C. to about 90° C.

159. The method of any one of paragraphs 1-158 wherein the purified solution of polysaccharide is sized to a target molecular weight.

160. The method of any one of paragraphs 1-159 wherein the purified solution of polysaccharide is sized by mechanical sizing.

161. The method of any one of paragraphs 1-159 wherein the purified solution of polysaccharide is sized by chemical hydrolysis.

162. The method of any one of paragraphs 1-161 wherein the purified solution of polysaccharide is sterilely filtered.

163. The method of paragraph 1-161 wherein the purified solution of polysaccharide is treated by a sterile filtration step wherein the filter has a nominal retention range of between about 0.01-0.2 micron.

164. The method of paragraph 1-161 wherein the purified solution of polysaccharide is treated by a sterile filtration step wherein the filter has a nominal retention range of between about 0.15-0.2 micron.

165. The method of paragraph 1-161 wherein the purified solution of polysaccharide is treated by a sterile filtration step wherein the filter has a nominal retention range of about 0.2 micron.

166. The method of paragraph 1-165 wherein the purified solution is treated by a sterile filtration step wherein the filter has a filter capacity of about 25-1500 L/m2.

167. The method of paragraph 1-166 wherein the purified S. pneumoniae serotype 3 polysaccharide is prepared as a liquid solution.

168. The method of paragraph 1-166 wherein the purified S. pneumoniae serotype 3 polysaccharide is further processed.

169. The method of paragraph 168 wherein the purified S. pneumoniae serotype 3 polysaccharide is lyophilized as a dried powder

As used herein, the term “about” means within a statistically meaningful range of a value, such as a stated concentration range, time frame, molecular weight, temperature or pH. Such a range can be within an order of magnitude, typically within 20%, more typically within 10%, and even more typically within 5% or within 1% of a given value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art.

Whenever a range is recited within this application, every number within the range is also contemplated as an embodiment of the disclosure.

The terms “comprising”, “comprise” and “comprises” herein are intended by the inventors to be optionally substitutable with the terms “consisting essentially of”, “consist essentially of”, “consists essentially of”, “consisting of”, “consist of” and “consists of”, respectively, in every instance.

An “immunogenic amount”, an “immunologically effective amount”, a “therapeutically effective amount”, a “prophylactically effective amount”, or “dose”, each of which is used interchangeably herein, generally refers to the amount of antigen or immunogenic composition sufficient to elicit an immune response, either a cellular (T cell) or humoral (B cell or antibody) response, or both, as measured by standard assays known to one skilled in the art.

Any whole number integer within any of the ranges of the present document is contemplated as an embodiment of the disclosure.

All references or patent applications cited within this patent specification are incorporated by reference herein.

The invention is illustrated in the accompanying examples. The examples below are carried out using standard techniques, which are well known and routine to those of skill in the art, except where otherwise described in detail. The examples are illustrative, but do not limit the invention.

### EXAMPLE

### Example 1. Purification of Pneumococcal Polysaccharide Serotype 3

The process flow diagram for the purification is shown in FIG. 1. The process begins with NLS inactivated fermentation broth (see e.g. EP2129693).

1. Starting Material

The process begins with N-Lauroylsarcosine sodium (NLS) inactivated fermentation broth of S. pneumonia serotype 3. Cultures were grown in Hy-Soy medium. At the end of growth (as indicated by no further increase in optical density), the cultures were inactivated with NLS (see e.g. EP2129693).

2. Base Treatment

The NLS lysed broth was treated with 10N NaOH solution to a final NaOH concentration of 0.5M. The main purpose of this step is to render the negatively charged polysaccharide to be more soluble in the presence of other impurities such as protein and nucleic acids. The NaOH treated broth was incubated at ambient temperature overnight.

3. Centrifugation

The base treated broth from Step 2 above was subjected to centrifugation which removed the insoluble cell debris and other particles. The centrifugation was conducted at 10,000-xg for 30-60 mins at 20° C.

The supernatant was collected.

4. Optional Depth Filtration

Although centrifugation is the primary solid/liquid separation unit operation, it does not remove all of the particles, a depth filtration unit operation was incorporated between the centrifugation and the first ultrafiltration unit operation.

5. Ultrafiltration/Diafiltration-(UFDF-1)

Purification begins with the depth filtrate (from step 4 above).

The clarified cell lysate is concentrated and diafiltered (UFDFed).

The alkaline centrate from Step 3 or 4 above is subjected to ultrafiltration and diafiltration using 30 kDa MWCO membrane. The broth is concentrated by about 8-9 folds and then is diafiltered with triplet buffer systems: first with 0.6M NaCl in 0.1N NaOH pH 12.5 followed by 50 mM NaCl in 0.1N NaOH pH 12 to keep the polysaccharide in a soluble (non-complexed) state and allow further removal of impurities. Finally, the batch is diafiltered into the water. This step is performed using a 30-kDa molecular weight cutoff filter.

After diafiltration, the retentate was recovered by draining from the filter apparatus.

6. Acidification/Flocculation

After buffer exchange, 1M MgCl₂ is added to the serotype 3 polysaccharide containing solution to a final concentration of 20 mM. The pH of the solution is adjusted to about 3.9 with 5N H₂SO₄.

A white precipitate was formed. The flocculated solution is held at room temperature for at least 1 hr before clarification.

7. Centrifugation

The white precipitate was removed by centrifugation conducted at 10,000-xg for 30 minutes at 20° C. The supernatant was collected.

Although optional, a 0.2 micron filter was used in some samples to remove remaining turbidity (if present).

8. Neutralization

The pH of the solution was slowly adjusted to a pH of about 7.0 with 0.5 N or 0.1 N NaOH and filtered with a 0.2 micron filter.

9. Carbon Filtration

This unit operation reduces the level of host cell impurities such as proteins and nucleic acids as well as colored impurities. The neutralized centrate from step 8 is filtered through one 7″ diameter R32SP carbon filter at a flow rate of 40 LMH. The filtrate that contained the product was collected.

10. Ultrafiltration/Diafiltration-(UFDF-2)

This unit operation concentrates the product to the desired concentration and replace the buffer that contained MgCl2 and other salts with water for subsequent use. This step is performed using a 30-kDa molecular weight cutoff filter. The retentate along with the rinse were combined and 0.2-μm filtered. The final 0.2-μm bulk filtrate was then put in the storage bottles.

### Example 2. Analysis of Purified Pneumococcal Polysaccharide Serotype 3

The process of Example 1 has been found to result in a much shorter purification process as compared to known processes. The new process has only 8-unit operations.

The protein/polysaccharide ratio was found to be as low as about 0.3%. The process of the invention was found to generate polysaccharide with similar nucleic acid and C-poly levels compared with the process of the prior art.

The analytical results showing the residual impurities are shown in Table 1. All three batches met all of the pre-defined acceptance criteria.

TABLE 1
Analytical Results and Overall Yields for Three Batches
using the purification process of Example 1

| | Batches and size (in L) | | |
|---|---|---|---|
| | 1 (10 L) | 2 (20 L) | 3 (2 L) |
| Mwt (kDa) | 170 | 320 | 565 |
| Residual Protein (%) | 0.7 | 0.3 | 0.29 |
| % Nucleic Acid | 0.05 | 0.04 | 0.06 |
| % Yield | 60 | 65 | 45 |
| % Residual C poly | Low by NMR | Low by NMR | 0.86 |

The SEC-HPLC peak profiles after each unit operation are shown in FIG. 2. The RI trace shows the polysaccharide peak and the UV 280 trace shows protein peaks. The final (UF2) sample has very low remaining residual protein left, which indicates efficient protein removal. Also, the RI profile shows a single symmetrical poly peak which is an indication of purity (see FIG. 2).

All publications and patent applications mentioned in the specification are indicative of the level of those skilled in the art to which this invention pertains. All publications and patent applications are hereby incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

1. A method for purifying Streptococcus pneumoniae serotype 3 polysaccharide from a solution comprising said polysaccharide together with contaminants, wherein said method comprises a base treatment step.

2. The method of claim 1 wherein the solution is treated by a base to achieve a pH above 8.0.

3. The method of claim 2 wherein the base comprises at least one of NaOH, KOH, LiOH, NaHC03, Na2C03, KzC03, KCN, Et3N, NH3, HzN2H2, NaH, NaOMe, NaOEt or KOtBu.

4. The method of claim 2 wherein, after base treatment, the suspension is clarified by decantation, sedimentation, filtration or centrifugation.

5. The method of claim 4 wherein the Streptococcus pneumoniae serotype 3 polysaccharide containing solution is filtrated, thereby producing a further clarified solution.

6. The method of claim 4 wherein the Streptococcus pneumoniae serotype 3 polysaccharide containing solution is treated by a depth filtration step.

7. The method of claim 4 wherein the Streptococcus pneumoniae serotype 3 polysaccharide containing solution is further clarified by Ultrafiltration and/or Diafiltration.

8. The method of claim 2 wherein the Streptococcus pneumoniae serotype 3 polysaccharide containing solution is further treated by a flocculation step comprising adding a flocculating agent to the solution.

9. The method of claim 8 wherein said flocculating agent is a multivalent cation.

10. The method of claim 9 wherein said multivalent cation comprises at least one of aluminium, iron, calcium or magnesium.

11. The method of claim 8 wherein said flocculation step is performed at a pH below 7.0.

12. The method of claim 8 wherein after said flocculation step, the suspension is clarified by decantation, sedimentation, filtration or centrifugation.

13. The method of claim 8 wherein after said flocculation step, the pH of the polysaccharide containing solution is adjusted to a pH above 5.0.

14. The method of claim 8 wherein the polysaccharide containing solution is further clarified by an activated carbon filtration step.

15. The method of claim 12 wherein the Streptococcus pneumoniae serotype 3 polysaccharide containing solution is further clarified by Ultrafiltration and/or Diafiltration.

16. The method of claim 1 wherein the purified solution of polysaccharide is sized to a target molecular weight.

17. The method of claim 1 wherein the purified solution of polysaccharide is sterilely filtered.

18. The method of claim 12 wherein after said decantation, sedimentation, filtration or centrifugation step, the pH of the polysaccharide containing solution is adjusted to a pH above 5.0.
